(19) United States
(12) Patent Application Publication
(10) Pub. No.: US 2013/0274215 A1
(43) Pub. Date: Oct. 17, 2013

(54) PHARMACEUTICAL COMPOSITIONS TO TREAT FIBROSIS

(71) Applicants: R. Scott Thies, San Diego, CA (US); Francine S. Farouz, La Jolla, CA (US); David Jenkins, San Diego, CA (US)

(72) Inventors: R. Scott Thies, San Diego, CA (US); Francine S. Farouz, La Jolla, CA (US); David Jenkins, San Diego, CA (US)

(73) Assignee: FATE THERAPEUTICS, INC., San Diego, CA (US)

(21) Appl. No.: 13/639,644

(22) PCT Filed: Apr. 6, 2011
(86) PCT No.: PCT/US11/31411

§ 371 Date: May 28, 2013

Related U.S. Application Data
(60) Provisional application No. 61/322,233, filed on Apr. 8, 2010.

(51) Int. Cl. A61K 31/47; A61K 31/428; A61K 31/277; A61K 31/4709; A61K 31/551; A61K 31/502; A61K 31/706; A61K 31/194; A61K 31/4375; A61K 31/337; A61K 31/165; A61K 31/444; A61K 31/429; A61K 31/58; A61K 31/7048; A61K 31/4985

(52) U.S. Cl. CPC A61K 31/47; A61K 31/4985; A61K 31/428; A61K 31/277; A61K 31/4709; A61K 31/551; A61K 31/502; A61K 31/706; A61K 31/194; A61K 31/4375; A61K 31/337; A61K 31/165; A61K 31/444; A61K 31/429; A61K 31/58; A61K 31/7048
USPC 514/30; 514/250; 514/367; 514/523; 514/314; 514/218; 514/248; 514/43; 514/568; 514/283; 514/449; 514/629; 514/311; 514/333; 514/368; 514/172; 435/375

(57) ABSTRACT

The present invention provides methods for the prevention, treatment and/or amelioration of fibrosis or fibrotic conditions. The present invention further provides small molecule inhibitors of Wnt- and TGF-p-mediated β-catenin signaling to prevent, treat and/or ameliorate fibrosis or fibrotic conditions. Kits comprising small molecule inhibitors of Wnt- and TGF-p-mediated β-catenin signaling and methods of identifying small molecule inhibitors of Wnt- and TGF-p-mediated β-catenin signaling are also provided.

### CROSS REFERENCE TO RELATED APPLICATIONS

This application claims benefit under 35 U.S.C. §119(e) of U.S. Provisional Application No. 61/322,233, filed Apr. 8, 2010, which is incorporated by reference in its entirety.

### BACKGROUND

1. Technical Field

The present invention generally relates to methods for the treatment and/or amelioration of fibrosis or fibrotic conditions. More specifically, the invention relates to the use of inhibitors of both Wnt- and TGF-β-mediated β-catenin signaling to treat and/or ameliorate fibrosis or fibrotic conditions.

2. Description of the Related Art

Fibrosis includes pathological conditions characterized by abnormal and/or excessive accumulation of fibrotic material (e.g., extracellular matrix) following tissue damage. Fibroproliferative disease is responsible for morbidity and mortality associated with vascular diseases, such as cardiac disease, cerebral disease, and peripheral vascular disease, and with organ failure in a variety of chronic diseases affecting the pulmonary system, renal system, eyes, cardiac system, hepatic system, digestive system, and skin (Wynn, Nature Reviews. 2004; 4:583-594). However, to date, there are no therapies on the market that are effective in treating or preventing fibrotic disease.

Accordingly, the ineffective treatment of various fibroproliferative diseases, including those affecting the kidney, liver, and lung, has resulted in an enormous burden on the U.S. health care system. For example, an estimated 13% of Americans (29 million) have chronic kidney disease (CKD). CKD is progressive, not curable, and ultimately fatal. Fibrosis is the final pathway in CKD that leads to disease progression and ultimately organ failure. In 2005, CKD including end-stage renal disease (ESRD) accounted for 27% of Medicare expenses ($60 billion) and 36% of care for patients dually covered by Medicare and Medicaid ($18 billion).

Liver fibrosis is a scarring process initiated in response to chronic liver disease (CLD) caused by continuous and repeated insults to the liver. Later stages of CLD are characterized by extensive remodeling of the liver architecture and chronic organ failure, regardless of the underlying disease (e.g., cirrhosis, nonalcoholic steatohepatitis (NASH), primary sclerosing cholangitis (PSC)). For example, recent studies suggest that NASH results in fibrosis in up to 40% of patients and cirrhosis in 5-10% and has a progression rate of 20% over a decade. In light of the growing obesity epidemic worldwide, approximately 12.2 million NASH patients that do not currently receive treatment for liver fibrosis (estimated population that will develop cirrhosis over the next decade) could benefit from anti-fibrotic therapy.

Idiopathic pulmonary fibrosis (IPF) is the main form of lung fibrosis. IPF is a debilitating and life-threatening lung disease characterized by a progressive scarring of the lungs that hinders oxygen uptake. There are an estimated 100,000 plus cases of IPF in the U.S. No FDA-approved treatments for IPF exist, and approximately two-thirds of patients die within five years after diagnosis. Patients with IPF are typically treated with anti-inflammatory agents; however, none have been clinically proven to improve survival or quality of life for patients with IPF.

Systemic sclerosis is a degenerative disorder in which excessive fibrosis occurs in multiple organ systems, including the skin, blood vessels, heart, lungs, and kidneys. Several forms of fibrotic diseases cause death in scleroderma patients, including pulmonary fibrosis, congestive heart failure, and renal fibrosis; each of which occurs in about half of systemic sclerosis patients. The annual incidence of systemic sclerosis is estimated to be 19 cases per million population. Currently, no effective therapies for this life-threatening disease exist.

Fibrosis is also a leading cause of organ transplant rejection. The precise manifestations of chronic rejection vary according to the transplanted organ, but all exhibit proliferation of myofibroblasts, or related cells, ultimately resulting in fibrosis that leads to loss of function. In 2005, over 50,000 solid organ transplants were conducted in the US, Japan and five major European markets. The total number of transplant procedures is expected to increase to more than 67,000 by 2015. The number of patients living with functional grafts in the US alone at year-end 2005 was nearly 164,000. While remarkable progress has been made in the ability to transplant various organs, long term preservation (greater than one year) of organ function and patient survival suffers primarily because of chronic rejection. At this time, no drugs are available for treatment of the fibrotic lesions of progressive chronic allograft rejection.

Existing methods for treating fibroproliferative diseases target the inflammation response which is believed to play a role in the development of fibrosis generally (Wynn, Nature Reviews. 2004; 4:583-594). Examples of pharmaceutical strategies for treating fibrosis include the use of immunosuppressive drugs, such as corticosteroids, other traditional immunosuppressive or cytotoxic agents and antifibrotics.

Nevertheless, despite its enormous impact on human health, there are currently no approved treatments that directly target the mechanism(s) of fibrosis. Thus, a need exists in the art for new and more specifically targeted approaches for the treatment of fibrosis conditions.

### BRIEF SUMMARY

The present invention contemplates, in part, to provide compositions comprising inhibitors of cell signaling pathways that underlie the mechanism(s) of fibrosis that are common to most tissues, including without limitation, cell signaling pathways associated with EMT/EnMT, myofibroblast activation, and myofibroblast deposition of extracellular matrix.

In one embodiment, the present invention provides, in part, a method of preventing or reducing fibrosis comprising inhibiting Wnt- and TGF-β-mediated β-catenin signaling.

In a particular embodiment, a method of preventing or reducing fibrosis comprises administering one or more inhibitors of β-catenin signaling, wherein the inhibitor inhibits Wnt- and TGF-β-mediated β-catenin signaling.

In a certain embodiment, the fibrosis is associated with a fibroproliferative disease selected from the group consisting of: kidney fibrosis, liver fibrosis, lung fibrosis, and systemic sclerosis.

In a certain particular embodiment, the fibroproliferative disease is idiopathic pulmonary fibrosis.

In a particular embodiment, the present invention provides, in part, a method of preventing or treating lung fibrosis in a subject comprising administering one or more inhibitors of β-catenin signaling to the subject, wherein the inhibitor inhibits Wnt- and TGFβ-mediated β-catenin signaling.

In related embodiments, the interstitial lung fibrosis is idiopathic pulmonary fibrosis.

In a particular embodiment, the present invention provides, in part, a method of inhibiting epithelial to mesenchymal transition (EMT) in an epithelial cell comprising contacting the epithelial cell with an inhibitor of β-catenin signaling, wherein the inhibitor inhibits Wnt- and TGF-β-mediated β-catenin signaling.

In certain embodiments, the cell is a lung cell, a kidney cell, or a liver cell.

In another particular embodiment, the present invention provides, in part, a method of inhibiting endothelial to mesenchymal transition (EnMT) in an endothelial cell comprising contacting the endothelial cell with an inhibitor of β-catenin signaling, wherein the inhibitor inhibits Wnt- and TGF-β-mediated β-catenin signaling.

In certain embodiments, the cell is a lung cell, a kidney cell, or a liver cell.

In one embodiment, the present invention provides, in part, a method of inhibiting myofibroblast activation in a myofibroblast comprising contacting the myofibroblast with an inhibitor of β-catenin signaling, wherein the inhibitor inhibits Wnt- and TGF-β-mediated β-catenin signaling.

In certain embodiments, the myofibroblast is a present in a lung tissue, a kidney tissue, or a liver tissue.

In various embodiments, the present invention contemplates, in part, a pharmaceutical composition comprising an inhibitor of β-catenin signaling, wherein the inhibitor inhibits Wnt- and TGF-β-mediated β-catenin signaling, and a pharmaceutically acceptable carrier or excipient, wherein the composition prevents or reduces fibrosis.

In particular embodiments, the fibrosis is associated with a fibroproliferative disease selected from the group consisting of: kidney fibrosis, liver fibrosis, lung fibrosis, and systemic sclerosis.

In other particular embodiments, the fibroproliferative disease is idiopathic pulmonary fibrosis

In one particular embodiment, the present invention provides, in part, a method for identifying an inhibitor of Wnt- and TGF-β-mediated β-catenin signaling, comprising: activating Wnt-mediated β-catenin signaling in a cell and measuring the level of Wnt-mediated β-catenin signaling in the presence and absence of a test compound; activating TGF-β-mediated β-catenin signaling in the cell and measuring the level of TGFβ-mediated β-catenin signaling in the presence and absence of the test compound; comparing the levels of β-catenin signaling measured in step a) and step b) in the presence and absence of the test compound; and identifying the test compound as a Wnt- and TGF-β-mediated β-catenin signaling by observing decreases in the levels of Wnt-mediated β-catenin signaling and TGF-β-mediated β-catenin signaling in the cell, in the presence of the test compound compared to the respective levels in the absence of the test compound.

In various embodiments, methods and compositions of the present invention comprise an inhibitor that comprises a small molecule.

In other various embodiment, methods and compositions of the present invention comprise an inhibitor selected from the group consisting of: FT-1055-3, FT-1067-3, FT-1069-1, FT-1083-1, FT-1147-3, FT-1150-3, FT-1202-1, FT-1203-1, FT-1812-4, FT-1265-1, FT-1281-1, FT-1294-5, FT-1301-1, FT-1320-1, FT-1355-2, FT-1361-2, FT-1366-2, FT-1398-2, FT-1434-2, FT-1435-2, FT-1436-1, FT-1480-1, FT-1497-1, FT-1504-3, FT-1515-1, FT-1517-1, FT-1518-1, FT-1532-1, FT-1575-2, FT-1609-1, FT-1612-3, FT-1613-1, FT-1660-1, FT-1678-1, FT-1688-1, FT-1693-1, FT-1812-3, FT-1915-2, FT-1986-3, FT-1992-3, FT-2014-2, FT-2046-2, FT-2051-2, FT-2081-2, FT-2103-2, FT-2115-2, FT-2228-3, FT-2254-2, FT-2318-2, FT-2342-2, FT-2474-2, FT-2498-2, FT-2562-3, FT-2580-2, FT-2619-2, FT-2633-2, FT-2660-2, FT-2691-2, FT-2693-3, FT-2770-2, FT-2820-2, FT-2862-2, FT-2863-2, FT-2907-2, FT-2909-2, FT-2912-3, FT-2920-3, FT-2947-2, FT-2948-2, FT-2968-2, FT-2974-2, FT-3027-2, FT-3052-2, FT-3062-2, FT-3073-2, FT-3093-2, FT-3128-2, FT-3197-2, FT-3216-2, FT-3352-2, FT-3386-2, FT-3422-2, FT-3489-2, FT-3512-2, FT-3515-2, FT-3548-2, FT-3564-2, FT-3687-2, FT-3703-2, FT-3801-2, FT-3852-2, FT-3872-2, FT-3873-2, FT-3881-2, FT-3883-2, FT-3886-2, FT-3893-2, FT-3897-2, FT-3907-2, FT-3908-2, FT-3934-2, FT-3935-2, FT-3937-2, FT-3938-2, FT-3941-2, FT-3951-2, FT-3954-2, FT-3959-2, FT-3963-2, FT-3967-2, FT-3985-2, FT-3999-2, FT-4001-1, and FT-4145-2.

In certain embodiments, the inhibitor is selected from the group consisting of: FT-1067, FT-2907, FT-3934, FT-3938, FT-3951, FT-3967, and FT-4001.

### BRIEF DESCRIPTION OF THE SEVERAL VIEWS OF THE DRAWINGS

FIG. 1 shows inhibitory dose response curves of FT-1055-3 for inhibiting luciferase activity of a β-catenin responsive reporter (pBARL) in both WNT stimulated A549/pBARL cells and TFG-β stimulated A549/pBARL cells.

FIG. 2 shows inhibitory dose response curves of FT-1067-3 for inhibiting luciferase activity of a β-catenin responsive reporter (pBARL) in both WNT stimulated A549/pBARL cells and TFG-β stimulated A549/pBARL cells.

FIG. 3 shows inhibitory dose response curves of FT-1069-1 for inhibiting luciferase activity of a β-catenin responsive reporter (pBARL) in both WNT stimulated A549/pBARL cells and TFG-β stimulated A549/pBARL cells.

FIG. 4 shows inhibitory dose response curves of FT-1083-1 for inhibiting luciferase activity of a β-catenin responsive reporter (pBARL) in both WNT stimulated A549/pBARL cells and TFG-β stimulated A549/pBARL cells.

FIG. 5 shows inhibitory dose response curves of FT-1147-3 for inhibiting luciferase activity of a β-catenin responsive reporter (pBARL) in both WNT stimulated A549/pBARL cells and TFG-β stimulated A549/pBARL cells.

FIG. 6 shows inhibitory dose response curves of FT-1150-3 for inhibiting luciferase activity of a β-catenin responsive reporter (pBARL) in both WNT stimulated A549/pBARL cells and TFG-β stimulated A549/pBARL cells.

FIG. 7 shows inhibitory dose response curves of FT-1202-1 for inhibiting luciferase activity of a β-catenin responsive reporter (pBARL) in both WNT stimulated A549/pBARL cells and TFG-β stimulated A549/pBARL cells.

FIG. 8 shows inhibitory dose response curves of FT-1203-1 for inhibiting luciferase activity of a β-catenin responsive reporter (pBARL) in both WNT stimulated A549/pBARL cells and TFG-β stimulated A549/pBARL cells.

FIG. 9 shows inhibitory dose response curves of FT-1812-4 for inhibiting luciferase activity of a β-catenin responsive reporter (pBARL) in both WNT stimulated A549/pBARL cells and TFG-β stimulated A549/pBARL cells.

FIG. 10 shows inhibitory dose response curves of FT-1265-1 for inhibiting luciferase activity of a β-catenin responsive reporter (pBARL) in both WNT stimulated A549/pBARL cells and TFG-β stimulated A549/pBARL cells.

FIG. 11 shows inhibitory dose response curves of FT-1281-1 for inhibiting luciferase activity of a β-catenin responsive reporter (pBARL) in both WNT stimulated A549/pBARL cells and TFG-β stimulated A549/pBARL cells.

FIG. 12 shows inhibitory dose response curves of FT-1294-5 for inhibiting luciferase activity of a β-catenin responsive reporter (pBARL) in both WNT stimulated A549/pBARL cells and TFG-β stimulated A549/pBARL cells.

FIG. 13 shows inhibitory dose response curves of FT-1301-1 for inhibiting luciferase activity of a β-catenin responsive reporter (pBARL) in both WNT stimulated A549/pBARL cells and TFG-β stimulated A549/pBARL cells.

FIG. 14 shows inhibitory dose response curves of FT-1320-1 for inhibiting luciferase activity of a β-catenin responsive reporter (pBARL) in both WNT stimulated A549/pBARL cells and TFG-β stimulated A549/pBARL cells.

FIG. 15 shows inhibitory dose response curves of FT-1355-2 for inhibiting luciferase activity of a β-catenin responsive reporter (pBARL) in both WNT stimulated A549/pBARL cells and TFG-β stimulated A549/pBARL cells.

FIG. 16 shows inhibitory dose response curves of FT-1361-2 for inhibiting luciferase activity of a β-catenin responsive reporter (pBARL) in both WNT stimulated A549/pBARL cells and TFG-β stimulated A549/pBARL cells.

FIG. 17 shows inhibitory dose response curves of FT-1366-2 for inhibiting luciferase activity of a β-catenin responsive reporter (pBARL) in both WNT stimulated A549/pBARL cells and TFG-β stimulated A549/pBARL cells.

FIG. 18 shows inhibitory dose response curves of FT-1398-2 for inhibiting luciferase activity of a β-catenin responsive reporter (pBARL) in both WNT stimulated A549/pBARL cells and TFG-β stimulated A549/pBARL cells.

FIG. 19 shows inhibitory dose response curves of FT-1434-2 for inhibiting luciferase activity of a β-catenin responsive reporter (pBARL) in both WNT stimulated A549/pBARL cells and TFG-β stimulated A549/pBARL cells.

FIG. 20 shows inhibitory dose response curves of FT-1435-2 for inhibiting luciferase activity of a β-catenin responsive reporter (pBARL) in both WNT stimulated A549/pBARL cells and TFG-β stimulated A549/pBARL cells.

FIG. 21 shows inhibitory dose response curves of FT-1436-1 for inhibiting luciferase activity of a β-catenin responsive reporter (pBARL) in both WNT stimulated A549/pBARL cells and TFG-β stimulated A549/pBARL cells.

FIG. 22 shows inhibitory dose response curves of FT-1480-1 for inhibiting luciferase activity of a β-catenin responsive reporter (pBARL) in both WNT stimulated A549/pBARL cells and TFG-β stimulated A549/pBARL cells.

FIG. 23 shows inhibitory dose response curves of FT-1497-1 for inhibiting luciferase activity of a β-catenin responsive reporter (pBARL) in both WNT stimulated A549/pBARL cells and TFG-β stimulated A549/pBARL cells.

FIG. 24 shows inhibitory dose response curves of FT-1504-3 for inhibiting luciferase activity of a β-catenin responsive reporter (pBARL) in both WNT stimulated A549/pBARL cells and TFG-β stimulated A549/pBARL cells.

FIG. 25 shows inhibitory dose response curves of FT-1515-1 for inhibiting luciferase activity of a β-catenin responsive reporter (pBARL) in both WNT stimulated A549/pBARL cells and TFG-β stimulated A549/pBARL cells.

FIG. 26 shows inhibitory dose response curves of FT-1517-1 for inhibiting luciferase activity of a β-catenin responsive reporter (pBARL) in both WNT stimulated A549/pBARL cells and TFG-β stimulated A549/pBARL cells.

FIG. 27 shows inhibitory dose response curves of FT-1518-1 for inhibiting luciferase activity of a β-catenin responsive reporter (pBARL) in both WNT stimulated A549/pBARL cells and TFG-β stimulated A549/pBARL cells.

FIG. 28 shows inhibitory dose response curves of FT-1532-1 for inhibiting luciferase activity of a β-catenin responsive reporter (pBARL) in both WNT stimulated A549/pBARL cells and TFG-β stimulated A549/pBARL cells.

FIG. 29 shows inhibitory dose response curves of FT-1575-2 for inhibiting luciferase activity of a β-catenin responsive reporter (pBARL) in both WNT stimulated A549/pBARL cells and TFG-β stimulated A549/pBARL cells.

FIG. 30 shows inhibitory dose response curves of FT-1609-1 for inhibiting luciferase activity of a β-catenin responsive reporter (pBARL) in both WNT stimulated A549/pBARL cells and TFG-β stimulated A549/pBARL cells.

FIG. 31 shows inhibitory dose response curves of FT-1612-3 for inhibiting luciferase activity of a β-catenin responsive reporter (pBARL) in both WNT stimulated A549/pBARL cells and TFG-β stimulated A549/pBARL cells.

FIG. 32 shows inhibitory dose response curves of FT-1613-1 for inhibiting luciferase activity of a β-catenin responsive reporter (pBARL) in both WNT stimulated A549/pBARL cells and TFG-β stimulated A549/pBARL cells.

FIG. 33 shows inhibitory dose response curves of FT-1660-1 for inhibiting luciferase activity of a β-catenin responsive reporter (pBARL) in both WNT stimulated A549/pBARL cells and TFG-β stimulated A549/pBARL cells.

FIG. 34 shows inhibitory dose response curves of FT-1678-1 for inhibiting luciferase activity of a β-catenin responsive reporter (pBARL) in both WNT stimulated A549/pBARL cells and TFG-β stimulated A549/pBARL cells.

FIG. 35 shows inhibitory dose response curves of FT-1688-1 for inhibiting luciferase activity of a β-catenin responsive reporter (pBARL) in both WNT stimulated A549/pBARL cells and TFG-β stimulated A549/pBARL cells.

FIG. 36 shows inhibitory dose response curves of FT-1693-1 for inhibiting luciferase activity of a β-catenin responsive reporter (pBARL) in both WNT stimulated A549/pBARL cells and TFG-β stimulated A549/pBARL cells.

FIG. 37 shows inhibitory dose response curves of FT-1812-3 for inhibiting luciferase activity of a β-catenin responsive reporter (pBARL) in both WNT stimulated A549/pBARL cells and TFG-β stimulated A549/pBARL cells.

FIG. 38 shows inhibitory dose response curves of FT-1915-2 for inhibiting luciferase activity of a β-catenin responsive reporter (pBARL) in both WNT stimulated A549/pBARL cells and TFG-β stimulated A549/pBARL cells.

FIG. 39 shows inhibitory dose response curves of FT-1986-3 for inhibiting luciferase activity of a β-catenin responsive reporter (pBARL) in both WNT stimulated A549/pBARL cells and TFG-β stimulated A549/pBARL cells.

FIG. 40 shows inhibitory dose response curves of FT-1992-3 for inhibiting luciferase activity of a β-catenin responsive reporter (pBARL) in both WNT stimulated A549/pBARL cells and TFG-β stimulated A549/pBARL cells.

FIG. 41 shows inhibitory dose response curves of FT-2014-2 for inhibiting luciferase activity of a β-catenin responsive reporter (pBARL) in both WNT stimulated A549/pBARL cells and TFG-β stimulated A549/pBARL cells.

FIG. 42 shows inhibitory dose response curves of FT-2046-2 for inhibiting luciferase activity of a β-catenin responsive reporter (pBARL) in both WNT stimulated A549/pBARL cells and TFG-β stimulated A549/pBARL cells.

FIG. 43 shows inhibitory dose response curves of FT-2051-2 for inhibiting luciferase activity of a β-catenin responsive reporter (pBARL) in both WNT stimulated A549/pBARL cells and TFG-β stimulated A549/pBARL cells.

FIG. 44 shows inhibitory dose response curves of FT-2081-2 for inhibiting luciferase activity of a β-catenin responsive reporter (pBARL) in both WNT stimulated A549/pBARL cells and TFG-β stimulated A549/pBARL cells.

FIG. 45 shows inhibitory dose response curves of FT-2103-2 for inhibiting luciferase activity of a β-catenin responsive reporter (pBARL) in both WNT stimulated A549/pBARL cells and TFG-β stimulated A549/pBARL cells.

FIG. 46 shows inhibitory dose response curves of FT-2115-2 for inhibiting luciferase activity of a β-catenin responsive reporter (pBARL) in both WNT stimulated A549/pBARL cells and TFG-β stimulated A549/pBARL cells.

FIG. 47 shows inhibitory dose response curves of FT-2228-3 for inhibiting luciferase activity of a β-catenin responsive reporter (pBARL) in both WNT stimulated A549/pBARL cells and TFG-β stimulated A549/pBARL cells.

FIG. 48 shows inhibitory dose response curves of FT-2254-2 for inhibiting luciferase activity of a β-catenin responsive reporter (pBARL) in both WNT stimulated A549/pBARL cells and TFG-β stimulated A549/pBARL cells.

FIG. 49 shows inhibitory dose response curves of FT-2318-2 for inhibiting luciferase activity of a β-catenin responsive reporter (pBARL) in both WNT stimulated A549/pBARL cells and TFG-β stimulated A549/pBARL cells.

FIG. 50 shows inhibitory dose response curves of FT-2342-2 for inhibiting luciferase activity of a β-catenin responsive reporter (pBARL) in both WNT stimulated A549/pBARL cells and TFG-β stimulated A549/pBARL cells.

FIG. 51 shows inhibitory dose response curves of FT-2474-2 for inhibiting luciferase activity of a β-catenin responsive reporter (pBARL) in both WNT stimulated A549/pBARL cells and TFG-β stimulated A549/pBARL cells.

FIG. 52 shows inhibitory dose response curves of FT-2498-2 for inhibiting luciferase activity of a β-catenin responsive reporter (pBARL) in both WNT stimulated A549/pBARL cells and TFG-β stimulated A549/pBARL cells.

FIG. 53 shows inhibitory dose response curves of FT-2562-3 for inhibiting luciferase activity of a β-catenin responsive reporter (pBARL) in both WNT stimulated A549/pBARL cells and TFG-β stimulated A549/pBARL cells.

FIG. 54 shows inhibitory dose response curves of FT-2580-2 for inhibiting luciferase activity of a β-catenin responsive reporter (pBARL) in both WNT stimulated A549/pBARL cells and TFG-β stimulated A549/pBARL cells.

FIG. 55 shows inhibitory dose response curves of FT-2619-2 for inhibiting luciferase activity of a β-catenin responsive reporter (pBARL) in both WNT stimulated A549/pBARL cells and TFG-β stimulated A549/pBARL cells.

FIG. 56 shows inhibitory dose response curves of FT-2633-2 for inhibiting luciferase activity of a β-catenin responsive reporter (pBARL) in both WNT stimulated A549/pBARL cells and TFG-β stimulated A549/pBARL cells.

FIG. 57 shows inhibitory dose response curves of FT-2660-2 for inhibiting luciferase activity of a β-catenin responsive reporter (pBARL) in both WNT stimulated A549/pBARL cells and TFG-β stimulated A549/pBARL cells.

FIG. 58 shows inhibitory dose response curves of FT-2691-2 for inhibiting luciferase activity of a β-catenin responsive reporter (pBARL) in both WNT stimulated A549/pBARL cells and TFG-β stimulated A549/pBARL cells.

FIG. 59 shows inhibitory dose response curves of FT-2693-3 for inhibiting luciferase activity of a β-catenin responsive reporter (pBARL) in both WNT stimulated A549/pBARL cells and TFG-β stimulated A549/pBARL cells.

FIG. 60 shows inhibitory dose response curves of FT-2770-2 for inhibiting luciferase activity of a β-catenin responsive reporter (pBARL) in both WNT stimulated A549/pBARL cells and TFG-β stimulated A549/pBARL cells.

FIG. 61 shows inhibitory dose response curves of FT-2820-2 for inhibiting luciferase activity of a β-catenin responsive reporter (pBARL) in both WNT stimulated A549/pBARL cells and TFG-β stimulated A549/pBARL cells.

FIG. 62 shows inhibitory dose response curves of FT-2862-2 for inhibiting luciferase activity of a β-catenin responsive reporter (pBARL) in both WNT stimulated A549/pBARL cells and TFG-β stimulated A549/pBARL cells.

FIG. 63 shows inhibitory dose response curves of FT-2863-2 for inhibiting luciferase activity of a β-catenin responsive reporter (pBARL) in both WNT stimulated A549/pBARL cells and TFG-β stimulated A549/pBARL cells.

FIG. 64 shows inhibitory dose response curves of FT-2907-2 for inhibiting luciferase activity of a β-catenin responsive reporter (pBARL) in both WNT stimulated A549/pBARL cells and TFG-β stimulated A549/pBARL cells.

FIG. 65 shows inhibitory dose response curves of FT-2909-2 for inhibiting luciferase activity of a β-catenin responsive reporter (pBARL) in both WNT stimulated A549/pBARL cells and TFG-β stimulated A549/pBARL cells.

FIG. 66 shows inhibitory dose response curves of FT-2912-3 for inhibiting luciferase activity of a β-catenin responsive reporter (pBARL) in both WNT stimulated A549/pBARL cells and TFG-β stimulated A549/pBARL cells.

FIG. 67 shows inhibitory dose response curves of FT-2920-3 for inhibiting luciferase activity of a β-catenin responsive reporter (pBARL) in both WNT stimulated A549/pBARL cells and TFG-β stimulated A549/pBARL cells.

FIG. 68 shows inhibitory dose response curves of FT-2947-2 for inhibiting luciferase activity of a β-catenin responsive reporter (pBARL) in both WNT stimulated A549/pBARL cells and TFG-β stimulated A549/pBARL cells.

FIG. 69 shows inhibitory dose response curves of FT-2948-2 for inhibiting luciferase activity of a β-catenin responsive reporter (pBARL) in both WNT stimulated A549/pBARL cells and TFG-β stimulated A549/pBARL cells.

FIG. 70 shows inhibitory dose response curves of FT-2968-2 for inhibiting luciferase activity of a β-catenin responsive reporter (pBARL) in both WNT stimulated A549/pBARL cells and TFG-β stimulated A549/pBARL cells.

FIG. 71 shows inhibitory dose response curves of FT-2974-2 for inhibiting luciferase activity of a β-catenin responsive reporter (pBARL) in both WNT stimulated A549/pBARL cells and TFG-β stimulated A549/pBARL cells.

FIG. 72 shows inhibitory dose response curves of FT-3027-2 for inhibiting luciferase activity of a β-catenin responsive reporter (pBARL) in both WNT stimulated A549/pBARL cells and TFG-β stimulated A549/pBARL cells.

FIG. 73 shows inhibitory dose response curves of FT-3052-2 for inhibiting luciferase activity of a β-catenin responsive reporter (pBARL) in both WNT stimulated A549/pBARL cells and TFG-β stimulated A549/pBARL cells.

FIG. 74 shows inhibitory dose response curves of FT-3062-2 for inhibiting luciferase activity of a β-catenin responsive reporter (pBARL) in both WNT stimulated A549/pBARL cells and TFG-β stimulated A549/pBARL cells.

FIG. 75 shows inhibitory dose response curves of FT-3073-2 for inhibiting luciferase activity of a β-catenin responsive reporter (pBARL) in both WNT stimulated A549/pBARL cells and TFG-β stimulated A549/pBARL cells.

FIG. 76 shows inhibitory dose response curves of FT-3093-2 for inhibiting luciferase activity of a β-catenin responsive reporter (pBARL) in both WNT stimulated A549/pBARL cells and TFG-β stimulated A549/pBARL cells.

FIG. 77 shows inhibitory dose response curves of FT-3128-2 for inhibiting luciferase activity of a β-catenin responsive reporter (pBARL) in both WNT stimulated A549/pBARL cells and TFG-β stimulated A549/pBARL cells.

FIG. 78 shows inhibitory dose response curves of FT-3197-2 for inhibiting luciferase activity of a β-catenin responsive reporter (pBARL) in both WNT stimulated A549/pBARL cells and TFG-β stimulated A549/pBARL cells.

FIG. 79 shows inhibitory dose response curves of FT-3216-2 for inhibiting luciferase activity of a β-catenin responsive reporter (pBARL) in both WNT stimulated A549/pBARL cells and TFG-β stimulated A549/pBARL cells.

FIG. 80 shows inhibitory dose response curves of FT-3352-2 for inhibiting luciferase activity of a β-catenin responsive reporter (pBARL) in both WNT stimulated A549/pBARL cells and TFG-β stimulated A549/pBARL cells.

FIG. 81 shows inhibitory dose response curves of FT-3386-2 for inhibiting luciferase activity of a β-catenin responsive reporter (pBARL) in both WNT stimulated A549/pBARL cells and TFG-β stimulated A549/pBARL cells.

FIG. 82 shows inhibitory dose response curves of FT-3422-2 for inhibiting luciferase activity of a β-catenin responsive reporter (pBARL) in both WNT stimulated A549/pBARL cells and TFG-β stimulated A549/pBARL cells.

FIG. 83 shows inhibitory dose response curves of FT-3489-2 for inhibiting luciferase activity of a β-catenin responsive reporter (pBARL) in both WNT stimulated A549/pBARL cells and TFG-β stimulated A549/pBARL cells.

FIG. 84 shows inhibitory dose response curves of FT-3512-2 for inhibiting luciferase activity of a β-catenin responsive reporter (pBARL) in both WNT stimulated A549/pBARL cells and TFG-β stimulated A549/pBARL cells.

FIG. 85 shows inhibitory dose response curves of FT-3515-2 for inhibiting luciferase activity of a β-catenin responsive reporter (pBARL) in both WNT stimulated A549/pBARL cells and TFG-β stimulated A549/pBARL cells.

FIG. 86 shows inhibitory dose response curves of FT-3548-2 for inhibiting luciferase activity of a β-catenin responsive reporter (pBARL) in both WNT stimulated A549/pBARL cells and TFG-β stimulated A549/pBARL cells.

FIG. 87 shows inhibitory dose response curves of FT-3564-2 for inhibiting luciferase activity of a β-catenin responsive reporter (pBARL) in both WNT stimulated A549/pBARL cells and TFG-β stimulated A549/pBARL cells.

FIG. 88 shows inhibitory dose response curves of FT-3687-2 for inhibiting luciferase activity of a β-catenin responsive reporter (pBARL) in both WNT stimulated A549/pBARL cells and TFG-β stimulated A549/pBARL cells.

FIG. 89 shows inhibitory dose response curves of FT-3703-2 for inhibiting luciferase activity of a β-catenin responsive reporter (pBARL) in both WNT stimulated A549/pBARL cells and TFG-β stimulated A549/pBARL cells.

FIG. 90 shows inhibitory dose response curves of FT-3801-2 for inhibiting luciferase activity of a β-catenin responsive reporter (pBARL) in both WNT stimulated A549/pBARL cells and TFG-β stimulated A549/pBARL cells.

FIG. 91 shows inhibitory dose response curves of FT-3852-2 for inhibiting luciferase activity of a β-catenin responsive reporter (pBARL) in both WNT stimulated A549/pBARL cells and TFG-β stimulated A549/pBARL cells.

FIG. 92 shows inhibitory dose response curves of FT-3872-2 for inhibiting luciferase activity of a β-catenin responsive reporter (pBARL) in both WNT stimulated A549/pBARL cells and TFG-β stimulated A549/pBARL cells.

FIG. 93 shows inhibitory dose response curves of FT-3873-2 for inhibiting luciferase activity of a β-catenin responsive reporter (pBARL) in both WNT stimulated A549/pBARL cells and TFG-β stimulated A549/pBARL cells.

FIG. 94 shows inhibitory dose response curves of FT-3881-2 for inhibiting luciferase activity of a β-catenin responsive reporter (pBARL) in both WNT stimulated A549/pBARL cells and TFG-β stimulated A549/pBARL cells.

FIG. 95 shows inhibitory dose response curves of FT-3883-2 for inhibiting luciferase activity of a β-catenin responsive reporter (pBARL) in both WNT stimulated A549/pBARL cells and TFG-β stimulated A549/pBARL cells.

FIG. 96 shows inhibitory dose response curves of FT-3886-2 for inhibiting luciferase activity of a β-catenin responsive reporter (pBARL) in both WNT stimulated A549/pBARL cells and TFG-β stimulated A549/pBARL cells.

FIG. 97 shows inhibitory dose response curves of FT-3893-2 for inhibiting luciferase activity of a β-catenin responsive reporter (pBARL) in both WNT stimulated A549/pBARL cells and TFG-β stimulated A549/pBARL cells.

FIG. 98 shows inhibitory dose response curves of FT-3897-2 for inhibiting luciferase activity of a β-catenin responsive reporter (pBARL) in both WNT stimulated A549/pBARL cells and TFG-β stimulated A549/pBARL cells.

FIG. 99 shows inhibitory dose response curves of FT-3907-2 for inhibiting luciferase activity of a β-catenin responsive reporter (pBARL) in both WNT stimulated A549/pBARL cells and TFG-β stimulated A549/pBARL cells.

FIG. 100 shows inhibitory dose response curves of FT-3908-2 for inhibiting luciferase activity of a β-catenin responsive reporter (pBARL) in both WNT stimulated A549/pBARL cells and TFG-β stimulated A549/pBARL cells.

FIG. 101 shows inhibitory dose response curves of FT-3934-2 for inhibiting luciferase activity of a β-catenin responsive reporter (pBARL) in both WNT stimulated A549/pBARL cells and TFG-β stimulated A549/pBARL cells.

FIG. 102 shows inhibitory dose response curves of FT-3935-2 for inhibiting luciferase activity of a β-catenin responsive reporter (pBARL) in both WNT stimulated A549/pBARL cells and TFG-β stimulated A549/pBARL cells.

FIG. 103 shows inhibitory dose response curves of FT-3937-2 for inhibiting luciferase activity of a β-catenin responsive reporter (pBARL) in both WNT stimulated A549/pBARL cells and TFG-β stimulated A549/pBARL cells.

FIG. 104 shows inhibitory dose response curves of FT-3938-2 for inhibiting luciferase activity of a β-catenin responsive reporter (pBARL) in both WNT stimulated A549/pBARL cells and TFG-β stimulated A549/pBARL cells.

FIG. 105 shows inhibitory dose response curves of FT-3941-2 for inhibiting luciferase activity of a β-catenin responsive reporter (pBARL) in both WNT stimulated A549/pBARL cells and TFG-β stimulated A549/pBARL cells.

FIG. 106 shows inhibitory dose response curves of FT-3951-2 for inhibiting luciferase activity of a β-catenin responsive reporter (pBARL) in both WNT stimulated A549/pBARL cells and TFG-β stimulated A549/pBARL cells.

FIG. 107 shows inhibitory dose response curves of FT-3954-2 for inhibiting luciferase activity of a β-catenin responsive reporter (pBARL) in both WNT stimulated A549/pBARL cells and TFG-β stimulated A549/pBARL cells.

FIG. 108 shows inhibitory dose response curves of FT-3959-2 for inhibiting luciferase activity of a β-catenin responsive reporter (pBARL) in both WNT stimulated A549/pBARL cells and TFG-β stimulated A549/pBARL cells.

FIG. 109 shows inhibitory dose response curves of FT-3963-2 for inhibiting luciferase activity of a β-catenin responsive reporter (pBARL) in both WNT stimulated A549/pBARL cells and TFG-β stimulated A549/pBARL cells.

FIG. 110 shows inhibitory dose response curves of FT-3967-2 for inhibiting luciferase activity of a β-catenin responsive reporter (pBARL) in both WNT stimulated A549/pBARL cells and TFG-β stimulated A549/pBARL cells.

FIG. 111 shows inhibitory dose response curves of FT-3985-2 for inhibiting luciferase activity of a β-catenin responsive reporter (pBARL) in both WNT stimulated A549/pBARL cells and TFG-β stimulated A549/pBARL cells.

FIG. 112 shows inhibitory dose response curves of FT-3999-2 for inhibiting luciferase activity of a β-catenin responsive reporter (pBARL) in both WNT stimulated A549/pBARL cells and TFG-β stimulated A549/pBARL cells.

FIG. 113 shows inhibitory dose response curves of FT-4001-1 for inhibiting luciferase activity of a β-catenin responsive reporter (pBARL) in both WNT stimulated A549/pBARL cells and TFG-β stimulated A549/pBARL cells.

FIG. 114 shows inhibitory dose response curves of FT-4145-2 for inhibiting luciferase activity of a β-catenin responsive reporter (pBARL) in both WNT stimulated A549/pBARL cells and TFG-β stimulated A549/pBARL cells.

FIG. 115 shows the results of a western blot experiment. Mouse alveolar type II cells were cultured on fibronectin to induce EMT. Induced cells were treated with either a 1:2000 dilution of DMSO (a negative control), 5 uM FT-2097, 5 uM FT-3934, 5 uM FT-4001 or 5 uM SB431542 (a positive control that inhibits TGF-β signaling). At the end of the treatment, cell lysates were prepared and resolved on an SDS gel. Western blotting was performed with anti-SMA and GAPDH antibodies.

### DETAILED DESCRIPTION

### A. Overview

Although many different types of tissues and organs can develop fibrosis and/or fibroproliferative disease, the accumulation of fibrotic material at various tissues and organs can occur by common mechanisms including, but not limited to, increased EMT/EnMT, prolonged myofibroblast activation, and increased deposition of extracellular matrix.

As used herein, the term “epithelial to mesenchymal transition” (EMT) refers to the conversion of a cell from an epithelial to a mesenchymal phenotype, which is a normal process of embryonic development. EMT is also the process whereby injured epithelial cells that function as ion and fluid transporters become matrix remodeling mesenchymal cells. The criteria for defining EMT in vitro involve the loss of epithelial cell polarity, the separation into individual cells and subsequent dispersion after the acquisition of cell motility (see Vincent-Salomon et al., Breast Cancer Res. 2003; 5(2): 101-106). Growth factors including, but not limited to, TGF-β (e.g., TGF-β1, TGF-β2, TGF-β3 and Wnts (e.g., Wnt1, Wnt3A, Wnt8, Wnt10a) and transcription factors including, but not limited to, LEF and β-catenin are causally involved in regulating EMT (see Thompson et al., Cancer Research 65, 5991-5995, Jul. 15, 2005).

As used herein, the term “endothelial to mesenchymal transition” (EnMT) refers to the phenotypic conversion of endothelial cells to a mesenchymal-myofibroblast phenotype.

As used herein, the term “epithelium” refers to the covering of internal and external surfaces of the body, including the lining of vessels and other small cavities. It consists of a collection of epithelial cells forming a relatively thin sheet or layer due to the constituent cells being mutually and extensively adherent laterally by cell-to-cell junctions. The layer is polarized and has apical and basal sides. Despite the tight regimentation of the epithelial cells the epithelium does have some plasticity and cells in an epithelial layer can alter shape, such as change from flat to columnar, or pinch in at one end and expand at the other. However, these tend to occur in cell groups rather than individually (see Thompson et al., Cancer Research 65, 5991-5995, Jul. 15, 2005).

As used herein, the term “mesenchyme” refers to the part of the embryonic mesoderm, consisting of loosely packed, unspecialized cells set in a gelatinous ground substance, from which connective tissue, bone, cartilage, and the circulatory and lymphatic systems develop. Mesenchyme is a collection of cells which form a relatively diffuse tissue network. Mesenchyme is not a complete cellular layer and the cells typically have only points on their surface engaged in adhesion to their neighbors. These adhesions may also involve cadherin associations (see Thompson et al., Cancer Research 65, 5991-5995, Jul. 15, 2005).

As used herein, the term “myofibroblast” refers to fibroblasts that are associated with the increased and often pathological deposition of ECM at fibrotic lesions. Myofibroblasts are activated in response to injury or increased epithelial to mesenchymal crosstalk and are thought to be the primary producers of ECM components following injury. Myofibroblasts originate from differentiation of resident mesenchymal fibroblasts (hepatic stellate cells in the liver), from EMT, and from EnMT. Myofibroblast differentiation is an early event in the development of fibrosis. Myofibroblast-like cells express smooth muscle (SM) cytoskeletal markers (α-SM actin in particular) and participate actively in the production of extracellular matrix.

Increased EMT/EnMT, prolonged myofibroblast activation, and increased deposition of extracellular matrix are features common to many fibroproliferative diseases, including but not limited to pulmonary fibrosis, liver fibrosis, kidney fibrosis, systemic sclerosis, and fibrosis arising from transplant rejection. The spectrum of affected organs, the usually progressive nature of the fibrotic process, the large number of affected persons, and the absence of effective treatment pose an enormous challenge when treating fibrotic diseases. Current treatments for fibrotic diseases typically target the inflammatory response, but there is accumulating evidence that the mechanisms driving fibrosis are distinct from those regulating inflammation. In fact, some studies suggest that ongoing inflammation reverses established and progressive fibrosis.

Accordingly, the present invention contemplates, in part, to provide compositions comprising inhibitors of cell signaling pathways that underlie the mechanism(s) of fibrosis that are common to most tissues, including without limitation, cell signaling pathways associated with EMT/EnMT, myofibroblast activation, and myofibroblast deposition of extracellular matrix. Without wishing to be bound to any particular theory, it is contemplated that fibrosis and fibroproliferative diseases can be more effectively prevented, reversed, treated, or ameliorated by inhibiting multiple signaling pathways associated with fibrosis.

Thus, by providing compositions that more effectively inhibit cell signaling pathways associated with EMT/EnMT, myofibroblast activation, and myofibroblast deposition of extracellular matrix, the present invention provides a much needed solution to a pandemic heath care crisis.

### B. Fibrosis and Fibroproliferative Disease

Although many different types of tissues and organs can develop fibrosis and/or fibroproliferative disease, the accumulation of fibrotic material at various tissues and organs can occur by a common mechanism.

As used herein, the term “fibrosis” refers to the formation or development of excess fibrous connective tissue in an organ or tissue as a reparative or reactive process, as opposed to a formation of fibrous tissue as a normal constituent of an organ or tissue. Fibrosis can be either chronic or acute. Fibrotic conditions include excessive amounts of fibrous tissue, including excessive amounts of extracellular matrix accumulation within a tissue, forming tissue which causes dysfunction and, potentially, organ failure. Chronic fibrosis includes fibrosis of the major organs, most commonly lung, liver, kidney and/or heart. Acute fibrosis (usually with a sudden and severe onset and of short duration) occurs typically as a common response to various forms of trauma including injuries, ischemic illness (e.g., cardiac scarring following heart attack), environmental pollutants, alcohol and other types of toxins, acute respiratory distress syndrome, radiation and chemotherapy treatments. All tissues damaged by trauma can become fibrotic, particularly if the damage is repeated.

As used herein, the term “interstitial fibrosis” refers to fibrosis relating to or situated in the small, narrow spaces between tissues or parts of an organ. For example, interstitial pulmonary fibrosis (also known as interstitial lung disease and pulmonary fibrosis) refers to fibrosis (i.e., scarring) of the interstitium, i.e., the tissue between the air sacs of the lungs. Additionally, renal interstitial fibrosis (also known as kidney fibrosis) is characterized by the destruction of renal tubules and interstitial capillaries as well as by the accumulation of extracellular matrix proteins. As used herein, the term “vascular remodeling” is a type of fibrosis that refers to the active process of structural and cellular changes in the vasculature. All of these changes are characterized by an increased number of cells which express alpha-smooth muscle actin. This accumulation of alpha-smooth muscle positive cells could result from the proliferative expansion of resident vascular smooth muscle cells (SMC), recruitment of circulating progenitor cells to sites of vascular injury, or transition of endothelial cells towards a mesenchymal phenotype (EnMT).III.

As used herein, the terms “fibrotic disease” or “fibroproliferative disease” are used interchangeably and refer to diseases that include those mentioned herein, and further include acute and chronic, clinical or sub-clinical presentation, in which fibrogenic associated biology or pathology is evident. Fibroproliferative diseases are characterized by increased EMT/EnMT, prolonged myofibroblast activation, and excessive deposition of ECM. Fibroproliferative disease is responsible for morbidity and mortality associated with vascular diseases, such as cardiac disease, cerebral disease, and peripheral vascular disease, and with organ failure in a variety of chronic diseases affecting the pulmonary system, renal system, eyes, cardiac system, hepatic system, digestive system, and skin (Wynn, Nature Reviews. 2004; 4:583-594). However, to date, there are no therapies on the market that are effective in treating or preventing fibrotic disease

Exemplary fibroproliferative diseases include, but are not limited to, scleroderma (including morphea, generalized morphea, or linear scleroderma), kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproliferative syndrome, gynecological cancer, Kaposi's sarcoma, Hansen's disease, collagenous colitis, acute fibrosis, systemic sclerosis, and fibrosis arising from tissue or organ transplant or graft rejection.

### C. Methods of Treatment

The present invention contemplates, in part, to provide methods of preventing, reversing, treating, and/or ameliorating fibrosis or fibroproliferative disease in a subject.

As used herein, the terms “subject,” “subject in need of treatment,” and “subject in need thereof,” are to be used interchangeably and refer to any mammal, including humans, domestic and farm animals, and zoo, sports, and pet animals, such as dogs, horses, cats, sheep, pigs, goats, cows, rats, mice, etc. that is in need of treatment for one or more fibroproliferative diseases. The preferred mammal herein is a human, including adults, children, and the elderly.

In one particular embodiment, a subject has an accumulation of fibrotic tissue, scar tissue, and/or extracellular matrix material (e.g., collagen, vimentin, actin, fibronectin, etc.) on or within one or more tissues or organs within the body. In another particular embodiment, a subject has received a clinical diagnosis of one or more fibrosis conditions.

In a certain embodiment, a subject exhibits one or more symptoms of a fibrosis condition (Khalil and O'Connor, Canadian Medical Journal. 2004; 777:153-160). For example, a subject can exhibit one or more symptoms of a fibroproliferative disease of the liver (e.g., liver tissue injury or scarring cause by, e.g., viral hepatitis, alcohol abuse, drugs, metabolic diseases due to overload of iron or copper, autoimmune attack of hepatocytes or bile duct epithelium, or congenital abnormalities) (Friedman, J. Biol. Chem. 2000; 275:2247-2250); a fibroproliferative disease of the lung (e.g., lung tissue injury or scarring caused by or related to an inflammatory response of the lung to an inciting event, including e.g., idiopathic pulmonary fibrosis) (Garantziotis et al., J. Clin. Invest. 2004; 114:319-321); scleroderma of the skin or other organ(s) (Trojanowska, Frontiers Biosci.; 2002; 7:d608-618); and/or a fibroproliferative disease of the kidney (e.g., kidney tissue injury or scarring related to glomerulosclerosis or tubular interstitial fibrosis) (Negri, J. Nephrol.; 2004; 17:496-503).

The terms “treat,” “treating,” and “treatment,” as used herein, refer to therapeutic or preventative measures described herein. The methods of “treatment” include administration of one or more small molecule inhibitors of Wnt- and TGF-β-mediated β-catenin signaling to a subject in order to inhibit, prevent, reverse (cure), delay, reduce the severity of, reduce the progression of or ameliorate one or more symptoms of fibrosis or a fibroproliferative disease, in order to improve the quality of life and prolong the survival of a subject beyond that expected in the absence of such treatment. The efficacy of treatment ranges from amelioration of symptoms to complete reversal of fibrosis or a fibroproliferative disease. The efficacy of treatment and progress thereof may be measured by performing organ function tests, as routinely practice in the art.

In other various embodiments, the present invention provides, in part, methods to inhibit or reduce EMT/EnMT in a cell and to reduce myofibroblast activation.

In one embodiment, a method of inhibiting epithelial to mesenchymal transition (EMT) in an epithelial cell comprises contacting the epithelial cell with an inhibitor of β-catenin signaling, wherein the inhibitor inhibits Wnt- and TGF-β-mediated β-catenin signaling. Exemplary epithelial cells that can be used with the present invention include epithelial cells obtained from the lung, the gut, the skin, the eye, the kidney, and the liver. In preferred embodiments, epithelial cells are selected from the group consisting of a lung cell, a kidney cell, or a liver cell.

Without wishing to be bound to any particular theory, the present invention contemplates that inhibiting EMT will result in less myofibroblasts, less activation of myofibroblasts, and less ECM deposition in fibrosis or fibroproliferative disease compared to an epithelial cell that undergoes EMT in fibrosis or fibroproliferative disease.

In a particular embodiment, a method of inhibiting endothelial to mesenchymal transition (EnMT) in an endothelial cell comprises contacting the endothelial cell with an inhibitor of β-catenin signaling, wherein the inhibitor inhibits Wnt- and TGF-β-mediated β-catenin signaling. Exemplary endothelial cells that can be used with the present invention include endothelial cells obtained from the lung, the gut, the skin, the pancreas, the kidney, and the liver. In preferred embodiments, endothelial cells are selected from the group consisting of a lung cell, a kidney cell, or a liver cell.

Without wishing to be bound to any particular theory, the present invention contemplates that inhibiting EnMT will result in less myofibroblasts, less activation of myofibroblasts, and less ECM deposition in fibrosis or fibroproliferative disease compared to an epithelial cell that undergoes EnMT in fibrosis or fibroproliferative disease.

As discussed herein throughout, an important cellular mediator of fibrosis is the myofibroblast, which when activated serves as a primary collagen-producing cell in fibrotic lesions. Myofibroblasts can be generated from a variety of sources including resident mesenchymal cells, epithelial cells, and endothelial cells in processes termed epithelial/endothelial-mesenchymal (EMT/EnMT) transition, as well as from circulating fibroblast-like cells called fibrocytes that are derived from bone-marrow stem cells. Myofibroblasts are activated by a variety of mechanisms, including paracrine signals derived from epithelial cells, known as epithelial-mesenchymal interactions; and autocrine factors secreted by myofibroblasts among other mechanisms.

In one embodiment, the present invention provides a method of inhibiting myofibroblast activation in a myofibroblast comprising contacting the myofibroblast with an inhibitor of β-catenin signaling, wherein the inhibitor inhibits Wnt- and TGF-β-mediated β-catenin signaling. Exemplary myofibroblasts that can be used with the present invention include those obtained from the lung, the gut, the skin, the pancreas, the eye, the kidney, and the liver. In preferred embodiments, myofibroblasts are obtained from or located in a lung tissue, a kidney tissue, or a liver tissue.

Without wishing to be bound to any particular theory, the present invention contemplates that inhibiting myofibroblast activation results in less ECM deposition in fibrosis or fibroproliferative disease, than when myofibroblast activation is not inhibited.

In various embodiments, the present invention contemplates, in part, a method of preventing, reversing, treating, and/or ameliorating fibrosis in a subject comprising administering one or more inhibitors of β-catenin signaling to the subject, wherein the inhibitor inhibits or reduces Wnt- and TGFβ-mediated β-catenin signaling. Exemplary fibroproliferative diseases that can be treated using the methods of the present invention include pulmonary fibrosis, liver fibrosis, kidney fibrosis, systemic sclerosis, and fibrosis due to transplant rejection.

1. Lung Fibrosis

In various embodiments, the present invention contemplates, in part, a method of preventing, reversing, treating, and/or ameliorating lung fibrosis in a subject comprising administering one or more inhibitors of β-catenin signaling to the subject, wherein the inhibitor inhibits or reduces Wnt- and TGFβ-mediated β-catenin signaling.

A subject in need of treatment for lung fibrosis with small molecule inhibitors of the present invention includes subjects with pulmonary fibrosis, particularly early stage pulmonary fibrosis, and subjects at risk of pulmonary fibrosis. Subjects suffering from pulmonary fibrosis include subjects suffering from idiopathic pulmonary fibrosis, sarcoidosis, familial pulmonary fibrosis, pulmonary fibrosis associated with collagen-vascular disorders or vasculitides, histiocytosis X, Goodpasture's syndrome, chronic eosinophilic pneumonia, idiopathic pulmonary hemosiderosis, hypersensitivity pneumonitides; subjects suffering from pulmonary fibrosis caused by inhalation of organic or inorganic dusts, such as coal, crystalline silica and silicates such as asbestos (causing, e.g., silicosis, asbestosis, coal worker's or carbon pneumoconiosis); subjects suffering from pulmonary fibrosis caused by exposure to radiation or toxic agents such as paraquat, caused by an infectious agent, caused by inhalation of noxious gases, aerosols, chemical dusts, fumes or vapors, or drug-induced interstitial lung disease (ILD). Subjects at risk of pulmonary fibrotic disease include subjects undergoing radiation therapy or chemotherapy; subjects with a family history of or genetic factors indicating a predisposition to ILD; subjects in occupations involving exposure to radiation, toxic agents, or inhalation of dusts or noxious vapors; and subjects suffering from infections that may lead to complications that include pulmonary fibrosis. Subjects suffering from pulmonary fibrosis also include subjects suffering from secondary fibrosis, which may be brought on by an inflammatory condition, such as sarcoidosis, rheumatoid arthritis, systemic sclerosis, scleroderma, extrinsic allergic alveolitis, severe asthma, systemic granulomatosis vasculitis and/or adult respiratory distress syndrome (ARDS).

Lung or pulmonary fibrosis is a common feature of many lung diseases, such as idiopathic pulmonary fibrosis, adult respiratory distress syndrome, fibrosis with collagen vascular disease, bronchiolitis obliterans, respiratory bronchiolitis, sarcoidosis, histiocytosis X, Hermansky-Pudlak syndrome, nonspecific interstitial pneumonia, acute interstitial pneumonia, lymphocytic interstitial pneumonia, and cryptogenic organizing pneumonia. Signs or clinical symptoms of lung fibrosis include, e.g., increased deposition of collagen, particularly in alveolar septa and peribronchial parenchyma, thickened alveolar septa, decreased gas exchange resulting in elevated circulating carbon dioxide and reduced circulating oxygen levels, decreased lung elasticity which can manifest as restrictive lung functional impairment with decreased lung volumes and compliance on pulmonary function tests, bilateral reticulonodular images on chest X-ray, progressive dyspnea (difficulty breathing), and hypoxemia at rest that worsens with exercise. Lung fibrosis associated with any of these diseases may comprise increased EMT, prolonged myofibroblast activation, and increased or exaggerated ECM deposition in the cell interstitium, or other signs or clinical symptoms associated with lung fibrosis.

Lung fibrosis is recognized as a problem of increased EMT, prolonged myofibroblast activation, and excessive extracellular matrix production by fibroblasts in the lung. The responsible fibroblasts can be resident or created from the EMT of lung epithelial cells, e.g., ATII cells.

In one embodiment, the present invention provides methods to treat a subject that has or is at risk of having Idiopathic Pulmonary Fibrosis (IPF). IPF is characterized by excessive synthesis of extracellular matrix by 1) resident fibroblasts (myofibroblasts) in the lung and 2) by myofibroblasts differentiated from lung epithelial cells in a process called epithelial mesenchymal transition (EMT) (Scotton and Chambers, Chest. 2007; 132, 1311-1321; Willis et al., Am J. Pathol. 2005; 166(5), 1321-1332). The canonical Wnt pathway promotes epithelial to mesenchymal transition (EMT), myofibroblast activation, and increased extracellular matrix synthesis in various forms of fibrosis and fibroproliferative disease, e.g., idiopathic pulmonary fibrosis (Chilosi et al., Am J. Pathol. 2003; 162(5), 1495-1502; Königshoff et al. J Clin Invest. 2009; 119(4), 772-787). In addition, experimental evidence supports the notion that the TGF-β pathway induces EMT in lung epithelium; that elevated TGF-β signaling is present in models of lung fibrosis; and that TGF-β induced fibrosis in IPF is mediated through β-catenin signaling (Scotton and Chambers, 2007; Kim et al., J Clin Invest. 2009; 119(1), 213-224). β-catenin is well recognized as the mediator of canonical Wnt signaling. However, TGF-β signaling in fibrosis induces phosphorylation of tyrosine 654 (Y654) on β-catenin. Y654 phosphorylation of β-catenin has not been reported in Wnt-mediated β-catenin signaling.

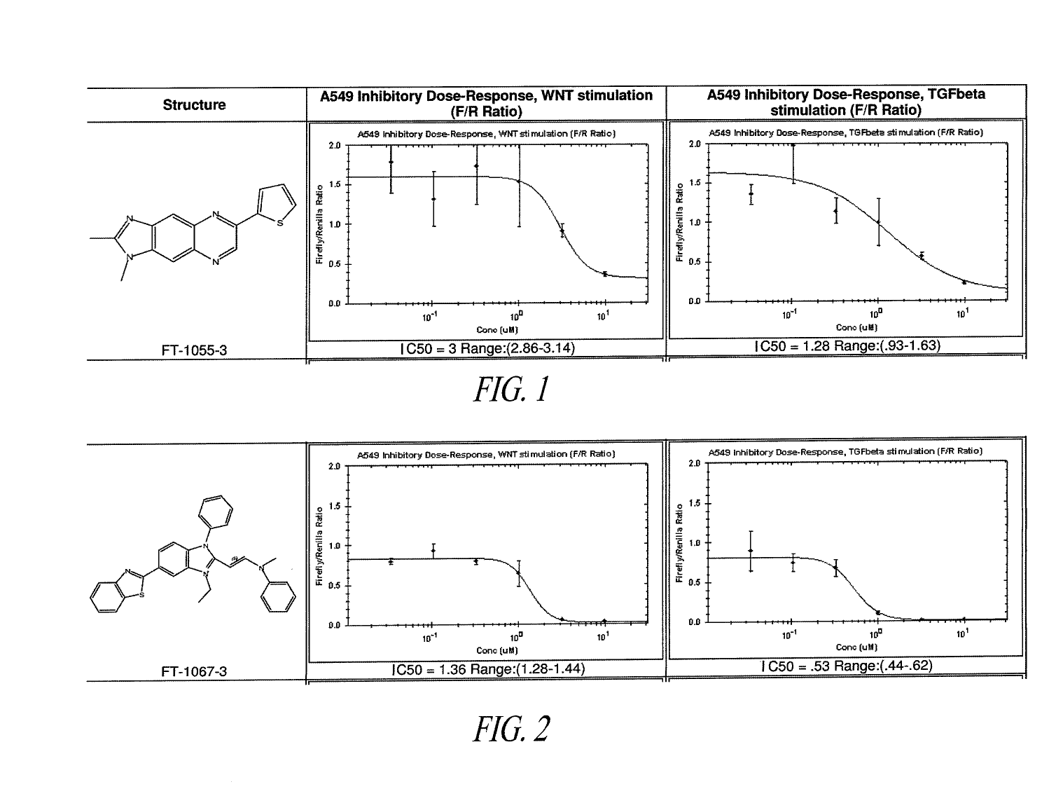

Without wishing to be bound by any particular theory, the present invention contemplates, in part, that small molecules identified as inhibitors of both Wnt- and TGFβ-mediated β-catenin signaling provide a more potent and/or efficacious therapeutic intervention to treat lung fibrosis, e.g., idiopathic pulmonary fibrosis compared to the currently used anti-inflammatories and immunosuppressants or compared to small molecules that are only able to inhibit either, but not both of, Wnt- or TGFβ-mediated β-catenin signaling pathways. To date, the present inventors are the first to identify such small molecule inhibitors (see, e.g., FIGS. 1-114, and Table 1).

2. Liver Fibrosis

In various embodiments, the present invention contemplates, in part, a method of preventing, reversing, treating, and/or ameliorating liver fibrosis in a subject comprising administering one or more inhibitors of β-catenin signaling to the subject, wherein the inhibitor inhibits or reduces Wnt- and TGFβ-mediated β-catenin signaling.

A subject in need of treatment for liver fibrosis with small molecule inhibitors of the present invention includes subjects that have or are at risk of developing liver disease. In particular, the subject has, or is at risk of developing, liver fibrosis. The fibrosis can be at an early stage or may have progressed to a more advanced stage. In some cases, the fibrosis can have progressed to such a stage that the individual has liver cirrhosis. The subject can also display inflammation in regions of the liver and necrotic or degenerating cells can be present in the liver.

The subject to be treated can have an inherited disease that causes, or increases the risk of, liver disease and in particular of liver fibrosis, e.g., hepatic hemochromatosis, Wilson's disease, autoimmune disease, or alpha-1-antitrypsin deficiency. The subject to be treated can have liver disease due to a xenobiotic cause such as exposure to chemicals e.g., Rezulin®, Serzone® or other drugs thought to cause liver damage; chemicals in an industrial or agricultural context; plants containing pyrrolizidine alkaloid; and environmental toxins thought to cause liver fibrosis. Liver fibrosis may also be alcohol-induced. Thus, the subject to be treated can be, or could have been, an alcoholic.

In other embodiments, the subject may have one or more of a number of other conditions known to result in liver fibrosis such as, for example, primary biliary cirrhosis, autoimmune chronic active hepatitis, and/or schistosomiasis. The subject can have or could have had, a bile duct blockage. In some cases, the underlying cause of the fibrosis can be unknown. For example, the subject is one diagnosed as having cryptogenic cirrhosis.

Liver or hepatic fibrosis results from damage to the liver and is characterized by accumulation of extracellular matrix proteins (e.g., type I, II and/or III collagens, laminin, fibronectin and proteoglycans). Although the liver has some capacity for the breakdown of extracellular matrix, in some cases fibrosis is not resolved and progressively increases. Liver fibrosis may result in impairment of liver function with the fibrotic material disturbing the organization of the liver, altering blood flow and causing destruction of liver cells. Liver fibrosis may progress to cirrhosis, characterized by nodules of regenerating hepatocytes.

Causes of liver fibrosis include: increased EMT, increased deposition of extracellular matrix, pathogens (e.g., hepatitis B, C, or D virus), autoimmune conditions, exposure to a drug, exposure to a chemical, consumption of alcohol, inherited conditions, and primary biliary cirrhosis. Liver fibrosis associated with any of these diseases, or signs or clinical symptoms associated with liver fibrosis, can be treated using the methods described herein.

Liver fibrosis is the final common pathway for most chronic liver diseases (Brenner, Trans Am Clin Climatol Assoc. 2009; 120:361-8). Evidence supports a role for the activated myofibroblasts in hepatic fibrosis. The myofibroblast can be derived from epithelial to mesenchymal transition of liver epithelial cells. Increased TGF-β signaling contributes to the increased EMT in liver fibrosis (Ismail and Pinzani. Saudi J. Gastroenterol. 2009 January; 15(1):72-9; Zeisberg et al., J Biol Chem. 2007 Aug. 10; 282(32):23337-47). Similarly, increased Wnt signaling is associated with hepatic stellate cell activation and liver fibrosis. In fact, inhibition of Wnt signaling in hepatic stellate cells (HSGs; liver fibroblasts) reduced ECM deposition, β-catenin expression, and reduced liver fibrosis caused by diminished adipogenic transcription (Cheng et al., Am J Physiol Gastrointest Liver Physiol. 2008 January; 294(1):G39-49JH). Moreover, overexpression of β-catenin in rat livers can accelerate the development of liver cirrhosis compared to control rats (Hong et al., Anat Rec (Hoboken). 2009 June; 292(6):818-260)

However, currently, no acceptable therapeutic strategies exist, other than removal of the fibrogenic stimulus, to treat this potentially devastating liver fibroproliferative disease. Without wishing to be bound by any particular theory, the present invention contemplates, in part, that small molecules identified as inhibitors of both Wnt- and TGFβ-mediated β-catenin signaling provide a more potent and/or efficacious therapeutic intervention to treat liver fibrosis compared to small molecules that are only able to inhibit either, but not both of, Wnt- or TGFβ-mediated β-catenin signaling pathways.

3. Kidney Fibrosis

In various embodiments, the present invention contemplates, in part, a method of preventing, reversing, treating, and/or ameliorating kidney fibrosis in a subject comprising administering one or more inhibitors of β-catenin signaling to the subject, wherein the inhibitor inhibits or reduces Wnt- and TGFβ-mediated β-catenin signaling.

A subject in need of treatment for kidney fibrosis with small molecule inhibitors of Wnt- and TGFβ-mediated β-catenin signaling include subjects having or that are at risk of developing chronic renal failure (CRF), diabetic nephropathy, glomerulosclerosis, glomerular nephritis, nephritis associated with systemic lupus, cancer, physical obstructions, toxins, metabolic disease and immunological diseases, all of which may culminate in kidney fibrosis or fibroproliferative disease.

Kidney fibrosis results from damage to the kidney and is characterized by accumulation of extracellular matrix proteins and increased EMT in kidney tubular epithelial cells (Kalluri and Neilson. J. Clin. Invest. 2003; 112:1776-1784). Kidney epithelial cells may be particularly prone to EMTs that occur in response to inflammatory stress and lead to pathologic fibrosis (Aufderheide et al., J. Cell Biol. 1987; 105:599-608; Ivanova et al., Am. J. Physiol. Renal Physiol. 2008; 294:F1238-F12480). Several groups have established a role for TGF-β in contributing to various forms of kidney fibrosis through increased EMT and fibroblast activation (Zeisberg et al. Nat. Med. 2003; 9:964-968; Hills and Squires. Am J Nephrol. 2010; 31(1):68-74; August et al., Trans Am Clin Climatol Assoc. 2009; 120:61-72P; Chea and Lee, Yonsei Med J. 2009 Feb. 28; 50(1):105-11). TGF-β induces EMT via both a Smad2/3-dependent pathway and a MAPK-dependent pathway. Recent experiments have also demonstrated a key role for the E-cadherin/β-catenin signaling axis for EMT involving epithelial cells (Kim et al. Cell Biol. Int. 2002; 26:463-476; Nawshad et al. Cells Tissues Organs. 2005; 179:11-23). The Wnt pathway also plays a role in kidney fibrosis (Pulkkinen et al., Organogenesis. 2008 April; 4(2):55-9). Wnt-4 expression is induced four murine models of renal injury that produce tubulointerstitial fibrosis: folic acid-induced nephropathy, unilateral ureteral obstruction, renal needle puncture, and genetic polycystic kidney disease (Surendran and Simon, Am J Physiol Renal Physiol. 2003 April; 284(4):F653-62; Surendran et al., J Am Soc Nephrol. 2005 August; 16(8):2373-84; and Surendran et al., Am J Physiol Renal Physiol. 2002 March; 282(3):F431-41). Wnt-4 expression was induced in the collecting duct epithelium followed by myofibroblast activation and deposition of extracellular matrix (ECM) proteins, e.g., Col1a-1, fibronectin, in fibrotic lesions surrounding the collecting ducts (Surendran et al., 2005; Surendran et al., 2002).

Endothelial to mesenchymal transition (EnMT) is another cell signaling pathway that regulates kidney fibrosis. About 35% of fibroblasts in kidney fibrosis were derived via EnMT from the endothelial cells normally residing within the kidney (Kalluri and Neilson. J. Clin. Invest. 2003; 112:1776-1784). Another group published similar findings, in that EnMT contributed approximately 30 to 50% of pathological fibroblasts in three mouse models of chronic kidney disease: (1) Unilateral ureteral obstructive nephropathy, (2) streptozotocin-induced diabetic nephropathy, and (3) a model of Alport renal disease (Zeisberg et al., J Am Soc Nephrol. 2008 December; 19(12):2246-8EM). EnMT transition can also be regulated by TGF-β, Wnt, and β-catenin signaling pathways (Potenta et al., Br J. Cancer. 2008 Nov. 4; 99(9): 1375-1379).

However, currently, no acceptable therapeutic strategies exist to treat kidney fibrosis or fibroproliferative disease. Without wishing to be bound by any particular theory, the present invention contemplates, in part, that small molecules identified as inhibitors of both Wnt- and TGFβ-mediated β-catenin signaling provide a more potent and/or efficacious therapeutic intervention to treat kidney fibrosis compared to small molecules that are only able to inhibit either, but not both of, Wnt- or TGFβ-mediated β-catenin signaling pathways.

4. Systemic Sclerosis

In various embodiments, the present invention contemplates, in part, a method of preventing, reversing, treating, and/or ameliorating systemic sclerosis in a subject comprising administering one or more inhibitors of β-catenin signaling to the subject, wherein the inhibitor inhibits or reduces Wnt- and TGFβ-mediated β-catenin signaling.

Systemic sclerosis is a degenerative disorder in which excessive fibrosis occurs in multiple organ systems, including the skin, blood vessels, heart, lungs, and kidneys. Several forms of fibrotic diseases cause death in scleroderma patients, including pulmonary fibrosis, congestive heart failure, and renal fibrosis; each of which occurs in about half of systemic sclerosis patients. TGF-β contributes to fibroblast activation, collagen overproduction (ECM deposition), and increased EMT in pathological fibrosis associated with systemic sclerosis. Neutralizing antibodies that block TGF-β activation or function are effective in shutting down TGF-β signaling and selectively inhibit the progression of fibrosis associated with systemic sclerosis (Varga, Bull NYU Hosp Jt Dis. 2008; 66(3):198-202J). Without wishing to be bound by any particular theory, the present invention contemplates, in part, that because the forms of fibrosis associated with systemic sclerosis, e.g., lung fibrosis, kidney fibrosis, and liver fibrosis, share related pathways, small molecules identified as inhibitors of both Wnt- and TGFβ-mediated β-catenin signaling provide a more potent and/or efficacious therapeutic intervention to treat systemic sclerosis compared to small molecules that are only able to inhibit either, but not both of, Wnt- or TGFβ-mediated β-catenin signaling pathways.

The foregoing examples share increased EMT/EnMT, prolonged fibroblast activation, and increased deposition of ECM as mechanisms contributing to the progressive fibrosis or fibroproliferative disease. Previous studies demonstrate roles for each of these mechanisms in various forms of fibrosis, e.g., lung, liver, and kidney fibrosis, among others. To date, the present inventors are the first to identify small molecule inhibitors in a fibrosis model that shows increased EMT, prolonged fibroblast activation, and increased deposition of ECM (see, e.g., FIGS. 1-114, and Table 1).

The present invention contemplates that treatment of any type of fibrosis or fibroproliferative disease with one or more small molecule inhibitors of Wnt- and TGF-β-mediated β-catenin signaling would be effective to prevent, reverse, treat, or ameliorate fibrotic diseases in diverse tissues and organs that show increased EMT/EnMT, prolonged fibroblast activation, and increased deposition of ECM.

Accordingly, the present invention provides improved compositions and methods for treating fibrosis and fibroproliferative disease using small molecule inhibitors of Wnt- and TGF-β-mediated β-catenin signaling.

5. Transplant Rejections

In various embodiments, the present invention contemplates, in part, a method of preventing, reversing, treating, and/or ameliorating transplant rejection in a subject comprising administering one or more inhibitors of β-catenin signaling to the subject, wherein the inhibitor inhibits or reduces Wnt- and TGFβ-mediated β-catenin signaling. The transplant rejection can be rejection of a transplanted organ or tissue or tissue graft. Exemplary transplanted tissues include, but are not limited to bones, corneas, as well as major organs such as hearts, kidneys, livers, lungs, skin, and pancreases.

As used herein, the term “transplantation” refers to the process of taking a cell, tissue, or organ, called a “transplant” or “graft” from one subject and placing it into a (usually) different subject. The subject who provides the transplant is called the “donor” and the subject who received the transplant is called the “recipient.” An organ, or graft, transplanted between two genetically different subjects of the same species is called an “allograft”. A graft transplanted between subjects of different species is called a “xenograft”. As used herein, the term “transplant rejection” is defined as functional and structural deterioration of the organ due to an active immune response expressed by the transplant recipient, and independent of non-immunologic causes of organ dysfunction.

As used herein, the term “acute rejection” (e.g., of a transplant) refers to a rejection of a transplanted organ developing after the first 5-60 post-transplant days. It is generally a manifestation of cell-mediated immune injury. It is believed that both delayed hypersensitivity and cytotoxicity mechanisms are involved. The immune injury is directed against HLA, and possibly other cell-specific antigens expressed by the tubular epithelium and vascular endothelium. As used herein, the term “chronic rejection” (e.g., of a transplant) represents a consequence of combined immunological injury (e.g., chronic rejection) and non-immunological damage (e.g., hypertensive nephrosclerosis, or nephrotoxicity of immuno-suppressants like cyclosporine A), occurring months or years after transplantation and ultimately leading to fibrosis and sclerosis of the allograft, associated with progressive loss of organ function.

Without wishing to be bound by any particular theory, the present invention contemplates, in part, fibrosis involved in tissue and/or organ rejection is mediated by Wnt and TGF-β signaling through β-catenin and that small molecules identified as inhibitors of both Wnt- and TGFβ-mediated β-catenin signaling provide a more potent and/or efficacious therapeutic intervention to treat fibrosis arising from tissue or organ rejection compared to small molecules that are only able to inhibit either, but not both of, Wnt- or TGFβ-mediated β-catenin signaling pathways.

### D. Small Molecules

The present invention also provides, in part, compositions and methods directed to the use of small molecule inhibitors of Wnt- and TGF-β-mediated β-catenin signaling to reduce fibrosis or ameliorate and/or prevent fibroproliferative disease. As used herein, the terms “small molecule,” “compound,” are used interchangeably herein, with the interchangeable terms “test compound,” and “candidate compound” referring to compounds tested in the cell-based assays. In particular embodiments, small molecules of the invention inhibit or antagonize Wnt- and TGF-β-mediated β-catenin signaling, and thus, the terms “small molecule inhibitor” and “antagonist” refer to those compounds having a desired activity in modulating, inhibiting, down-regulating, reducing, ameliorating, preventing, or blocking fibrosis or a fibroproliferative disease. Small molecule inhibitors of the invention may inhibit the level of an indicator of Wnt- and TGF-β-mediated β-catenin signaling in a cell about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or may completely inhibit an indicator of Wnt- and TGF-β-mediated β-catenin signaling in a cell compared to an cell that has not been exposed to the inhibitor.

In one embodiment, the term “small molecule” refers to numerous biological classes, including synthetic, semi-synthetic, or naturally-occurring inorganic or organic molecules, including synthetic, recombinant or naturally-occurring compounds. In a particular embodiment, the term “small molecule” refers to chemical classes, including synthetic, semi-synthetic, or naturally-occurring inorganic or organic molecules, including synthetic, recombinant or naturally-occurring compounds. “Test compounds” include those found in large libraries of synthetic or natural compounds. One having ordinary skill in the art would appreciate that assays of the present invention are suitable for determining inhibitory activity of a small molecule in a Wnt- and TGF-β-mediated β-catenin signaling assay, as described elsewhere herein.

In particular embodiments, small molecule inhibitors of Wnt- and TGF-β-mediated β-catenin signaling are obtained from a combinatorial small organic molecule or peptide library containing a large number of potential therapeutic compounds (e.g., anti-fibrotic compounds). Such “combinatorial chemical libraries” or “ligand libraries” can be screened separately or screened in pools, to identify those library members, particular chemical species or subclasses that display the desired characteristic activity of inhibiting Wnt- and TGF-β-mediated β-catenin signaling. In certain embodiments, screening libraries with pools of compounds may reduce the ultimate number of screens for any given library. For example, pools containing the activity of interest can be iterively subdivided until the activity is restricted to a particular compound or mixture of compounds. The identified compounds can serve as conventional “lead compounds” or can themselves be used as potential or actual therapeutics to prevent and/or treat fibrosis or fibroproliferative disease.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks in every possible way for a given compound length. Millions of chemical compounds can be synthesized through such combinatorial mixing of chemical building blocks. Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 1991; 37:487-493 and Houghton et al., Nature. 1991; 354:84-88). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA. 1993; 90:6909-6913), vinylogous polypeptides (Hagihara et al., J. Amer. Chem. Soc. 1992; 114:6568), nonpeptidal peptidomimetics with glucose scaffolding (Hirschmann et al., J. Amer. Chem. Soc. 1992; 114:9217-9218), analogous organic syntheses of small compound libraries (Chen et al., J. Amer. Chem. Soc. 1994; 116:2661), oligocarbamates (Cho et al., Science. 1993; 261:1303), and/or peptidyl phosphonates (Campbell et al., J. Org. Chem. 1994; 59:658), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science. 1996; 274:1520-1522 and U.S. Pat. No. 5,593,853), small organic molecule libraries (see, e.g., benzodiazepines, Baum, C&EN. 1993 Jan. 18, page 33; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514. Additional illustrative examples for the synthesis of molecular libraries can be found in: (Carell et al., 1994a; Carell et al., 1994b; Cho et al., 1993; DeWitt et al., 1993; Gallop et al., 1994; Zuckermann et al., 1994). In addition, libraries of natural compounds in the form of bacterial, fungal, plant and animal extracts are available from Pan Labs (Bothell, Wash.) or are readily producible.

In particular embodiments, small molecules of the present invention include small organic or inorganic compounds having a molecular weight of more than 50 and less than about 2,500 Daltons. Small molecules may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Agents, particularly candidate agents, are also found among biomolecules including peptides, saccharides, fatty acids, steroids, purines, pyrimidines, derivatives, structural analogs or combinations thereof.

In other embodiments, the small molecule can be purified or can be contained in a complex substance. A complex substance is comprised of a plurality of components and/or compounds, including one or more small molecules. A complex substance can, for example, be an animal's body fluid. Suitable animal body fluids include, for example, blood, plasma, serum, bone marrow, urine, cerebrospinal fluid, saliva, synovial fluid, ocular fluid, amniotic fluid, bile, seminal fluid, or secretions. Suitable secretions include pancreatic secretions, gastric secretions, nasal secretions, pulmonary secretions, vaginal secretions, and perspiration. Accordingly, the substances identified herein are in no way limiting. The animal providing the small molecule can be a human patient. Furthermore, there is no need in the context of the invention to identify the nature or any characteristics of the small molecule. Accordingly, the invention encompasses embodiments in which the nature and characteristics of the test compound is unknown, yet the function of which, inhibition of Wnt- and TGF-β-mediated β-catenin signaling can readily be determined using the small molecule screening assays, described elsewhere herein.

Exemplary small molecule inhibitors of Wnt- and TGF-β-mediated β-catenin signaling suitable for use in the compositions and methods of the present invention include, but are not limited to, FT-1055-3, FT-1067-3, FT-1069-1, FT-1083-1, FT-1147-3, FT-1150-3, FT-1202-1, FT-1203-1, FT-1812-4, FT-1265-1, FT-1281-1, FT-1294-5, FT-1301-1, FT-1320-1, FT-1355-2, FT-1361-2, FT-1366-2, FT-1398-2, FT-1434-2, FT-1435-2, FT-1436-1, FT-1480-1, FT-1497-1, FT-1504-3, FT-1515-1, FT-1517-1, FT-1518-1, FT-1532-1, FT-1575-2, FT-1609-1, FT-1612-3, FT-1613-1, FT-1660-1, FT-1678-1, FT-1688-1, FT-1693-1, FT-1812-3, FT-1915-2, FT-1986-3, FT-1992-3, FT-2014-2, FT-2046-2, FT-2051-2, FT-2081-2, FT-2103-2, FT-2115-2, FT-2228-3, FT-2254-2, FT-2318-2, FT-2342-2, FT-2474-2, FT-2498-2, FT-2562-3, FT-2580-2, FT-2619-2, FT-2633-2, FT-2660-2, FT-2691-2, FT-2693-3, FT-2770-2, FT-2820-2, FT-2862-2, FT-2863-2, FT-2907-2, FT-2909-2, FT-2912-3, FT-2920-3, FT-2947-2, FT-2948-2, FT-2968-2, FT-2974-2, FT-3027-2, FT-3052-2, FT-3062-2, FT-3073-2, FT-3093-2, FT-3128-2, FT-3197-2, FT-3216-2, FT-3352-2, FT-3386-2, FT-3422-2, FT-3489-2, FT-3512-2, FT-3515-2, FT-3548-2, FT-3564-2, FT-3687-2, FT-3703-2, FT-3801-2, FT-3852-2, FT-3872-2, FT-3873-2, FT-3881-2, FT-3883-2, FT-3886-2, FT-3893-2, FT-3897-2, FT-3907-2, FT-3908-2, FT-3934-2, FT-3935-2, FT-3937-2, FT-3938-2, FT-3941-2, FT-3951-2, FT-3954-2, FT-3959-2, FT-3963-2, FT-3967-2, FT-3985-2, FT-3999-2, FT-4001-1, and FT-4145-2 (see, e.g., Examples 1 and 2; FIGS. 1-114; and Table 1).

In particular embodiments, small molecule inhibitors of Wnt- and TGF-β-mediated β-catenin signaling are selected from the group consisting of: FT-1067, FT-2907, FT-3934, FT-3938, FT-3951, FT-3967, and FT-4001.

### E. Screening Assays

In various embodiments, the present invention provides a method of identifying a test compound as a small molecule inhibitor of Wnt- and TGF-β-mediated β-catenin signaling. In one embodiment, a method for identifying an inhibitor of Wnt- and TGF-β-mediated β-catenin signaling comprises determining the ability of the test compound to inhibit both Wnt- and TGF-β-mediated β-catenin signaling in a cell compared to a cell lacking the test compound.

In a particular embodiment, a method for identifying an inhibitor of Wnt- and TGF-β-mediated β-catenin signaling comprises stimulating Wnt-mediated β-catenin signaling in a cell and measuring the level of Wnt-mediated β-catenin signaling in the presence and absence of a test compound. For example, if a test compound decreases the level of Wnt-mediated β-catenin signaling in a cell compared to the level of Wnt-mediated β-catenin signaling in an untreated control cell, the test compound can be classified as an inhibitor of Wnt-mediated β-catenin signaling.

Exemplary methods of stimulating Wnt-mediated β-catenin signaling include, without limitation, contacting a cell with a WNT ligand (e.g., WNT3A, WNT1). Other exemplary WNT ligands include Wnt2, Wnt2b/13, Wnt3, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16.

Wnt-mediated β-catenin signaling can also be stimulated by culturing a cell in WNT conditioned media, e.g., WNT3A conditioned medium, or by contacting a cell with a small molecule inhibitor of glycogen synthase kinase 3β (GSK 3B), e.g., BIO. Other methods and compounds for stimulating Wnt-mediated β-catenin signaling are known in the art and may, in particular embodiments, be applied to the screening methods of the present invention.

The method for identifying an inhibitor of Wnt- and TGF-β-mediated β-catenin signaling also comprises stimulating TGF-β-mediated β-catenin signaling in a cell and measuring the level of TGF-β-mediated β-catenin signaling in the presence and absence of a test compound. For example, if a test compound decreases the level of TGF-β-mediated β-catenin signaling in a cell compared to the level of TGF-β-mediated β-catenin signaling in an untreated control cell, the test compound can be classified as an inhibitor of TGF-β-mediated β-catenin signaling.

Exemplary methods of stimulating TGF-β-mediated β-catenin signaling include, without limitation, contacting a cell with a TGF-b ligand (e.g., TGF-β1, TGF-β2, TGF-β3). TGF-β-mediated β-catenin signaling can also be stimulated by methods and compounds known in the art, which may, in particular embodiments, be applied to the screening methods of the present invention.

In preferred embodiments, the small molecule compounds of the invention can inhibit both Wnt- and TGF-β-mediated β-catenin signaling.

In one embodiment, a method for identifying an inhibitor of Wnt- and TGF-β-mediated β-catenin signaling comprises i) activating or stimulating Wnt-mediated β-catenin signaling in a first population of cells and measuring the level of an indicator of Wnt-mediated β-catenin signaling in the presence and absence of a test compound; ii) activating TGF-β-mediated β-catenin signaling in a second population of cells and measuring the level of an indicator of TGFβ-mediated β-catenin signaling in the presence and absence of the test compound; iii) comparing the levels of the indicators of β-catenin signaling measured in step i) and step ii) in the presence and absence of the test compound; and identifying the test compound as a Wnt- and TGF-β-mediated β-catenin signaling by observing a decrease, reduction, and/or inhibition in the levels of the indicator of Wnt-mediated β-catenin signaling and the indicator of TGF-β-mediated β-catenin signaling in the populations of first and second cells in the presence of the test compound compared to levels of the indicator of Wnt-mediated β-catenin signaling and the indicator of TGF-β-mediated β-catenin signaling in populations of first and second cells in the absence of the test compound.

As used herein, the term “indicator of Wnt-mediated β-catenin signaling” refers to a reporter gene construct comprising a β-catenin responsive promoter (e.g., has LEF/TCF transcription factor binding sites) that, when activated, increases the expression of a reporter gene, e.g., firefly luciferase, GFP, and the like. Wnt-mediated β-catenin signaling reporter constructs are known in the art and commercially available. As used herein, the term “indicator of TGF-β-mediated β-catenin signaling” refers to a reporter gene construct comprising a β-catenin responsive promoter (e.g., has LEF/TCF transcription factor binding sites) that, when activated, increases the expression of a reporter gene, e.g., firefly luciferase, GFP, and the like. In preferred embodiments, the indicator of Wnt-mediated β-catenin signaling is identical to the indicator of indicator of TGF-β-mediated β-catenin signaling. In other embodiments, the indicators are not identical.

In other certain embodiments, the indicator used in the assays may indicate whether the population of cells contacted with a test compound have undergone an EMT or EnMT (both mediated by β-catenin signaling pathways) in response to being contacted with the compound. In general, such an indicator can be an epithelial, endothelial, or mesenchymal cell surface marker. In one-non-limiting example, if a test compound increases EMT in a population of cells, the population of cells will have increased expression of mesenchymal cell markers and/or a decreased expression of epithelial cell markers compared to a population of cells that has not been contacted with the compound. In another, non-limiting example, if a test compound increases EnMT in a population of cells, the population of cells will have increased expression of mesenchymal cell markers and/or a decreased expression of endothelial cell markers compared to a population of cells that has not been contacted with the compound.

Illustrative examples of epithelial markers that can be used in any of the methods of this invention include phospho-14-3-3 epsilon, 14-3-3 gamma (KCIP-I), 14-3-3 sigma (Stratifin), 14-3-3 zeta/delta, phospho-serine/threonine phosphatase 2 A, 4F2hc(CD98 antigen), adenine nucleotide translocator 2, annexin A3, ATP synthase β chain, phospho-insulin receptor substrate p53/p54, Basigin (CD 147 antigen), phospho-CRK-associated substrate (pl30Cas), BcI-X, phospho-P-cadherin, phospho-calmodulin (CaM), Calpain-2 catalytic subunit, Cathepsin D, Cofilin-1, Calpain small subunit 1, Catenin β-1, Catenin delta-1 (pi 20 catenin), Cystatin B, phospho-DAZ-associated protein 1, Carbonyl reductase [NADPH], Diaphanous-related formin 1 (DRFI), Desmoglein-2, Elongation factor 1-delta, phospho-pl85erbB2, Ezrin (p81), phospho-focal adhesion kinase 1, phospho-p94-FER (c-FER), Filamin B, phospho-GRB2-associated binding protein 1, Rho-GDI alpha, phospho-GRB2, GRP 78, Glutathione S-transferase P, 3-hydroxyacyl-CoA dehydrogenase, HSP 90-alpha, HSP70.1, eIF3 pi 10, eIF-4E, Leukocyte elastase inhibitor, Importin-4, Integrin alpha-6, Integrin β-4, phospho-Cytokeratin 17, Cytokeratin 19, Cytokeratin 7, Casein kinase I, alpha, Protein kinase C, delta, Pyruvate kinase, isozymes M1/M2, phospho-Erbin, LIM and SH3 domain protein 1 (LASP-I), 4F21c (CD98 light chain), L-lactate dehydrogenase A chain, Galectin-3, Galectin-3 binding protein, phospho-LIN-7 homolog C, MAP (APC-binding protein EBI), Maspin precursor (Protease inhibitor 5), phospho-Met tyrosine kinase (HGF receptor), Mixed-lineage leukemia protein 2, Monocarboxylate transporter 4, phospho-C-Myc binding protein (AMY-I), Myosin-9, Myosin light polypeptide 6, Nicotinamide phosphoribosyltransferase, Niban-like protein (Meg-3), Ornithine aminotransferase, phospho-Occludin, Ubiquitin thiolesterase, PAF acetylhydrolase IBR subunit, phospho-partitioning-defective 3 (PAR-3), phospho-programmed cell death 6-interacting protein, phospho-Programmed cell death protein 6, Protein disulfide-isomerase, phospho-plakophilin-2, phospho-plakophilin-3, Protein phosphatase 1, Peroxiredoxin 5, Proteasome activator complex subunit 1, Prothymosin alpha, Retinoic acid-induced protein 3, phospho-DNA repair protein REVI, Ribonuclease inhibitor, RuvB-like 1, S-100P, S-100L, Calcyclin, SIOOC, phospho-Sec23A, phospho-Sec23B, Lysosome membrane protein II (LIMP II), p60-Src, phospho-Amplaxin (EMSI), SLP-2, Gamma-synuclein, Tumor calcium signal transducer 1, Tumor calcium signal transducer 2, Transgelin-2, Transaldolase, Tubulin (3-2 chain, Translationally controlled (TCTP), Tissue transglutaminase, Transmembrane protein Tmp21, Ubiquitin-conjugating enzyme E2 N, UDP-glucosyltransferase 1, phospho-p61-Yes, phospho-Tight junction protein ZO-1, AHNAK (Desmoyokin), phospho-ATP synthase β chain, phospho-ATP synthase delta, Cold shock domain protein El, Desmoplakin III, Plectin 1, phospho-Nectin 2 (CDI 12 antigen), phospho-pl85-Ron, phospho-SHCI, E-cadherin, Brk, γ-catenin, αl-catenin, α2-catenin, α3-catenin, keratin 8, keratin 18, connexin 31, plakophilin 3, stratafin 1, laminin alpha-5, ST14, and other epithelial biomarkers known in the art (see for example, US Patent Application Publication 2007/0212738; U.S. Patent Application 60/923,463; U.S. Patent Application 60/997,514).

Illustrative examples of endothelial cell markers suitable for use with the present invention are: 7B4 antigen, ACE, BNH9/BNF13, CD31 (PECAM-1), CD31, CD34, CD54 (ICAM-1), CD62P (p-Selectin GMP140), CD105 (Endoglin), CD146 (P1H12), D2-40, E-selectin, EN4, Endocan, ESM-1, Endoglin (CD105), Endoglyx-1, Endomucin, Endosialin (tumor endothelial marker 1, TEM-1, FB5), Eotaxin-3, EPAS1 (Endothelial PAS domain protein 1), Factor VIII related antigen, FB21, Flk-1 (VEGFR-2), Flt-1 (VEGFR-1), GBP-1 (guanylate-binding protein-1), GRO-alpha, Hex, ICAM-2 (intercellular adhesion molecule 2), LYVE-1, MECA-32, MECA-79, MRB (magic roundabout), Nucleolin, PAL-E (pathologische anatomie Leiden-endothelium), RPTPmu (Receptor protein tyrosine phosphatase mu), sVCAM-1, TEM1 (Tumor endothelial marker 1), TEM5 (Tumor endothelial marker 5), TEM7 (Tumor endothelial marker 7), TEM8 (Tumor endothelial marker 8), Thrombomodulin (TM, CD141), Tie-2, VCAM-1 (vascular cell adhesion molecule-1) (CD106), VE-cadherin (CD144), vWF (von Willebrand factor), and the like.

Illustrative examples of additional mesenchymal markers that can be used in any of the methods of this invention include MMP9 (matrix-metalloproteinase 9; NCBI Gene ID No. 4318), MHC class I antigen A*l, Acyl-CoA desaturase, LANP-like protein (LANP-L), Annexin A6, ATP synthase gamma chain, BAG-family molecular chaperone regulator-2, phospho-Bullous pemphigoid antigen, phospho-Protein Clorf77, CDKI (cdc2), phospho-Clathrin heavy chain 1, Condensin complex subunit 1, 3,2-trans-enoyl-CoA isomerase, DEAH-box protein 9, phospho-Enhancer of rudimentary homolog, phospho-Fibrillarin, GAPDH muscle, GAPDH liver, Synaptic glycoprotein SC2, phospho-Histone H 1.0, phospho-Histone H 1.2, phospho-Histone HI 0.3, phospho-Histone HI 0.4, phospho-Histone HI 0.5, phospho-Histone HIx, phospho-Histone H2AFX, phospho-Histone H2A.0, phospho-Histone H2A.q, phospho-Histone H2A.z, phospho-Histone H2B.j, phospho-Histone H2B.r, phospho-Histone H4, phospho-HMG-17-like 3, phospho-HMG-14, phospho-HMG-17, phospho-HMGI-C, phospho-HMG-I/HMG-Y, phospho-Thyroid receptor interacting protein 7 (TRIP7), phospho-hnRNP H3, hnRNP C1/C2, hnRNP F, phospho-hnRNP G, eIF-5A, NFAT 45 kDa, Importin β-3, cAMP-dependent PKIa, Lamin BI, Lamin A/C, phospho-Laminin alpha-3 chain, L-lactate dehydrogenase B chain, Galectin-1, phospho-Fezl, Hyaluronan-binding protein 1, phospho-Microtubule-actin crosslinking factor 1, Melanoma-associated antigen 4, Matrin-3, Phosphate carrier protein, Myosin-10, phospho-N-acylneuraminate cytidylyltransferase, phospho-NHP2-like protein 1, H/AC A ribonucleoprotein subunit 1, Nucleolar phosphoprotein pi 30, phospho-RNA-binding protein Nova-2, Nucleophosmin (NPM), NADH-ubiquinone oxidoreductase 39 kDa subunit, phospho-Polyadenylate-binding protein 2, Prohibitin, Prohibitin-2, Splicing factor Prp8, Polypyrimidine tract-binding protein 1, Parathymosin, Rab-2A, phospho-RNA-binding protein Raly, Putative RNA-binding protein 3, phospho-60S ribosomal protein L23, hnRNP AO, hnRNP A2/B1, hnRNP A/B, U2 small nuclear ribonucleoprotein B, phospho-Ryanodine receptor 3, phospho-Splicing factor 3A subunit 2, snRNP core protein D3, Nesprin-1, Tyrosine-tRNA ligase, phospho-Tankyrase 1-BP, Tubulin β-3, Acetyl-CoA acetyltransferase, phospho-bZIP enhancing factor BEF (Aly/REF; Tho4), Ubiquitin, Ubiquitin carboxyl-terminal hydrolase 5, Ubiquinol-cytochrome c reductase, Vacuolar protein sorting 16, phospho-Zinc finger protein 64, phospho-AHNAK (Desmoyokin), ATP synthase β chain, ATP synthase delta chain, phospho-Cold shock domain protein El, phospho-Plectin 1, Nectin 2 (CDI 12 antigen), pl85-Ron, SHCI, vimentin, fibronectin, fibrillin-1, fibrillin-2, collagen alpha-2(IV), collagen alpha-2(V), LOXLI, nidogen, CI lorf9, tenascin, N-cadherin, embryonal EDB+ fibronectin, tubulin alpha-3, epimorphin, and other mesenchymal biomarkers known in the art, (see for example, US Patent Application Publication 2007/0212738; U.S. Patent Application 60/923,463; U.S. Patent Application 60/997,514).

Suitable populations of cells used in methods of identifying a small molecule inhibitor of Wnt- and TGF-β-mediated β-catenin signaling comprise epithelial or endothelial cells of the lung, liver, kidney, gut, eye, or heart. The cells may be cell lines, e.g., A549 cells, or be primary populations of cells, e.g., ATII cells. The screening assays can be tailored to any cell type that develops fibrosis or that may be susceptible to fibrotic disease.

In particular embodiments, methods of the present invention comprise determining a dose response curve and/or an IC₅₀ of a test compound. As used herein, the term “dose response curve” describes a relationship between the amount of a compound assayed and the resulting measured response. The term “dose” is commonly used to indicate the amount of the compound used in the experiment, while the term “response” refers to the measurable effect of the compound tested. Dose-response relationships are determined graphically by plotting the varying compound concentration on the X-axis in log scale and the measurable response on the Y-axis. As used herein, the term “IC₅₀” means the concentration of a compound that is required to inhibit an indicator of Wnt- and TGF-β-mediated β-catenin signaling halfway between the baseline response and the maximum response of the indicator to that compound.

Generally, a dose-response curve for the test compound is determined by measuring the level of an indicator of Wnt- and TGF-β-mediated β-catenin signaling using various concentrations of a test compound, such as a set of serial dilutions of the test compound. The goal of determining the inhibitory activity of a small molecule inhibitor of Wnt- and TGF-β-mediated β-catenin signaling over a serially diluted concentration range is to provide for the construction of a dose response curve. The X-axis of a dose response curve generally represents the concentration of the test compound on a log scale, whereas the Y-axis represents the response of the in vitro indicator of Wnt- and TGF-β-mediated β-catenin signaling in response to a particular concentration of test compound.

Test compounds can be assayed at several concentrations within the range of about 1 nanomolar to about 100 millimolar. It will be possible or even desirable to conduct certain of these assays at concentrations of about 1 nanomolar to about 1 millimolar, 1 nanomolar to about 100 micromolar, or about 1 nanomolar to about 10 micromolar. In a particular embodiment, the range of concentrations to be tested consists of a plurality of 2, 3, 4, 5, 6, 7, 8, 9, or 10 or more different concentrations within a range of about 1 nanomolar to about 1 millimolar, or alternatively, within a concentration range of about 1 nanomolar to about 50 micromolar.

In a more particular embodiment, the plurality of different test compounds are 2-fold serial dilutions, 3-fold serial dilutions, 4-fold serial dilutions, 5-fold serial dilutions, 6-fold serial dilutions, 7-fold serial dilutions, 8-fold serial dilutions, 9-fold serial dilutions, or 10-fold serial dilutions in a range of concentrations of about 1 nanomolar to about 1 millimolar, or alternatively, within a concentration range of about 1 nanomolar to about 50 micromolar.

In certain embodiments, a high-throughput method for determining a small molecule inhibitor of Wnt- and TGF-β-mediated β-catenin signaling is contemplated. In such embodiments, the populations of cells are cultured in a tissue culture device having a plurality of wells, wherein the plurality of wells is selected from the group consisting of 4, 6, 12, 24, 48, 96, 384, and 1536 wells. In another embodiment, the tissue culture device is a microtiter plate having a plurality of wells. In a particular embodiment, the microtiter plate may have 4, 6, 12, 24, 48, 96, 384, or 1536 wells. In a more particular embodiment, the microtiter plate may have 24, 48, 96, or 384 wells. In a preferred embodiment, the microtiter plate has 48, 96, or 384 wells.

### F. Compositions

Compositions (i.e., medicaments) of the present invention include, but are not limited to pharmaceutical compositions. In particular embodiments, compositions (i.e., medicaments) of the present invention comprise one or more small molecule inhibitors of Wnt- and TGF-β-mediated β-catenin signaling, as described elsewhere herein, formulated with a pharmaceutically-acceptable salt for administration to a cell or an animal, either alone, or in combination with one or more other modalities of therapy. In particular embodiments, a composition comprises 1, 2, 3, 4, 5, or more small molecule inhibitors of Wnt- and TGF-β-mediated β-catenin signaling. In other particular embodiments, a composition comprises at least 1, at least 2, at least 3, at least 4, at least 5, or more small molecule inhibitors of Wnt- and TGF-β-mediated β-catenin signaling. A plurality of small molecule inhibitors of Wnt- and TGF-β-mediated β-catenin signaling (e.g., 1-5 inhibitors) can be combined in any number and any individual concentration.

Exemplary compositions of the present invention comprise one or more small molecule inhibitors of Wnt- and TGF-β-mediated β-catenin signaling selected from the group consisting of: FT-1055-3, FT-1067-3, FT-1069-1, FT-1083-1, FT-1147-3, FT-1150-3, FT-1202-1, FT-1203-1, FT-1812-4, FT-1265-1, FT-1281-1, FT-1294-5, FT-1301-1, FT-1320-1, FT-1355-2, FT-1361-2, FT-1366-2, FT-1398-2, FT-1434-2, FT-1435-2, FT-1436-1, FT-1480-1, FT-1497-1, FT-1504-3, FT-1515-1, FT-1517-1, FT-1518-1, FT-1532-1, FT-1575-2, FT-1609-1, FT-1612-3, FT-1613-1, FT-1660-1, FT-1678-1, FT-1688-1, FT-1693-1, FT-1812-3, FT-1915-2, FT-1986-3, FT-1992-3, FT-2014-2, FT-2046-2, FT-2051-2, FT-2081-2, FT-2103-2, FT-2115-2, FT-2228-3, FT-2254-2, FT-2318-2, FT-2342-2, FT-2474-2, FT-2498-2, FT-2562-3, FT-2580-2, FT-2619-2, FT-2633-2, FT-2660-2, FT-2691-2, FT-2693-3, FT-2770-2, FT-2820-2, FT-2862-2, FT-2863-2, FT-2907-2, FT-2909-2, FT-2912-3, FT-2920-3, FT-2947-2, FT-2948-2, FT-2968-2, FT-2974-2, FT-3027-2, FT-3052-2, FT-3062-2, FT-3073-2, FT-3093-2, FT-3128-2, FT-3197-2, FT-3216-2, FT-3352-2, FT-3386-2, FT-3422-2, FT-3489-2, FT-3512-2, FT-3515-2, FT-3548-2, FT-3564-2, FT-3687-2, FT-3703-2, FT-3801-2, FT-3852-2, FT-3872-2, FT-3873-2, FT-3881-2, FT-3883-2, FT-3886-2, FT-3893-2, FT-3897-2, FT-3907-2, FT-3908-2, FT-3934-2, FT-3935-2, FT-3937-2, FT-3938-2, FT-3941-2, FT-3951-2, FT-3954-2, FT-3959-2, FT-3963-2, FT-3967-2, FT-3985-2, FT-3999-2, FT-4001-1, and FT-4145-2.

In one particular illustrative embodiment, a composition comprises one or more small molecule inhibitors of Wnt- and TGF-β-mediated β-catenin signaling selected from the group consisting of: FT-1067, FT-2907, FT-3934, FT-3938, FT-3951, FT-3967, and FT-4001.

As described in detail below, compositions of the present invention comprising a combination of one or more small molecule inhibitors of Wnt- and TGF-β-mediated β-catenin signaling and a pharmaceutically acceptable cell salt, can be specially formulated for administration to a subject in need of treatment in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous, intraarterial, intravascular, or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally, as an inhalent or aerosol.

An “effective amount” refers to an amount of a small molecule inhibitor of Wnt- and TGF-β-mediated β-catenin signaling that is effective at dosages and for periods of time necessary, to achieve the desired therapeutic or prophylactic result. Effective amounts include therapeutically effective amounts and prophylactically effective (preventative) amounts. An effective amount may vary according to factors such as the disease state, age, sex, and weight of the individual, and the ability of the one or more repressors and/or activators to elicit a desired response in the individual.

A “therapeutically effective amount” of one or more small molecule inhibitors of Wnt- and TGF-β-mediated β-catenin signaling, as disclosed elsewhere is also one in which any toxic or detrimental effects of the small molecule inhibitor of Wnt- and TGF-β-mediated β-catenin signaling are outweighed by the therapeutically beneficial effects. The term “therapeutically effective amount” includes an amount that is effective to reduce, inhibit, prevent, or treat fibrosis or a fibroproliferative disease in a mammal (e.g., a subject in need of treatment). For example, a therapeutically effective amount of a small molecule inhibitor of Wnt- and TGF-β-mediated β-catenin signaling, can be an amount sufficient to cause a 5%, 10%, 15%, 20%, 25%, 30%, 35%, 40%, 45%, 50%, 55%, 60%, 65%, 70%, 75%, 80%, 90%, 95%, or 100% improvement in organ function (e.g., liver function, lung function, kidney function) relative to organ function observed prior to administration of the small molecule inhibitor of Wnt- and TGF-β-mediated β-catenin signaling.

A “prophylactically effective amount” refers to an amount of small molecule inhibitor of Wnt- and TGF-β-mediated β-catenin signaling that is effective at dosages and for periods of time necessary, to achieve the desired prophylactic result. Typically, but not necessarily, since a prophylactic dose is used in subjects prior to or at an earlier stage of disease, the prophylactically effective amount is less than the therapeutically effective amount.

As used herein, the term, “pharmaceutically-acceptable carrier” refers to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not adversely affecting the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) a pharmaceutically acceptable cell culture medium; and (23) other non-toxic compatible substances employed in pharmaceutical formulations.

Certain embodiments include “pharmaceutically-acceptable salts,” including hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., J. Pharm. Sci. 1977; 66:1-19). Additional examples include base addition salts such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

In another embodiment, the amount of active ingredient (e.g., small molecule inhibitor of Wnt- and TGF-β-mediated β-catenin signaling) in a single dosage from that is required to produce a therapeutic effect is about 0.1% active ingredient, about 1% active ingredient, about 5% active ingredient, about 10% active ingredient, about 15% active ingredient, about 20% active ingredient, about 25% active ingredient, about 30% active ingredient, about 35% active ingredient, about 40% active ingredient, about 45% active ingredient, about 50% active ingredient, about 55% active ingredient, about 60% active ingredient, about 65% active ingredient, about 70% active ingredient, about 75% active ingredient, about 80% active ingredient, about 85% active ingredient, about 90% active ingredient, or about 95% active ingredient or more, including all ranges of such values.

In certain embodiments, a composition of the present invention comprises an excipient selected from the group consisting of cyclodextrins and derivatives, celluloses, liposomes, micelle forming agents, e.g., bile acids, and polymeric carriers, e.g., polyesters and polyanhydrides; and a compound of the present invention. In certain embodiments, an aforementioned composition renders orally bioavailable one or more small molecule inhibitors of the present invention.

For administration by inhalation, a small molecule inhibitor of Wnt- and TGF-β-mediated β-catenin signaling for use according to the present invention can be conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch. In preferred embodiments, wherein the subject being treated has or is at risk of having fibrosis of the lung or a fibroproliferative disorder such as idiopathic pulmonary fibrosis, administration by inhalation can promote more effective delivery of a small molecule inhibitor of Wnt- and TGF-β-mediated β-catenin signaling to ATII lung epithelial cells and myofibroblasts of the lung.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A composition of the present invention may also be administered as a bolus, electuary or paste.

In solid dosage forms, for compositions of the invention suitable for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a composition as provided herein include powders, sprays, ointments, pastes, creams, lotions, gels, solutions, patches and inhalants. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a composition of the present invention to the body. Absorption enhancers can also be used to increase the flux of the agent across the skin.

Ophthalmic formulations, eye ointments, powders, solutions and the like, are also contemplated as being within the scope of this invention.

Compositions of this invention suitable for parenteral administration comprise pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Injectable depot forms are made by forming microencapsule matrices of the subject compounds in biodegradable polymers such as polylactide-polyglycolide. Examples of other biodegradable polymers include poly-(orthoesters) and poly-(anhydrides).

In certain embodiments, microemulsification technology may be utilized to improve bioavailability of lipophilic (water insoluble) small molecule inhibitors of Wnt- and TGF-β-mediated β-catenin signaling (Dordunoo et al., Drug Development and Industrial Pharmacy. 1991; 17(12), 1685-1713 and REV 5901 (Sheen, P. C, et al., J Pharm Sci 80(7), 712-714, 1991).

As used herein, the phrases “parenteral administration” and “administered parenterally” refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

As used herein, the terms “systemic administration,” “administered systemically,” “peripheral administration” and “administered peripherally” refer to the administration of a compound, drug or other material other than directly into the central nervous system, such that it enters the patient's system and, thus, is subject to metabolism and other like processes, for example, subcutaneous administration.

In general, a suitable daily dose of a composition comprising one or more small molecule inhibitors of Wnt- and TGF-β-mediated β-catenin signaling as described herein, will be that amount of the small molecule inhibitor which is the lowest dose effective to produce a therapeutic effect. Administration of one or more small molecule inhibitors of Wnt- and TGF-β-mediated β-catenin signaling can be performed in a single composition or multiple compositions, separately or at the same time. Several unit dosage forms may be administered at about the same time. A dose employed may be determined by a physician or qualified medical professional, and depends upon the desired therapeutic effect, the route of administration and the duration of the treatment, and the condition of the patient.

The term “dose” includes, but is not limited to an effective dose, such as, for example, an acute dose, a sub-acute dose, and a chronic or continuous dose.

The terms “acute dose” or “acute administration” of one or more active agents mean the scheduled administration of the active agent(s) to a patient on an as-needed basis at a dosage level determined by the attending physician to elicit a relatively immediate desired reaction in the patient, given the patient's age and general state of health.

A “sub-acute dose” is a dose of the active agent(s) at a lower level than that determined by the attending physician to be required for an acute dose, as described above. Sub-acute doses may be administered to the patient on an as-needed basis, or in a chronic, or on-going dosing regimen.

The terms “chronic dose” or “continuous administration” of the active agent(s) mean the scheduled administration of the active agent(s) to the patient on an on-going day-to-day basis.

An effective dose will generally depend upon the factors described above. Generally, oral, nasal, intravenous, intracerebroventricular, subcutaneous, and inhalation doses of the small molecule inhibitors of Wnt- and TGF-β-mediated β-catenin signaling for a subject, will range from about 0.000001 to about 1000 mg per kilogram, about 0.000005 to about 950 mg per kilogram, about 0.00001 to about 850 mg per kilogram, about 0.00005 to about 750 mg per kilogram, about 0.0001 to about 500 mg per kilogram, about 0.0005 to about 250 mg per kilogram, about 0.001 to about 100 mg per kilogram, about 0.001 to about 50 mg per kilogram, about 0.001 to about 25 mg per kilogram, about 0.001 to about 10 mg per kilogram, about 0.001 to about 1 mg per kilogram, about 0.005 to about 100 mg per kilogram, about 0.005 to about 50 mg per kilogram, about 0.005 to about 25 mg per kilogram, about 0.005 to about 10 mg per kilogram, about 0.005 to about 1 mg per kilogram, about 0.01 to about 100 mg per kilogram, about 0.01 to about 50 mg per kilogram, about 0.01 to about 25 mg per kilogram, about 0.01 to about 10 mg per kilogram, about 0.01 to about 1 mg per kilogram, about 0.05 to about 50 mg per kilogram, about 0.05 to about 25 mg per kilogram, about 0.05 to about 10 mg per kilogram, about 0.05 to about 1 mg per kilogram, about 0.1 to about 25 mg per kilogram, about 0.1 to about 10 mg per kilogram, about 0.1 to about 1 mg per kilogram, about 0.1 to about 0.5 mg per kilogram of body weight per day.

In another embodiment, one or more small molecule inhibitors of Wnt- and TGF-β-mediated β-catenin signaling is administered orally, by inhalation, nasally, or parenterally to a subject at a dose of about 0.25 to 3 g per kg, about 0.5 to 2.5 g per kg, about 1 to 2 g per kg, about 1.25 to 1.75 g per kg or about 1.5 g per kg of bodyweight per day.

In particular embodiments, one or more small molecule inhibitors of Wnt- and TGF-β-mediated β-catenin signaling is administered orally, by inhalation, nasally, or parenterally to a subject at a dose of about 10 g per kg, about 0.25 g per kg, about 0.50 g per kg, about 0.75 g per kg, about 1.0 g per kg, about 1.25 g per kg, about 1.50 g per kg, about 1.75 g per kg, or about 2.00 g per kg of bodyweight per day.

In other related embodiments, one or more small molecule inhibitors of Wnt- and TGF-β-mediated β-catenin signaling is administered orally, by inhalation, nasally, or parenterally to a subject at a dose of about 0.01 μg to 1 mg per kg, about 0.1 to 100 μg per kg, or about 1 to 10 μg per kg or any increment of concentration in between. For example, in particular embodiments, one or more small molecule inhibitors of Wnt- and TGF-β-mediated β-catenin signaling is administered orally nasally, or parenterally to a subject at a dose of about 1 μg per kg, about 2 μg per kg, about 3 μg per kg, about 4 μg per kg, about 5 μg per kg, about 6 μg per kg, about 7 μg per kg, about 8 μg per kg, about 9 μg per kg, or about 10 μg per kg.

In particular embodiments, one or more small molecule inhibitors of Wnt- and TGF-β-mediated β-catenin signaling is administered orally, by inhalation, nasally, or parenterally to a subject at a dose of about 0.005 μg per kg, about 0.01 μg per kg, about 1.0 μg per kg, about 10 μg per kg, about 50 μg per kg, about 100 μg per kg, about 250 μg per kg, about 500 μg per kg, or about 1000 μg per kg

A composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Moreover, multiple administrations of the same or different compositions of the present invention may be administered, multiples times, for extended periods of time, as noted above.

In particular embodiments, the frequency of delivery of a composition is once a day, twice a day, three times day, four times a day, once every two days, or once a week or any intervening frequency.

In particular embodiments, the duration of continuous delivery of a composition is between 30 seconds and 24 hours, between 30 seconds and 12 hours, between 30 seconds and 8 hours, between 30 seconds and 6 hours, between 30 seconds and 4 hours, between 30 seconds and 2 hours, between 30 seconds and 1 hour, between 30 seconds and 30 minutes, between 30 seconds and 15 minutes, between 30 seconds and 10 minutes, between 30 seconds and 5 minutes, between 30 seconds and 2 minutes, between 30 seconds and 1 minute or any intervening period of time.

Additional methods of formulating compositions known to the skilled artisan, for example, as described in the Physicians Desk Reference, 62nd edition. Oradell, N.J.: Medical Economics Co., 2008; Goodman & Gilman's The Pharmacological Basis of Therapeutics, Eleventh Edition. McGraw-Hill, 2005; Remington: The Science and Practice of Pharmacy, 20th Edition. Baltimore, Md.: Lippincott Williams & Wilkins, 2000; and The Merck Index, Fourteenth Edition. Whitehouse Station, N.J.: Merck Research Laboratories, 2006; are hereby incorporated by reference in relevant parts

### G. Kits

In various illustrative embodiments, the present invention contemplates, in part, to provide a kit comprising one or more small molecule inhibitors of Wnt- and TGF-β-mediated β-catenin signaling in a pharmaceutical composition suitable for treating fibrosis or fibroproliferative disease. In a particular embodiment, a pharmaceutical composition comprising one or more small molecule inhibitors of Wnt- and TGF-β-mediated β-catenin signaling can be provided in a kit. In one embodiment, the kit includes (a) a container that contains a composition that includes one or more small molecule inhibitors of Wnt- and TGF-β-mediated β-catenin signaling and, optionally (b) informational material. The informational material can be descriptive, instructional, marketing or other material that relates to the methods described herein and/or the use of the small molecules for therapeutic benefit.

The informational material of the kits is not limited in its form. In a particular embodiment, the informational material can include information about production of the compound, molecular weight of the compound, concentration, date of expiration, batch or production site information, and so forth. In another particular embodiment, the informational material relates to methods of administering the pharmaceutical composition comprising one or more small molecule inhibitors of Wnt- and TGF-β-mediated β-catenin signaling, e.g., in a suitable amount, manner, or mode of administration (e.g., a dose, dosage form, or mode of administration described herein). The method can be a method of treating fibrosis or a fibroproliferative disease, as described herein.

The informational material can provide instructions provided in printed matter, e.g., a printed text, drawing, and/or photograph, e.g., a label or printed sheet. However, the informational material can also be provided in other formats, such as Braille, computer readable material, video recording, or audio recording. In another particular embodiment, the informational material of the kit is contact information, e.g., a physical address, email address, website, or telephone number, where a user of the kit can obtain substantive information about small molecules therein and/or its use in the methods described herein. Of course, the informational material can also be provided in any combination of formats.

In addition to a small molecule inhibitor of Wnt- and TGF-β-mediated β-catenin signaling, the composition of the kit can include other ingredients, such as a solvent or buffer, a stabilizer, or a preservative. The kit can also include other (e.g., 1, 2, 3, 4, or 5) therapeutic agents.

The small molecule inhibitors can be provided in any form, e.g., liquid, dried or lyophilized form. It is preferred that the agents are substantially pure (although they can be combined together or delivered separate from one another) and/or sterile. When the small molecule inhibitors are provided in a liquid solution, the liquid solution preferably is an aqueous solution, with a sterile aqueous solution being preferred. When the small molecule inhibitors are provided as a dried form, reconstitution generally is by the addition of a suitable solvent. The solvent, e.g., sterile water or buffer, can also be provided in the kit.

Kits contemplated in particular embodiments of the present invention can comprise one or more containers for the composition or compositions containing the small molecule inhibitors of Wnt- and TGF-β-mediated β-catenin signaling. In certain embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. The containers can include a unit dosage, e.g., a unit that includes the small molecule inhibitor of Wnt- and TGF-β-mediated β-catenin signaling. For example, the kit includes a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit optionally includes a device suitable for administration of the composition, e.g., a syringe, inhaler, nebulizer, or other suitable delivery device. The device can be provided pre-loaded with one or more small molecule inhibitors of Wnt- and TGF-β-mediated β-catenin signaling, e.g., in a unit dose, or can be empty, but suitable for loading.

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

The present inventors sought to indentify small molecule inhibitors of Wnt- and TGF-β-mediated β-catenin signaling that could be used for the prevention, amelioration, and/or treatment of fibrosis and fibroproliferative disease. Accordingly, a small molecule library was screened for compounds that would inhibit both Wnt- and TGF-β-mediated β-catenin signaling in an in vitro cell culture assay using a human lung epithelial cell line and primary mouse alveolar cells.

### Example 1

### Identification of Wnt- and TGF-b-Mediated b-Catenin Signaling Antagonists in a Human Lung Epithelial Cell Line

A human lung epithelial cell line (A549; obtained from ATCC) was stably infected with a lentiviral β-catenin reporter construct (pBARL), to allow β-catenin signaling to be measured. β-catenin is known to regulate transcription in combination with TCF and LEF transcription factors. Accordingly, the 6-catenin report construct included a multimerized motif of 12 TCF/LEF DNA binding sites as a transcriptional control element to drive firefly luciferase expression. A549 cells were stably transduced with the lentiviral β-catenin reporter and with a Renilla luciferase construct comprising a constitutive promoter. The constitutive expression of Renilla luciferase served as a normalization tool for the firefly luciferase activity measurements. The resultant cell line was referred to as A549/pBARL.

A library of small molecule compounds was screened to identify those compounds that inhibited both Wnt- and TGF-β-mediated β-catenin in A549/pBARL cells. A549/pBARL cells were seeded at 3000 cells/well in 30 microliters of F12 medium containing 10% heat inactivated fetal bovine serum, 3.0 ug per milliliter puromycin, and 400 ug per milliliter hygromycin in 384 well plates. One day after plating, the cells were treated with 40 nanoliters of test compound in a six-point dose response ranging from a concentration of 10 micromolar to 30 nanomolar, with each dose being a half-log dilution of the previous concentration.

The ability of the compounds to inhibit Wnt-mediated β-catenin signaling was tested by adding 10 microliters of Wnt3a conditioned media (final dilution 1:8) immediately after the last test compound was added to a 384 well plate. The ability of the compounds to inhibit TGF-β-mediated β-catenin signaling was tested in a corresponding plate; the plating medium was replaced with SABM media (Lonza) containing 1 milligram per milliliter human serum albumin and TGF-β1 was added to final concentration of 3 nanograms per milliliter immediately after the last test compound was added to a duplicate 384 well plate.

Cells were incubated with test compound and either Wnt3a conditioned media or TGF-β1 for 20 hours before being assayed for firefly luciferase and Renilla luciferase levels with Promega's Dual Glo® reagent and a Perkin Elmer Envision® multilabel microplate reader. IC50 values were calculated from dose-response curve data generated by measuring the ratio of firefly luciferase to Renilla luciferase at different concentrations of small molecule test compound.

FIGS. 1-114 show the structure of the compounds determined to be small molecule inhibitors of Wnt- and TGF-β-mediated β-catenin signaling, the compound name, dose-response curve data, and IC₅₀s that were measured using the foregoing assay. In addition, Table 1 shows the name of the small molecule inhibitors of Wnt- and TGF-β-mediated β-catenin signaling and IC₅₀s of the compounds shown in FIGS. 1-114.

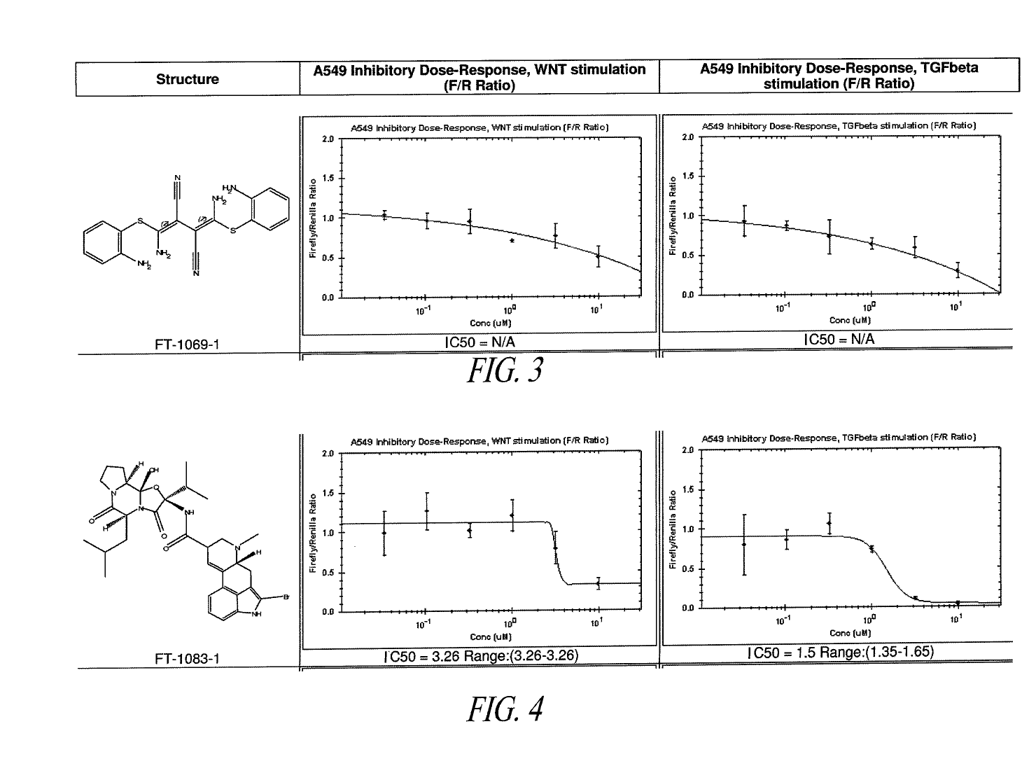

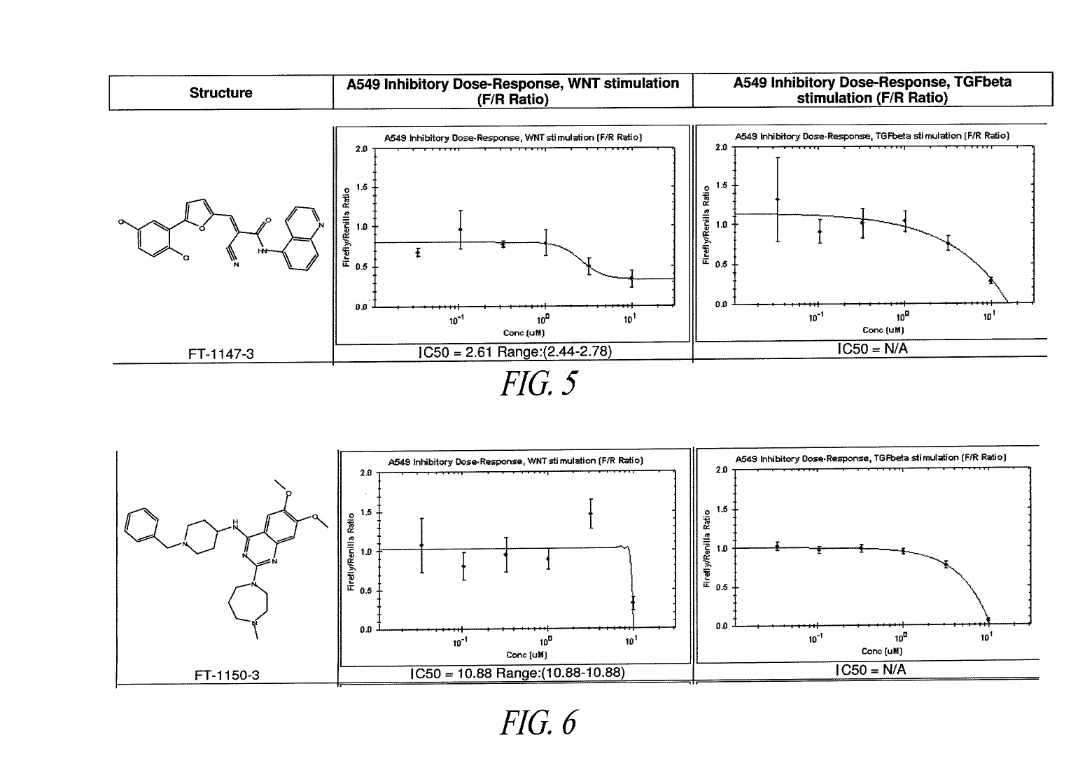

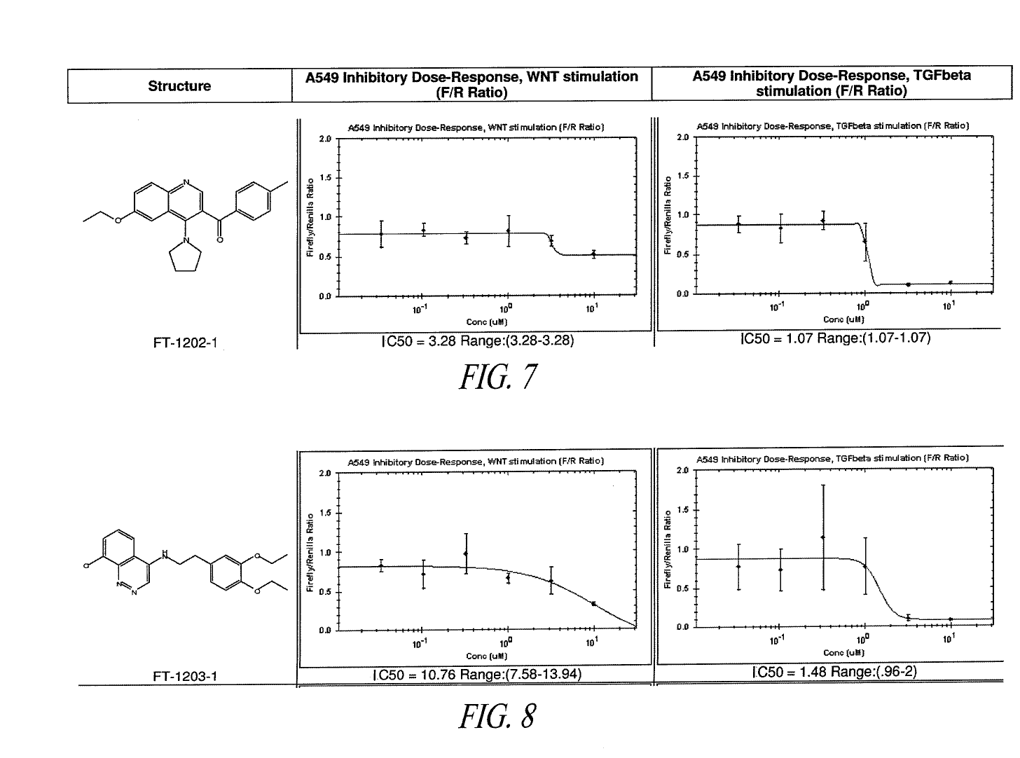

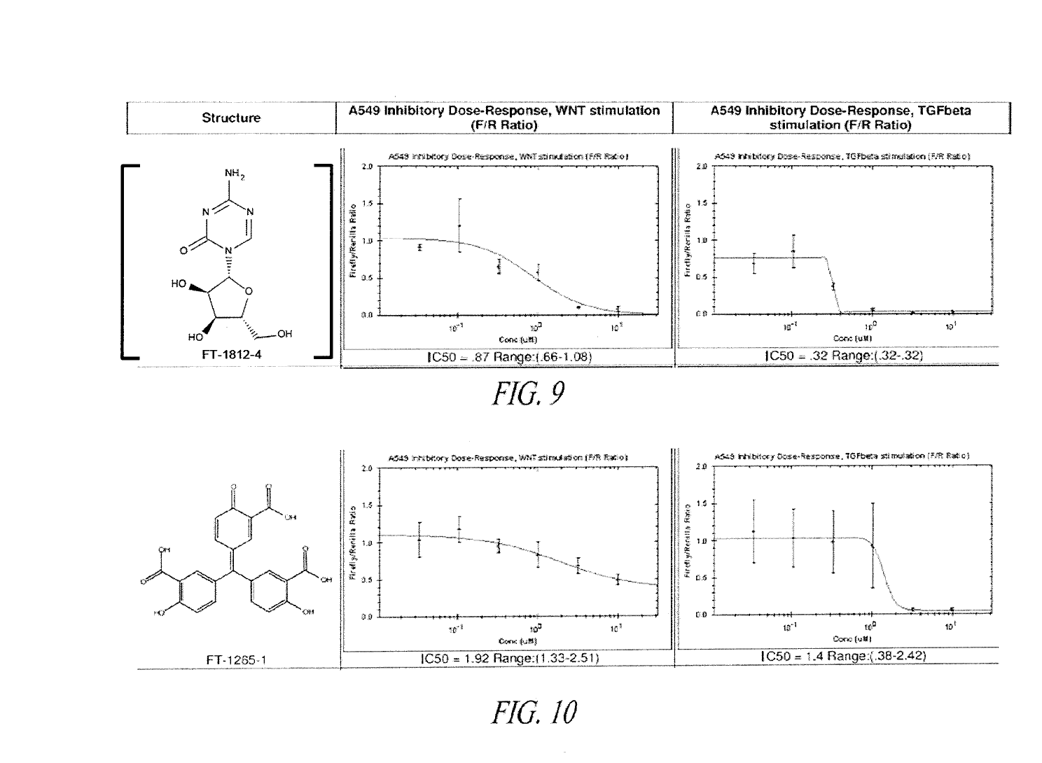

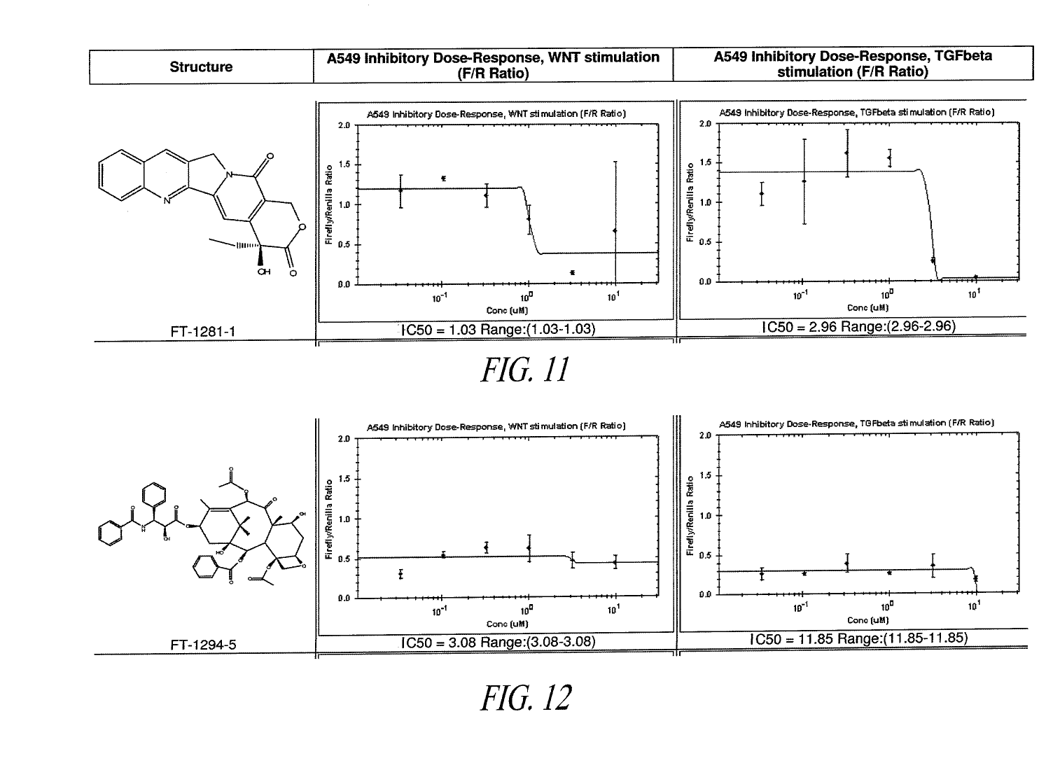

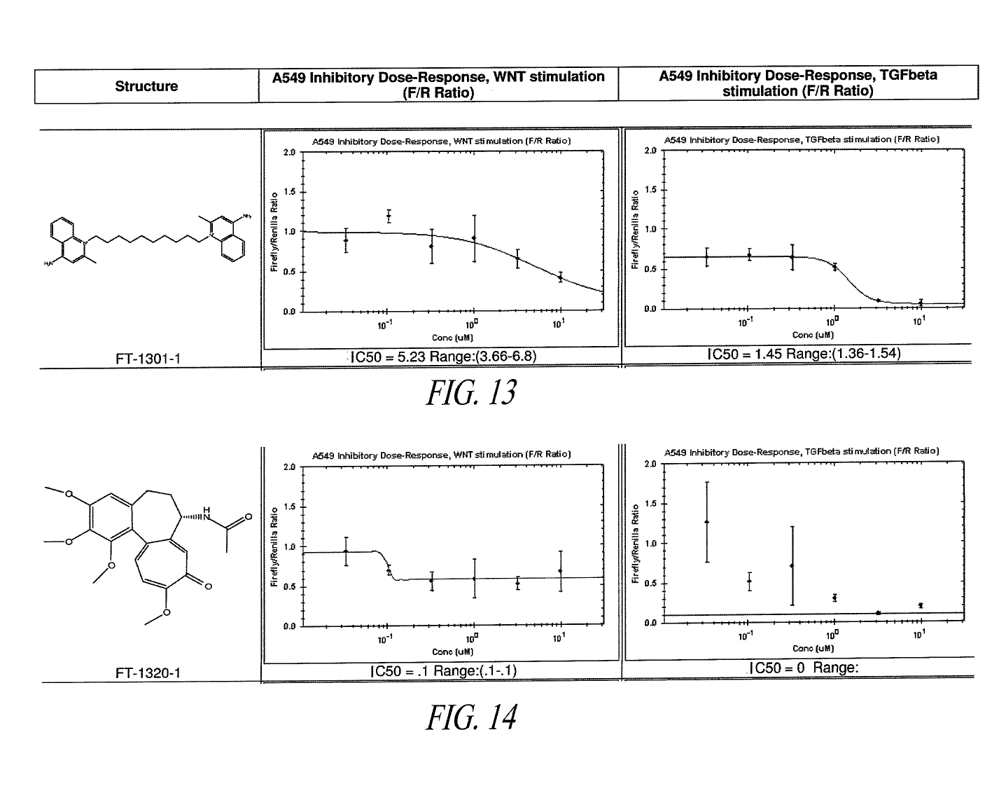

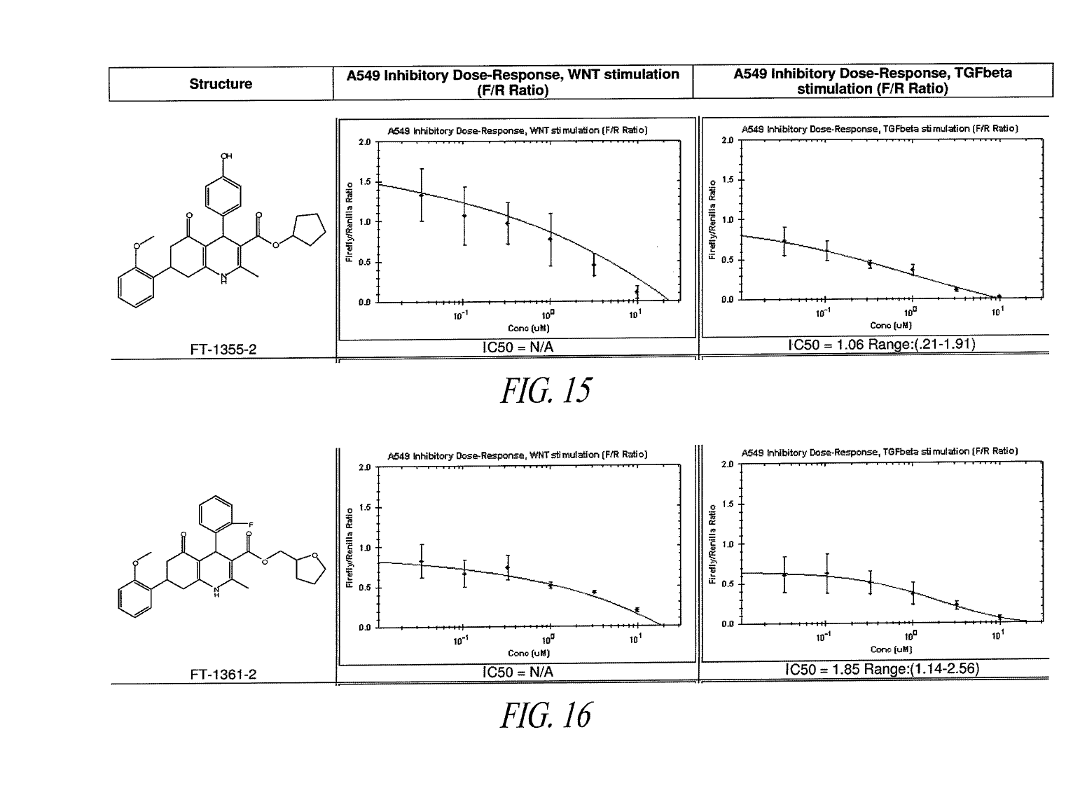

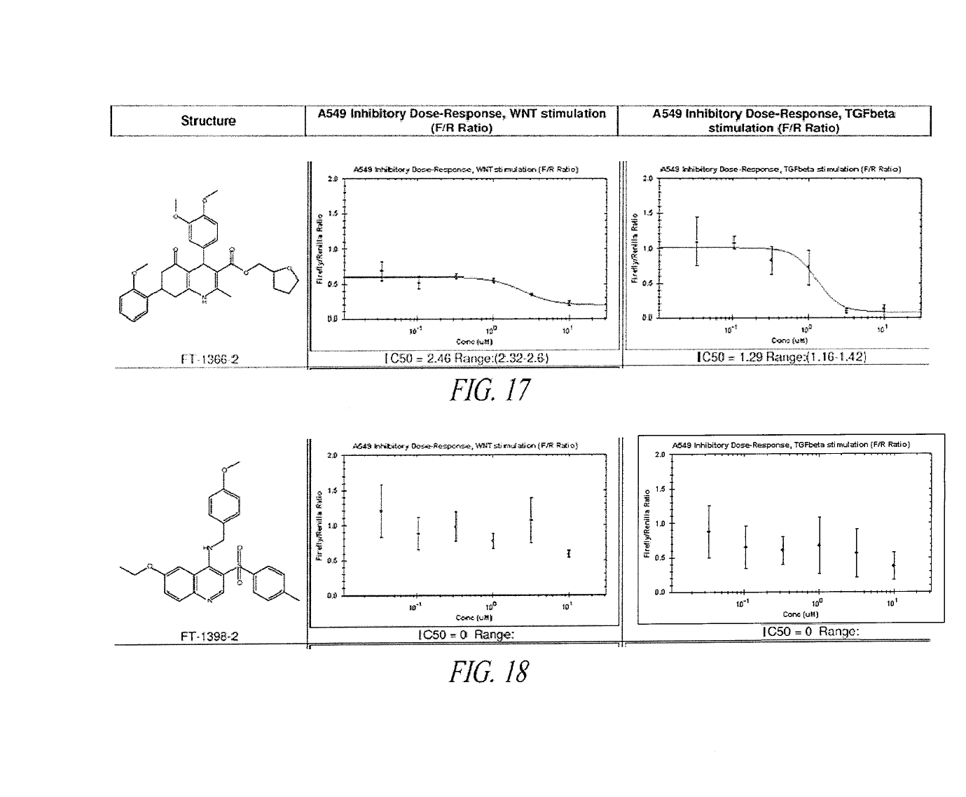

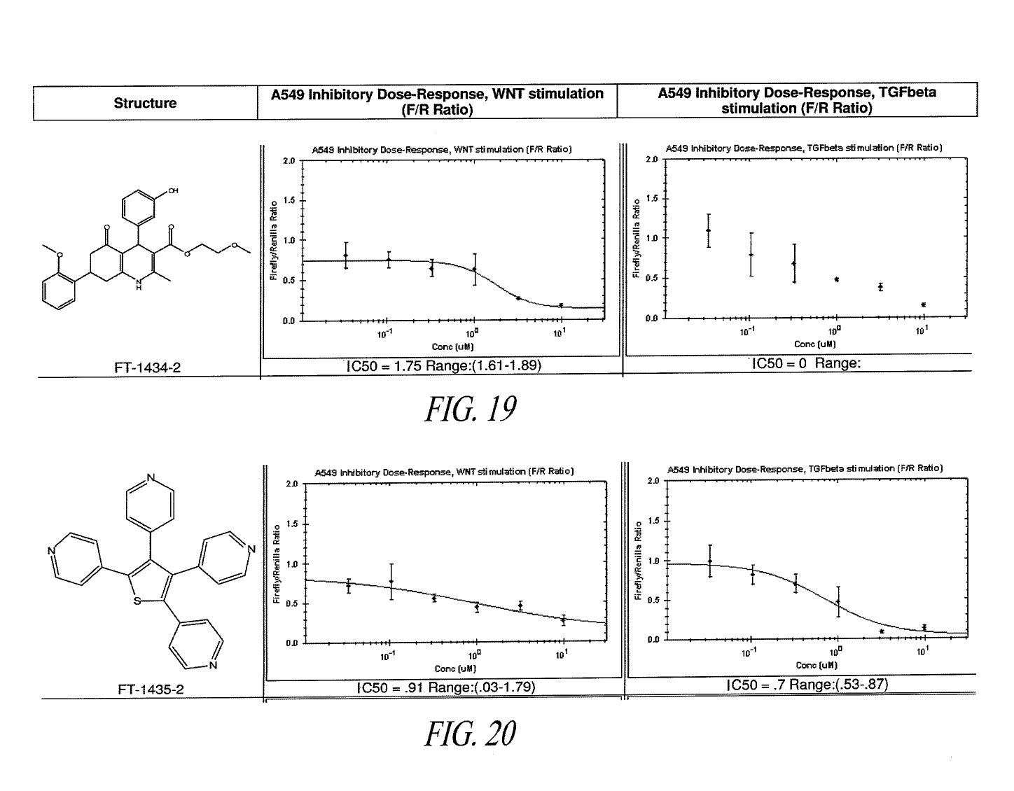

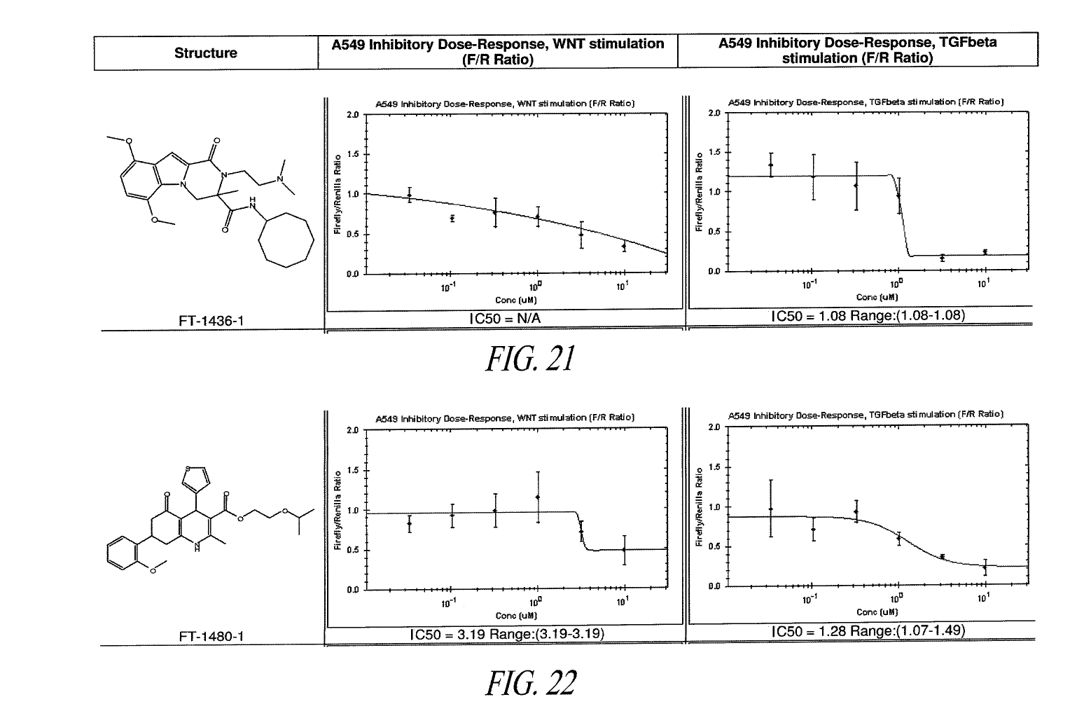

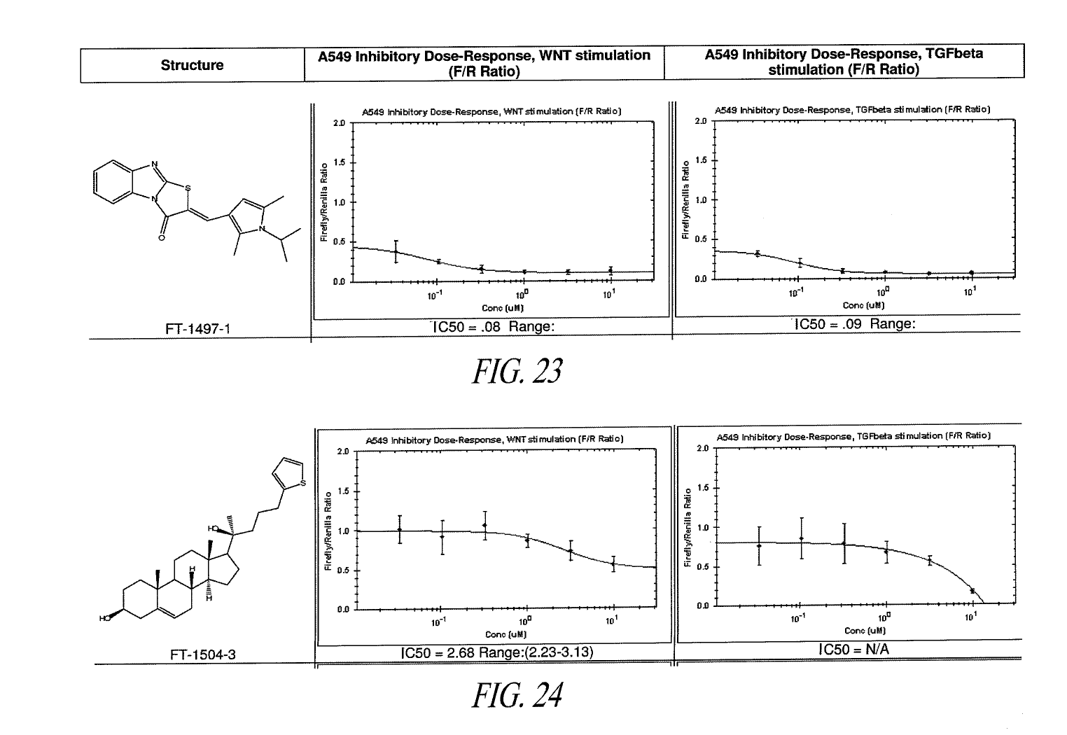

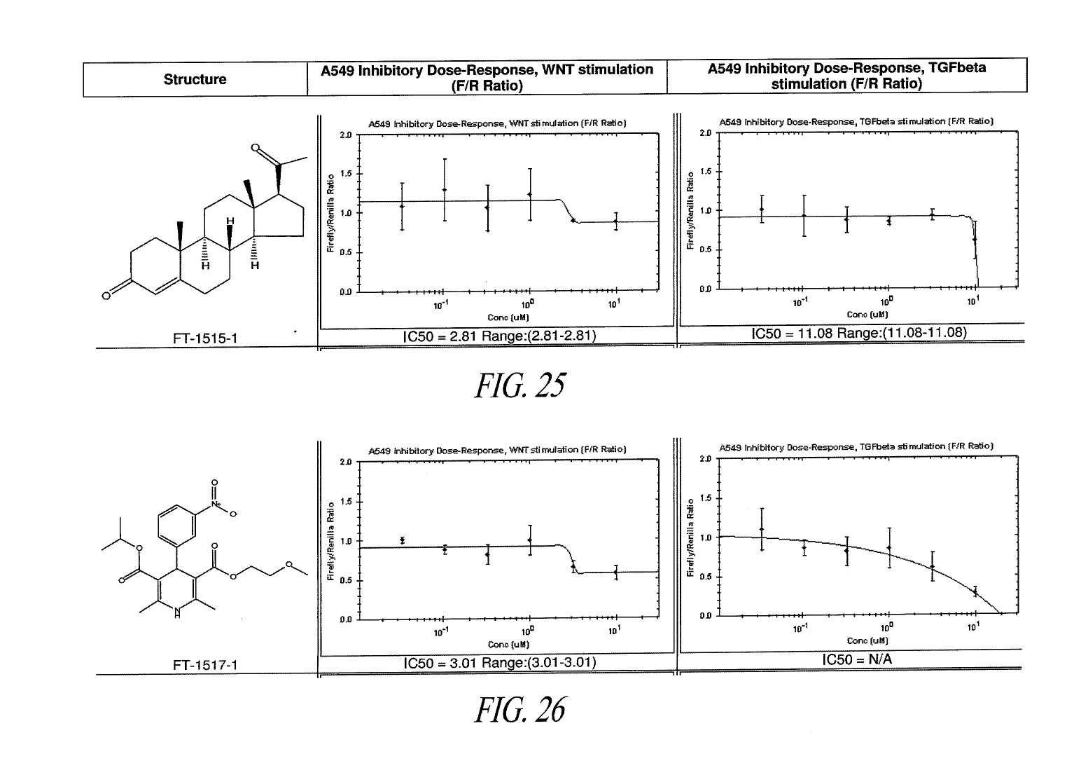

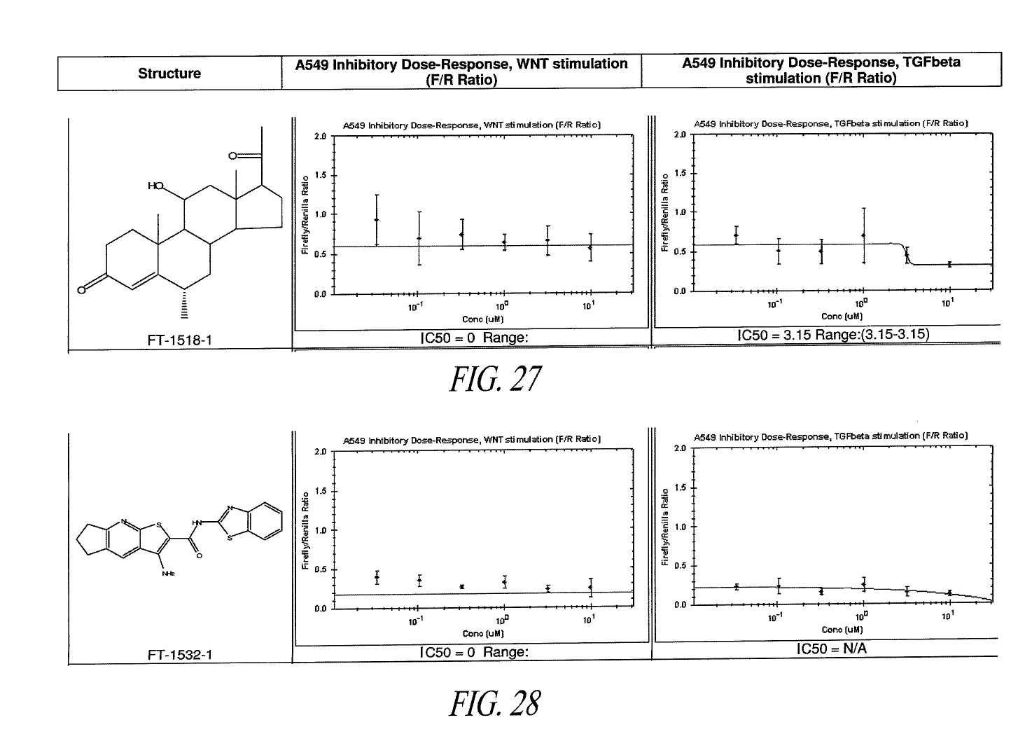

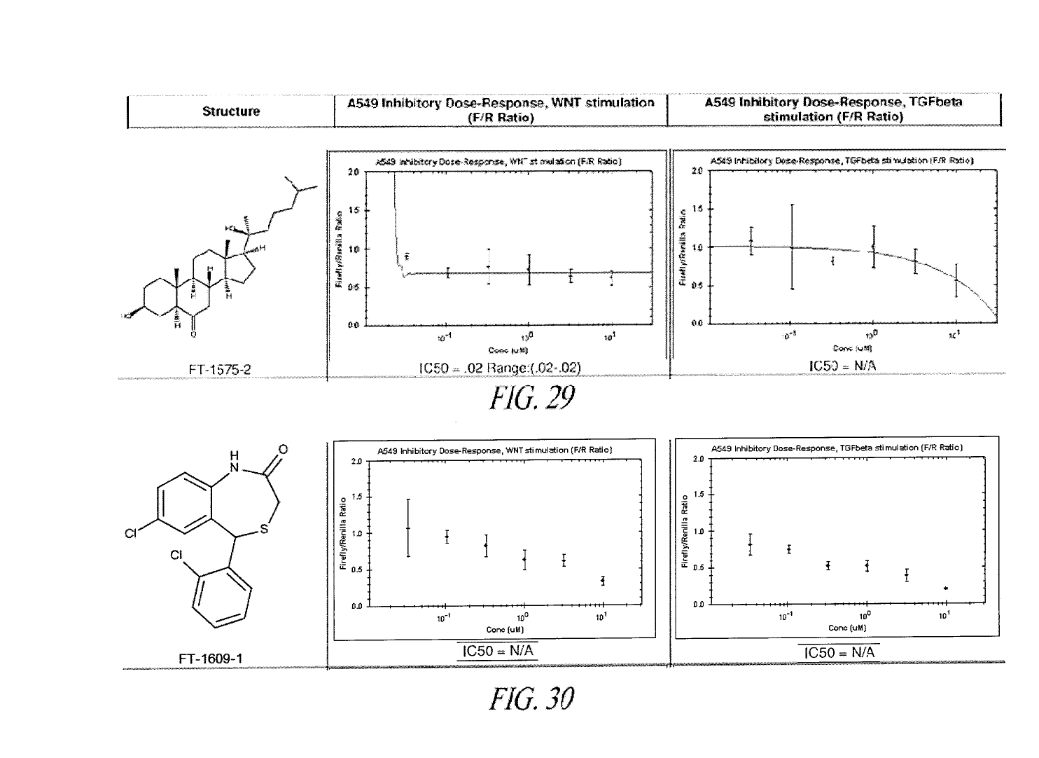

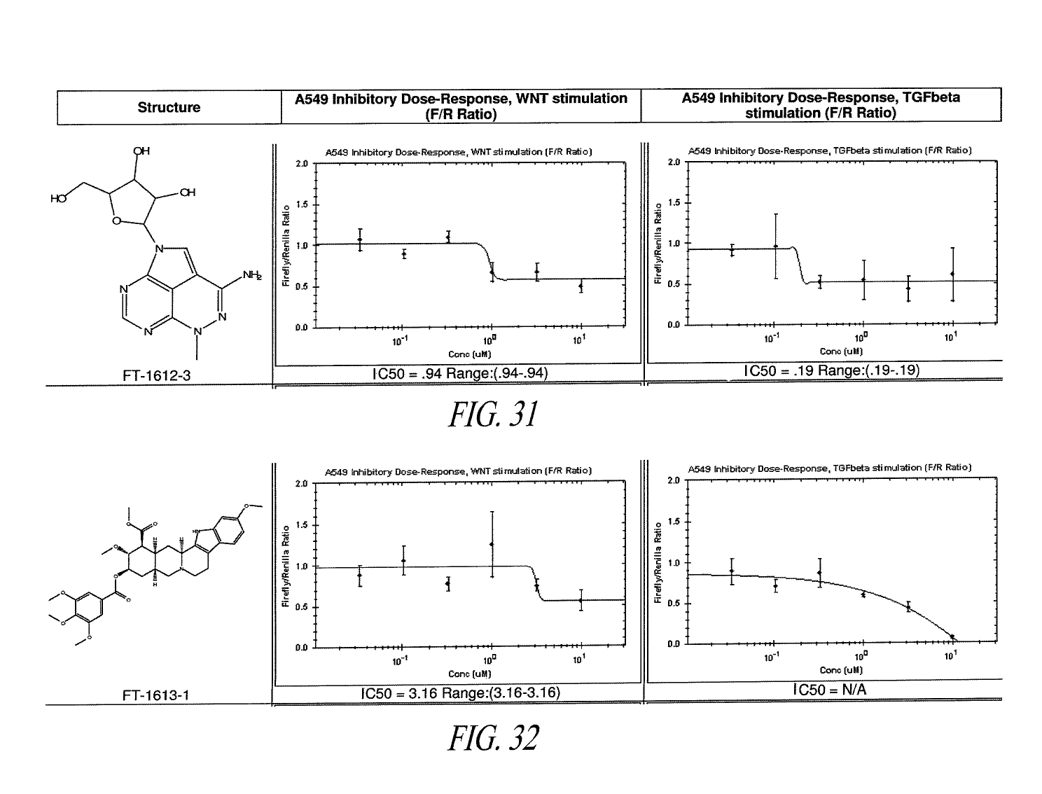

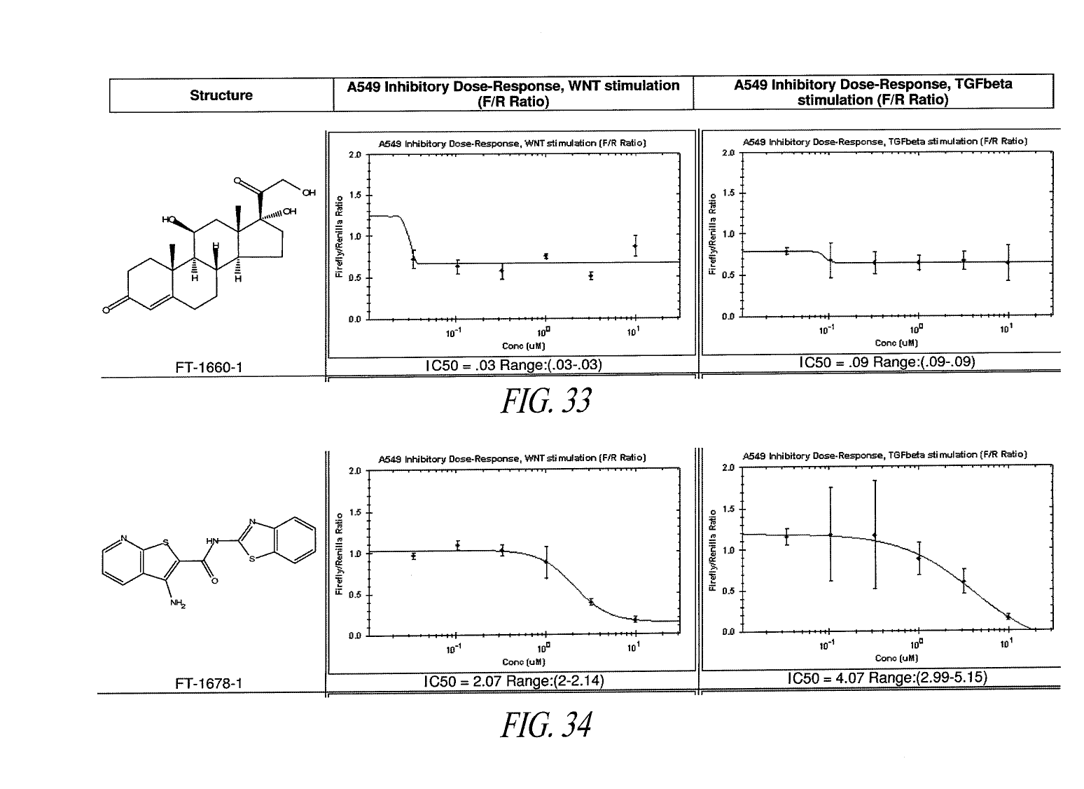

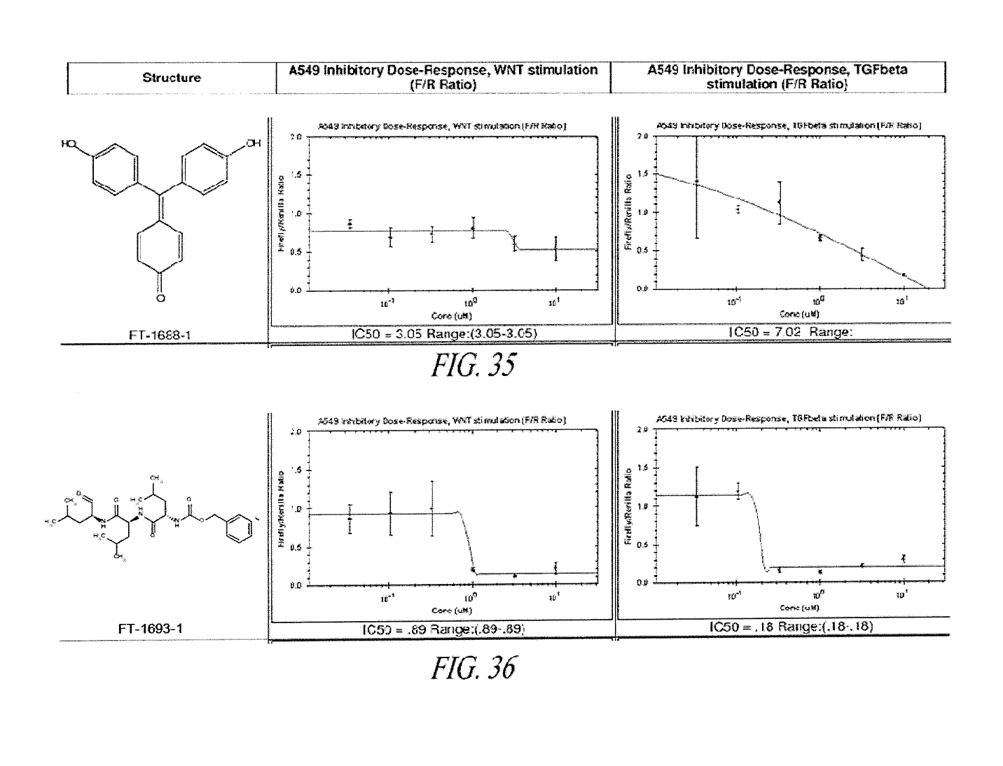

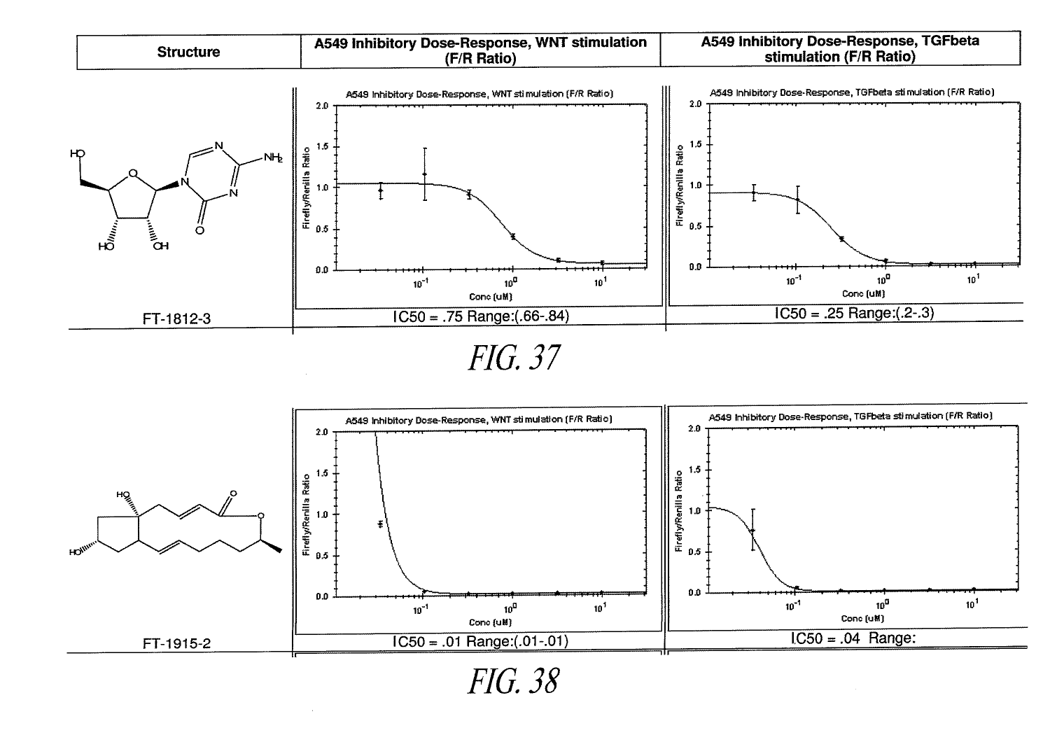

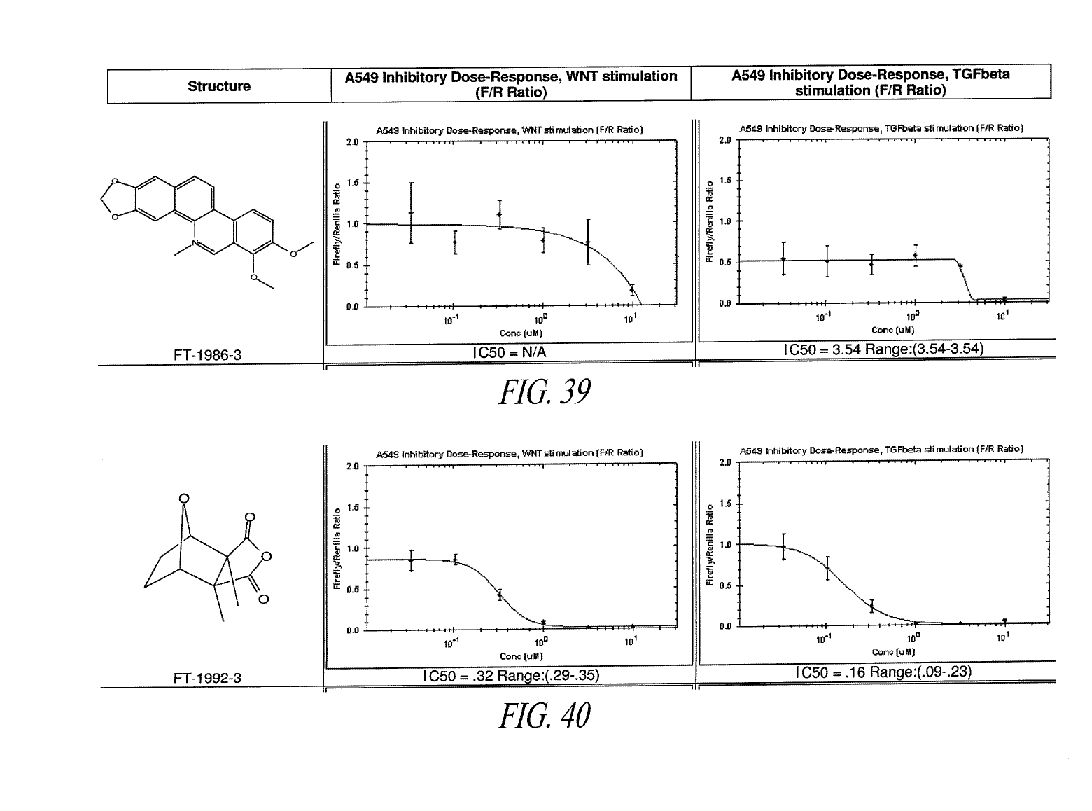

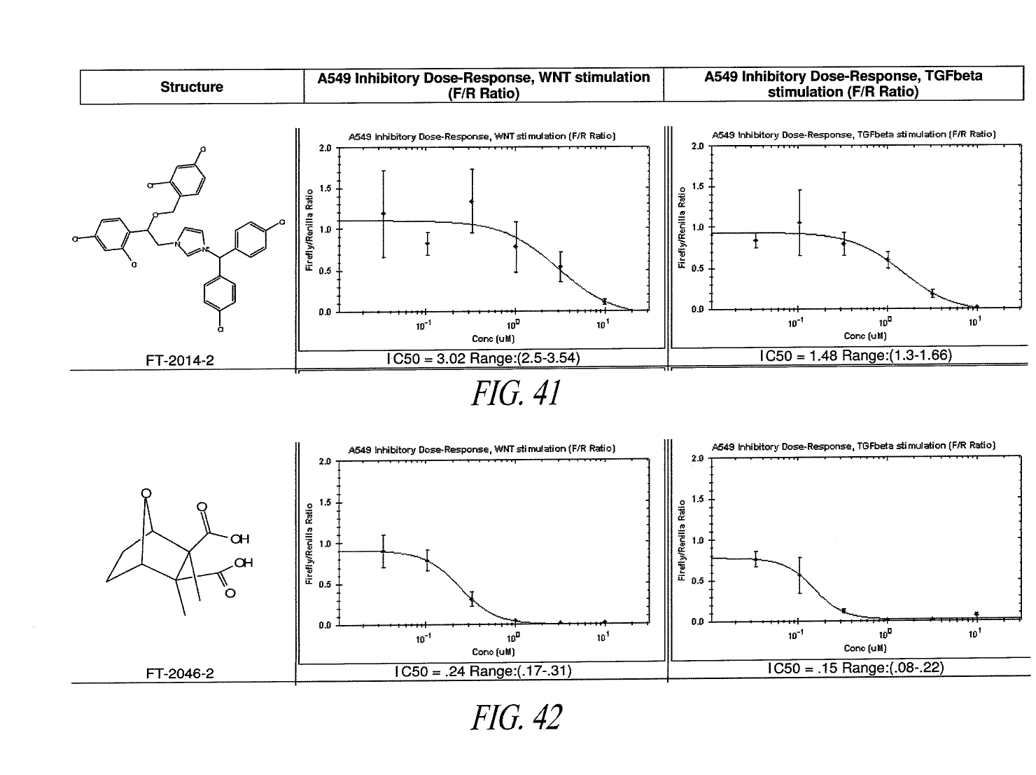

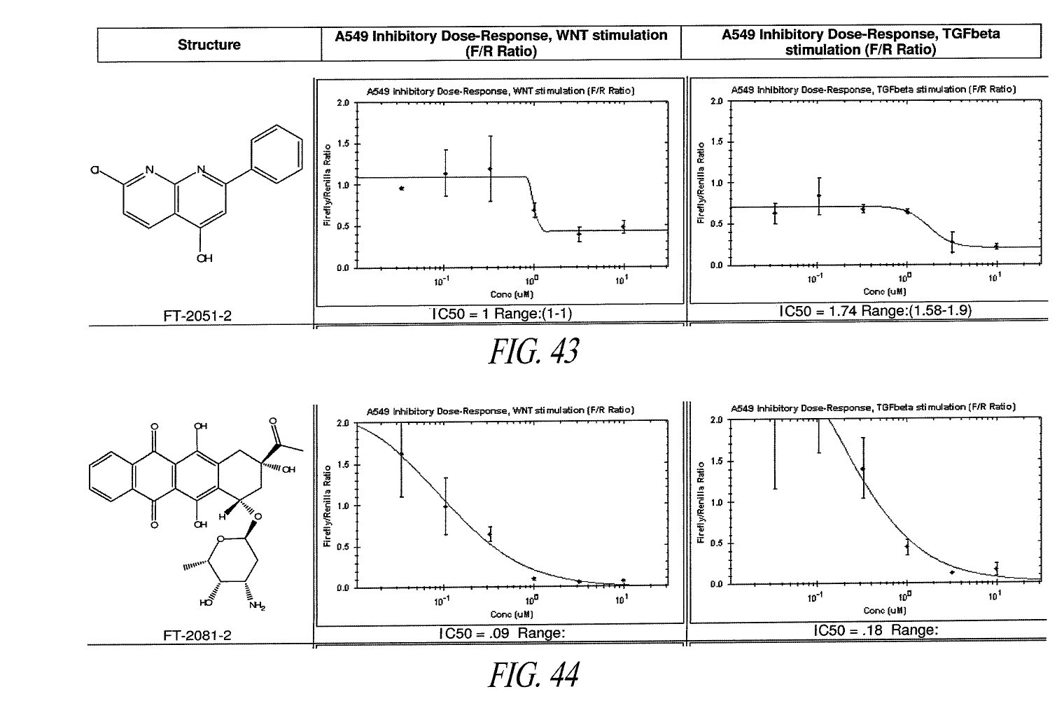

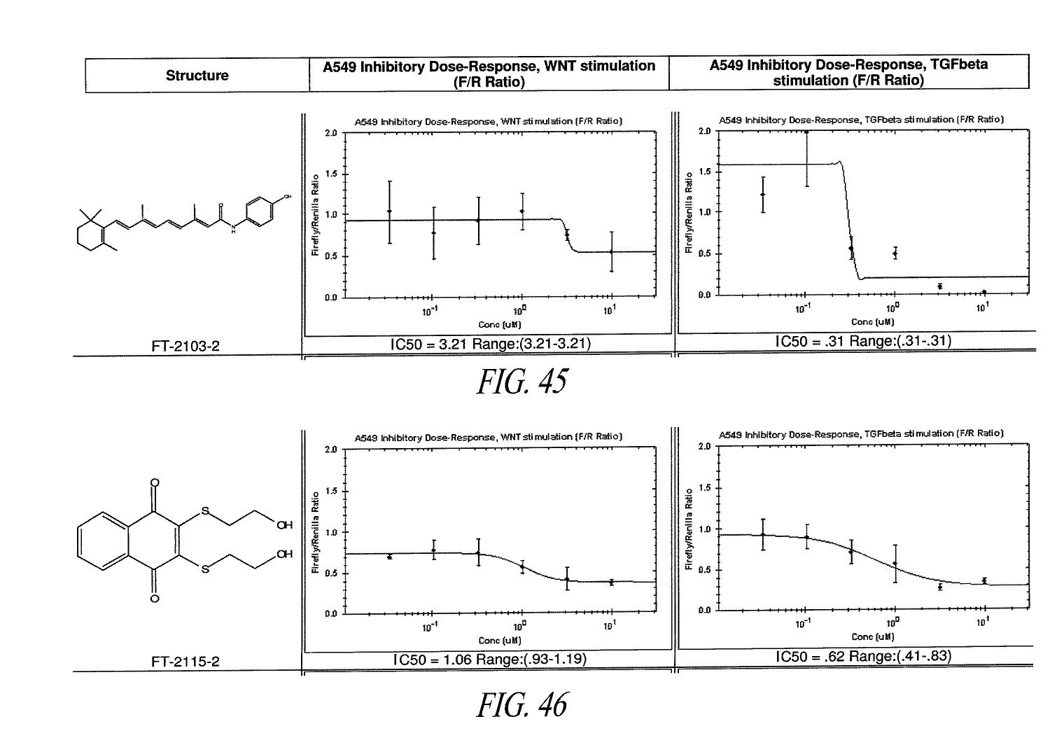

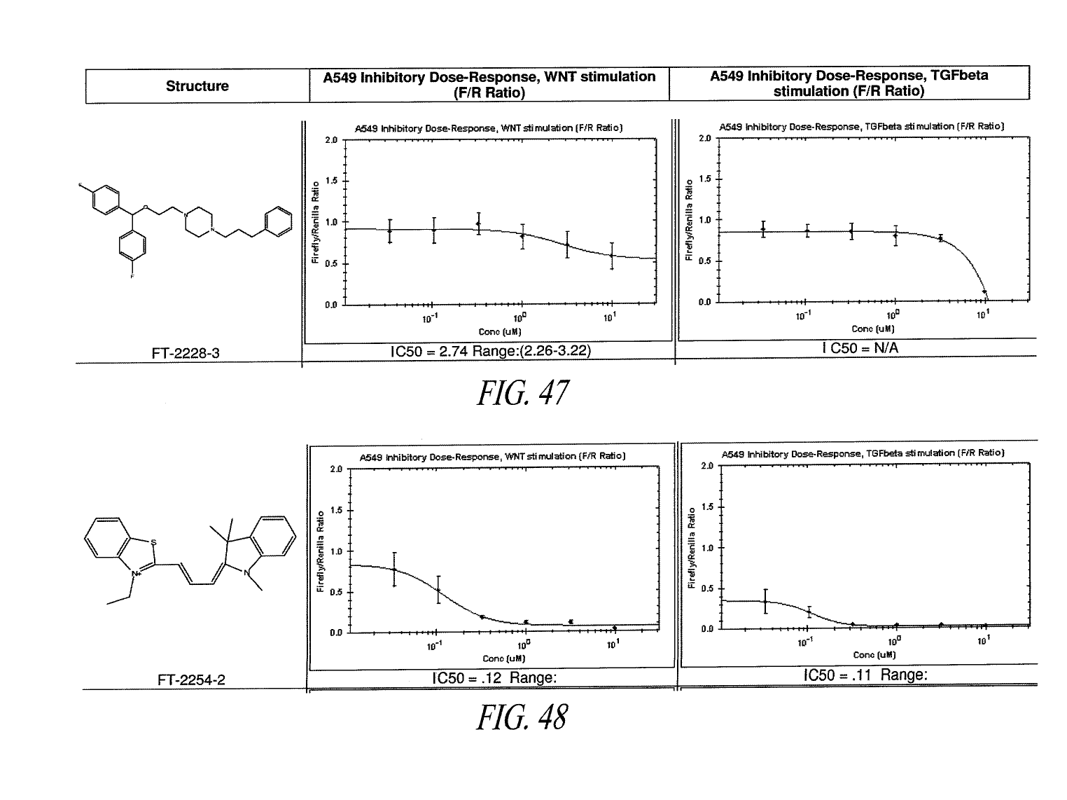

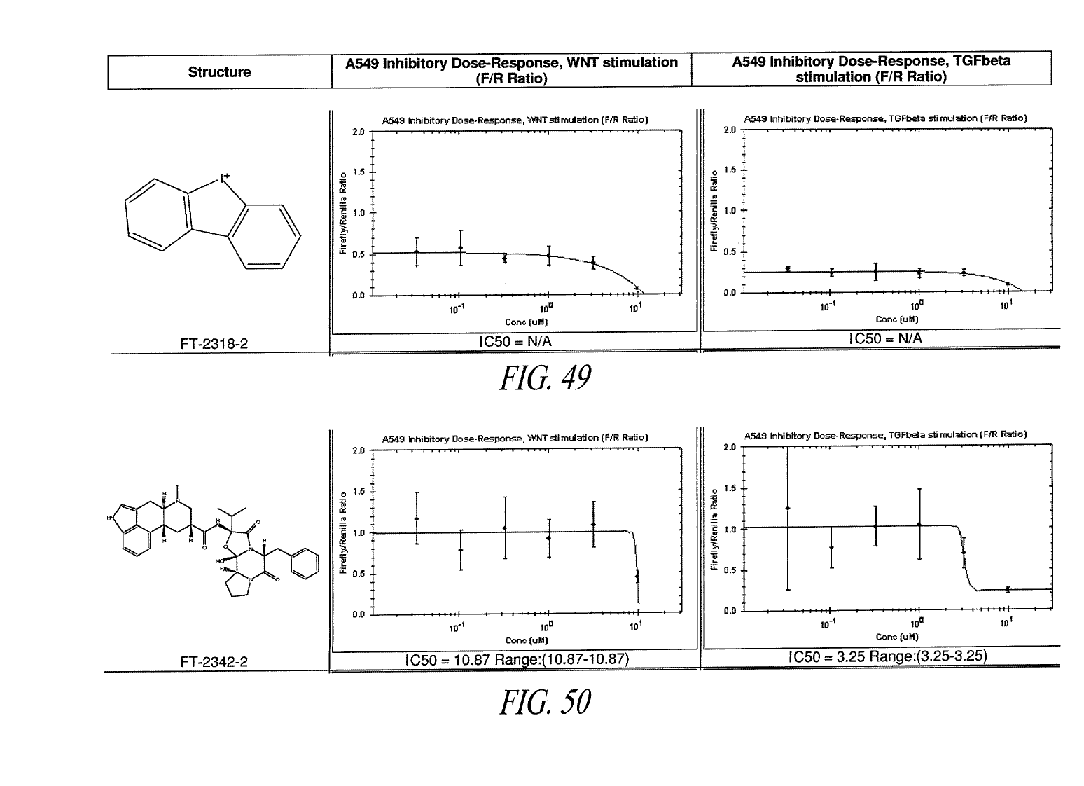

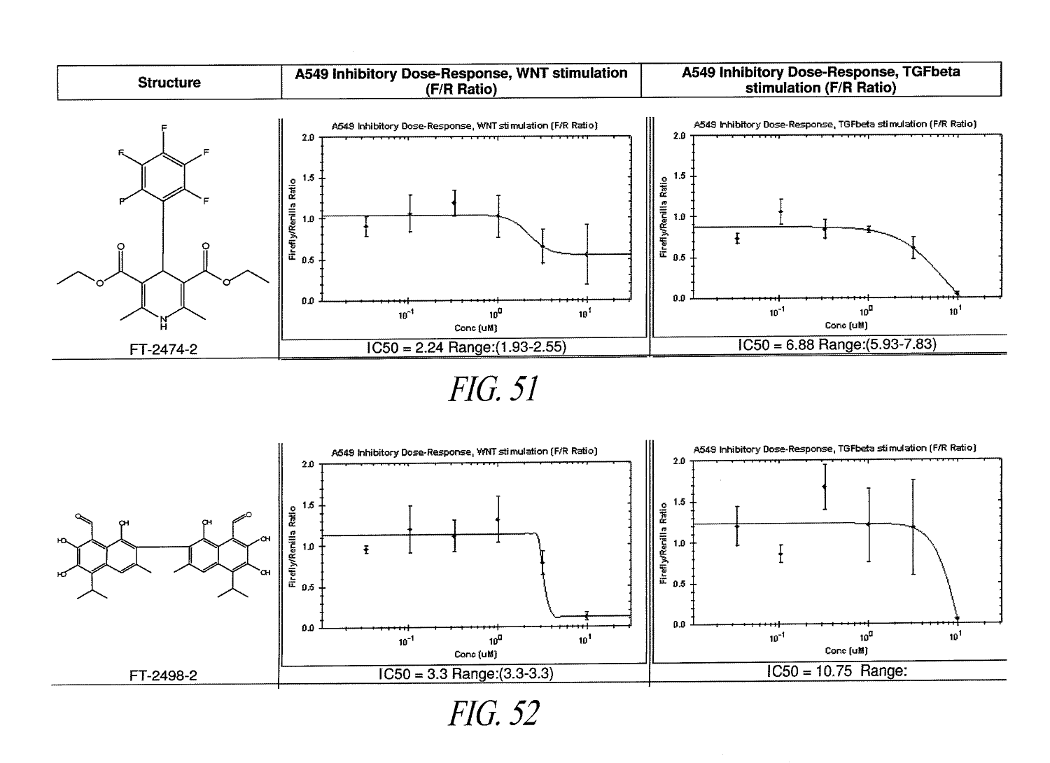

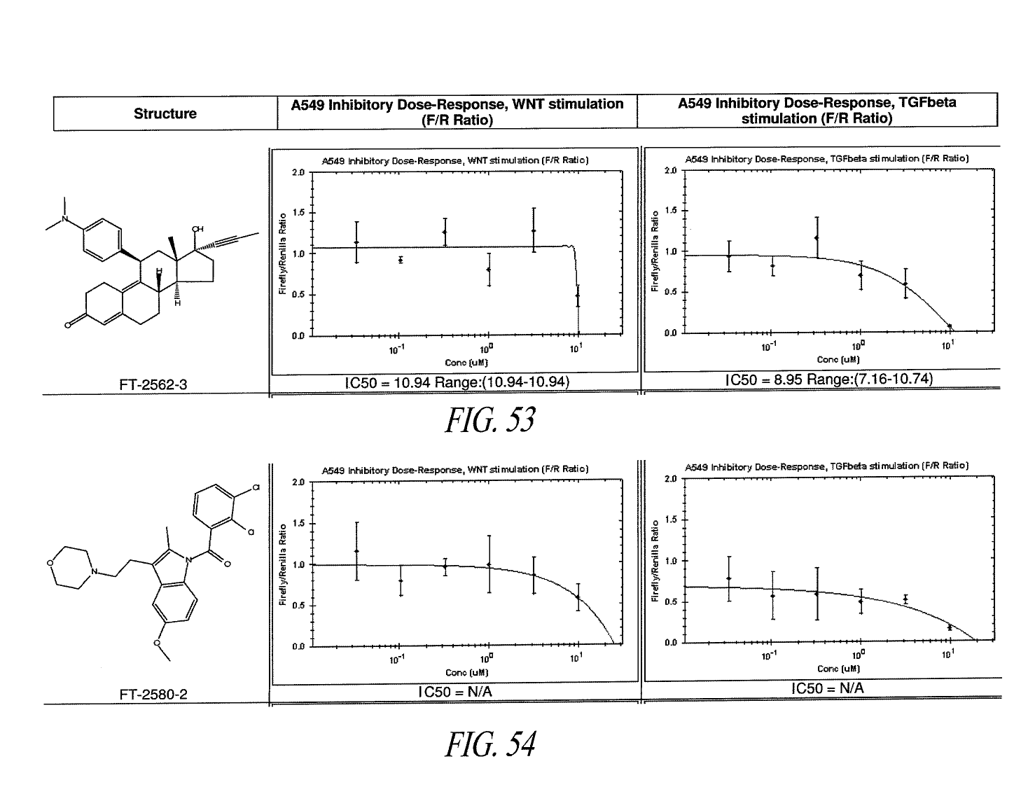

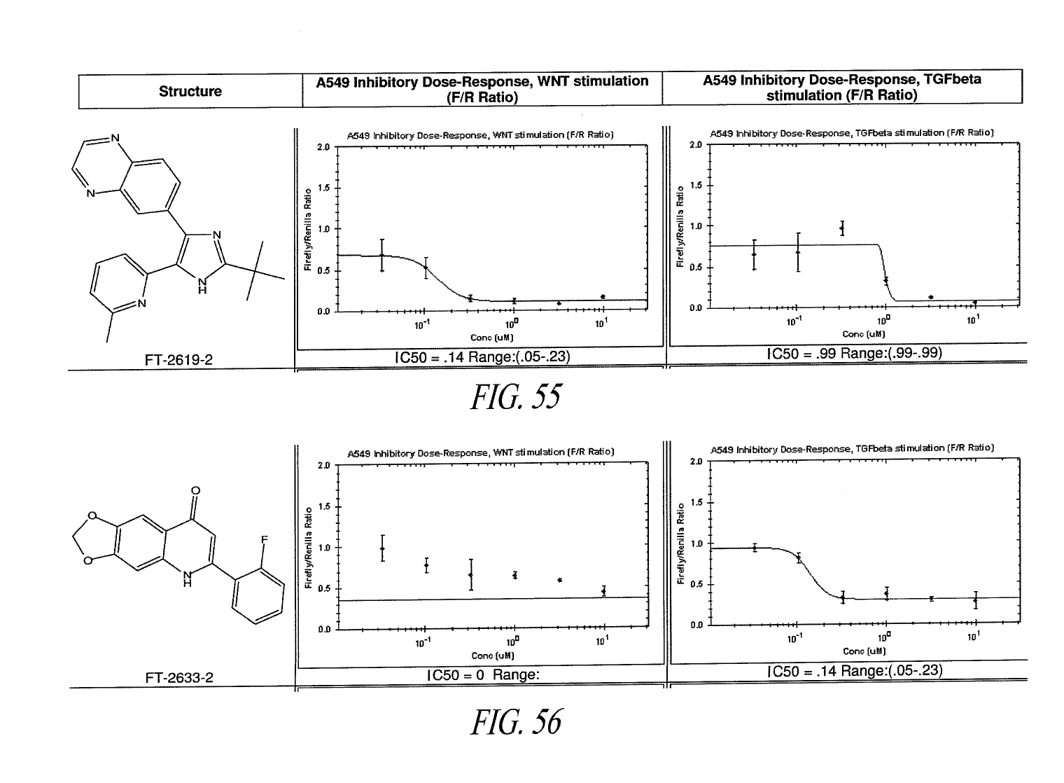

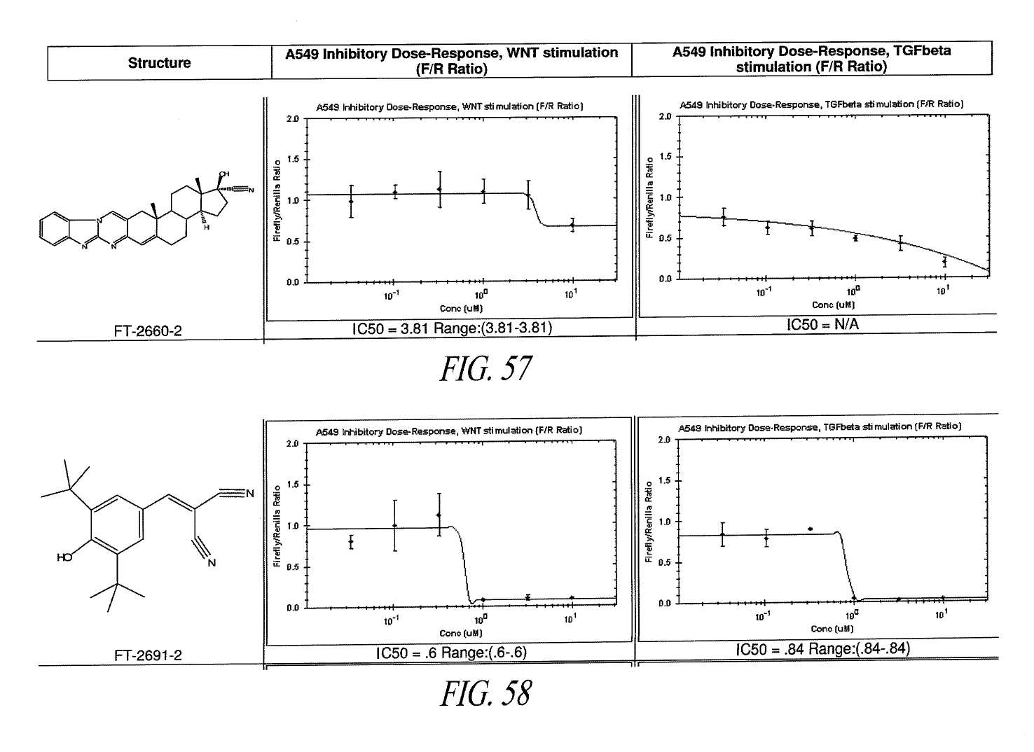

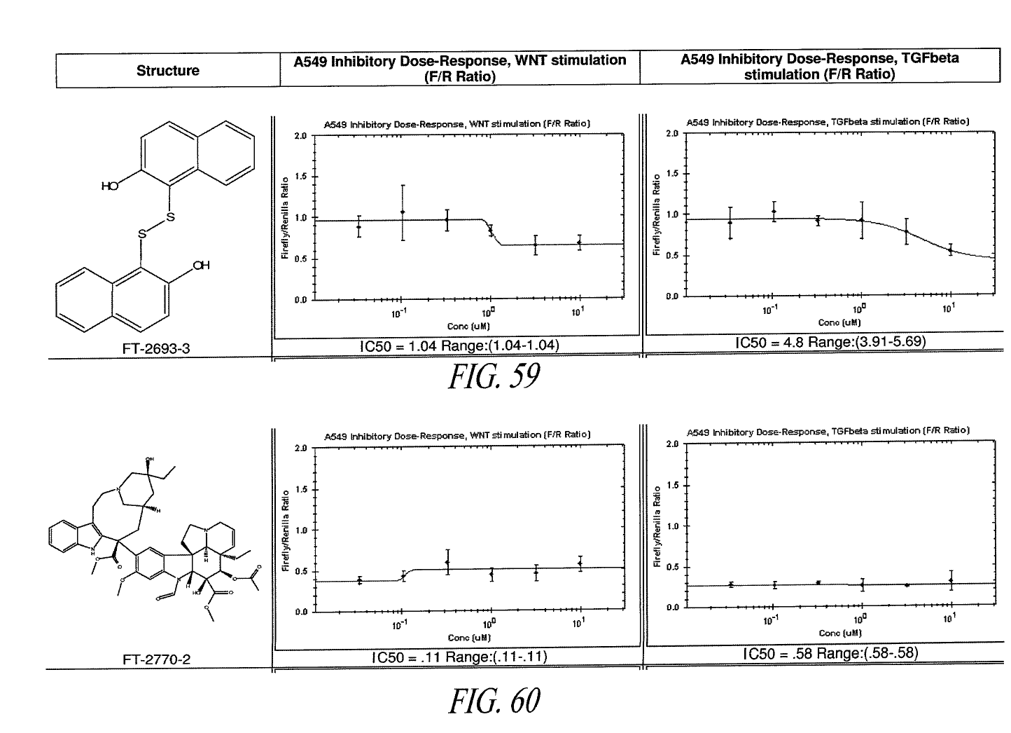

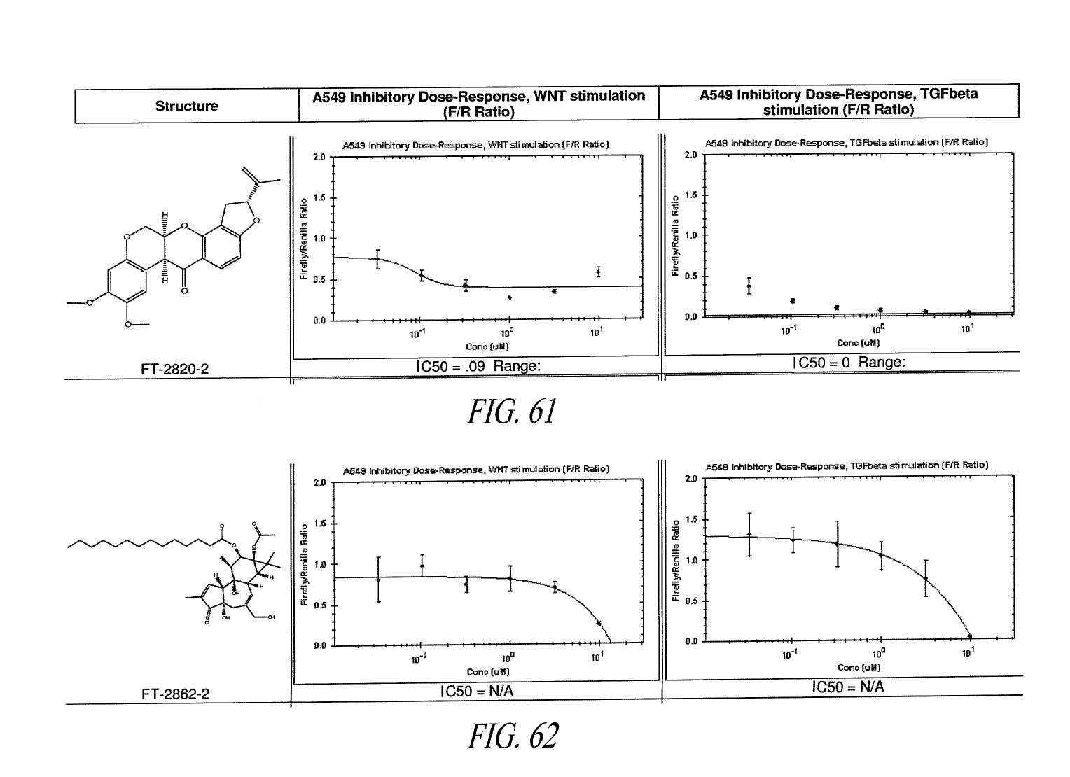

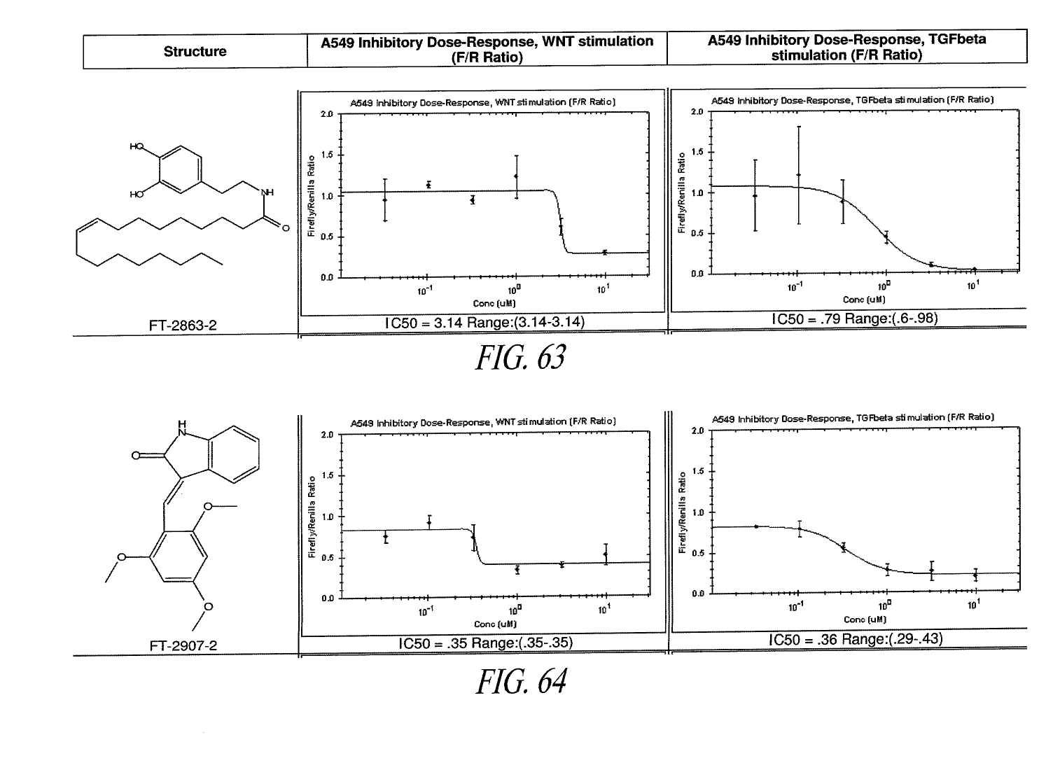

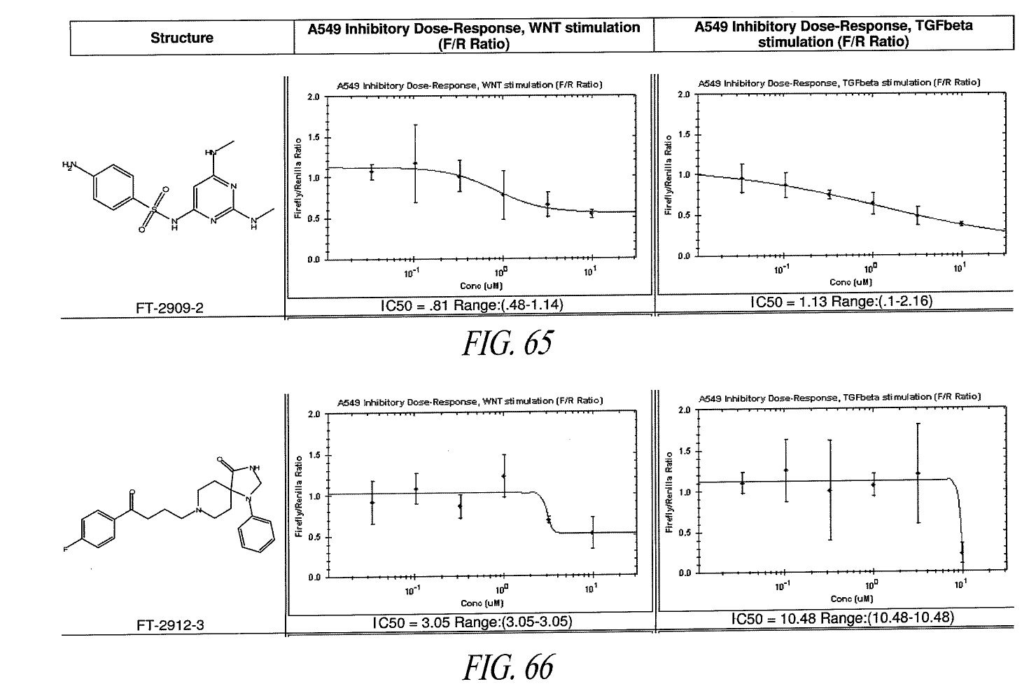

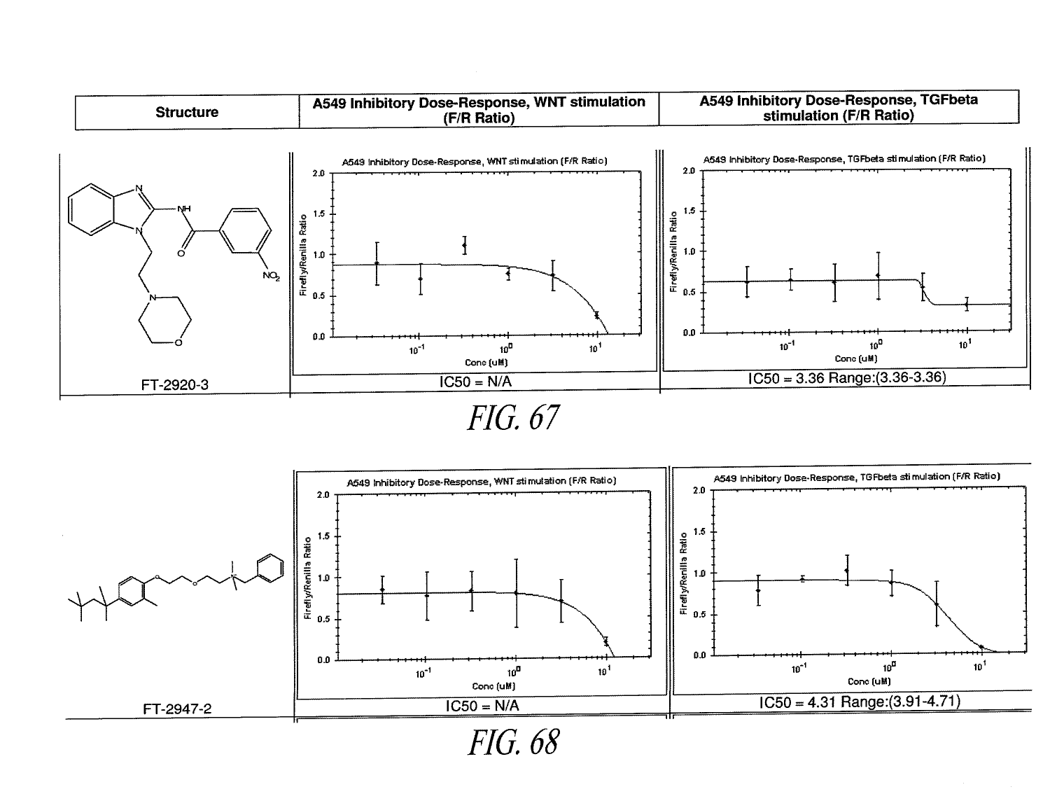

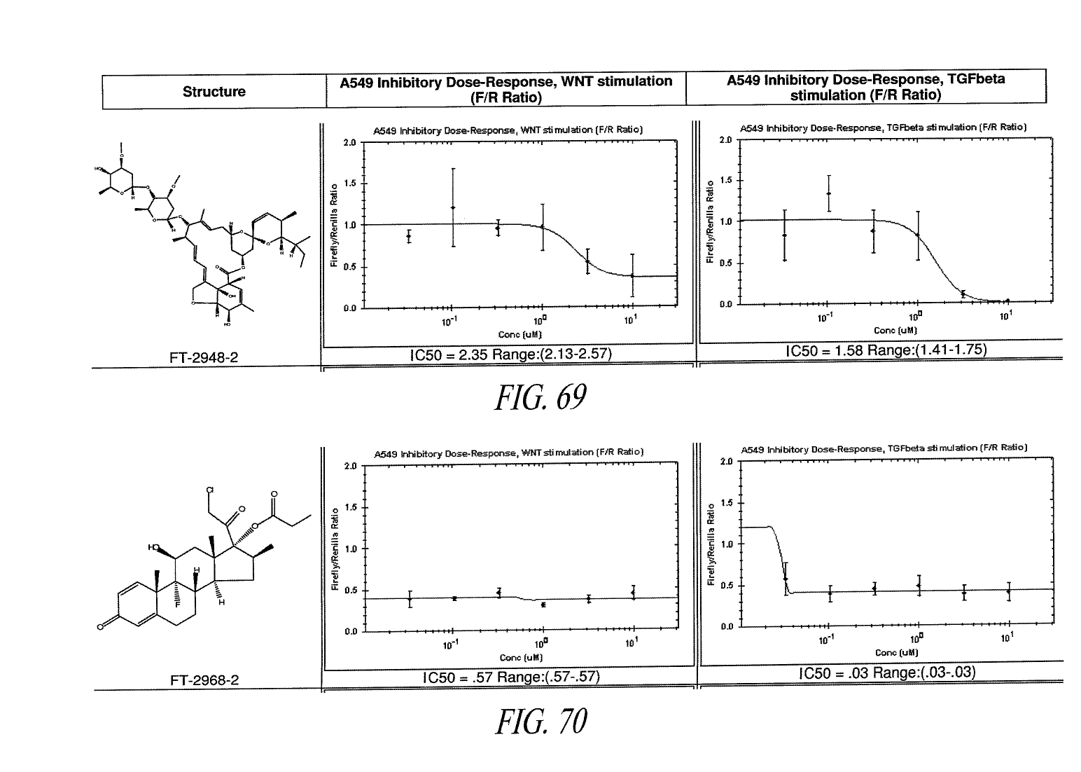

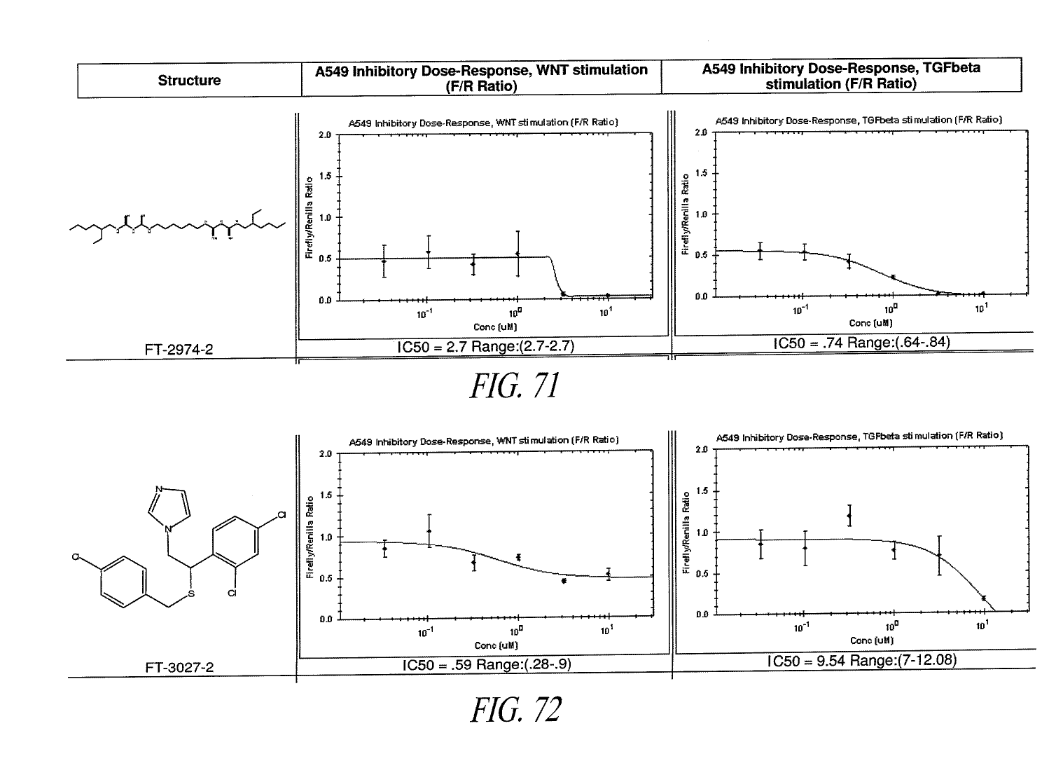

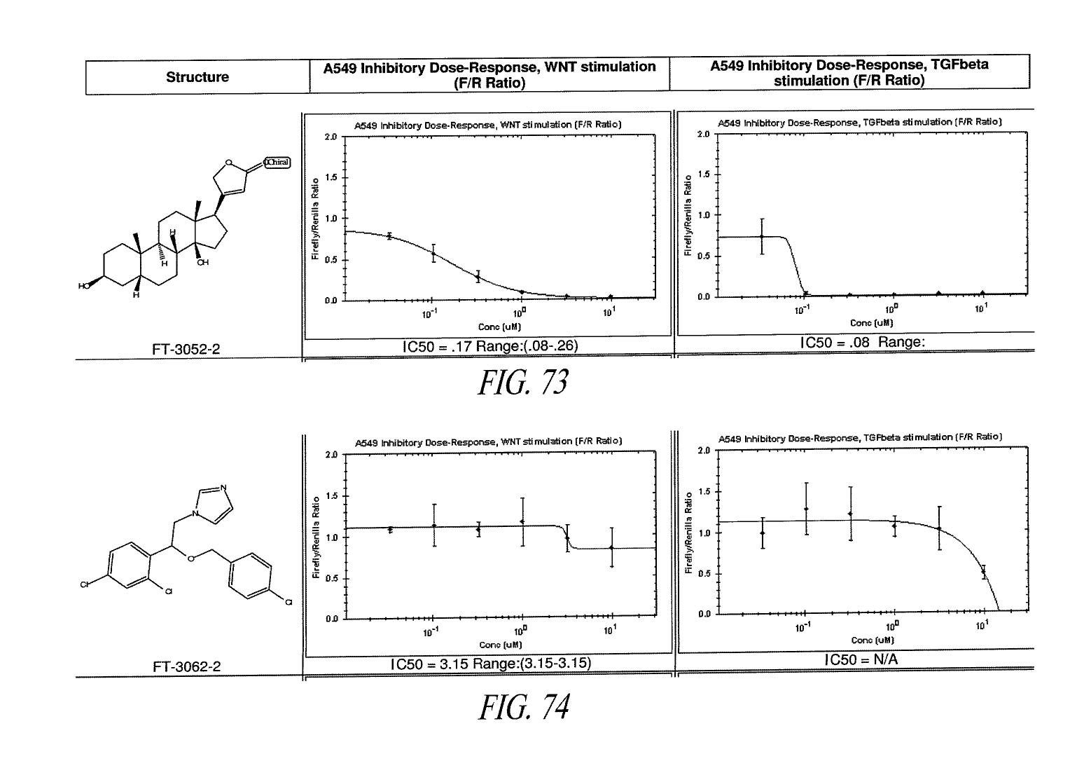

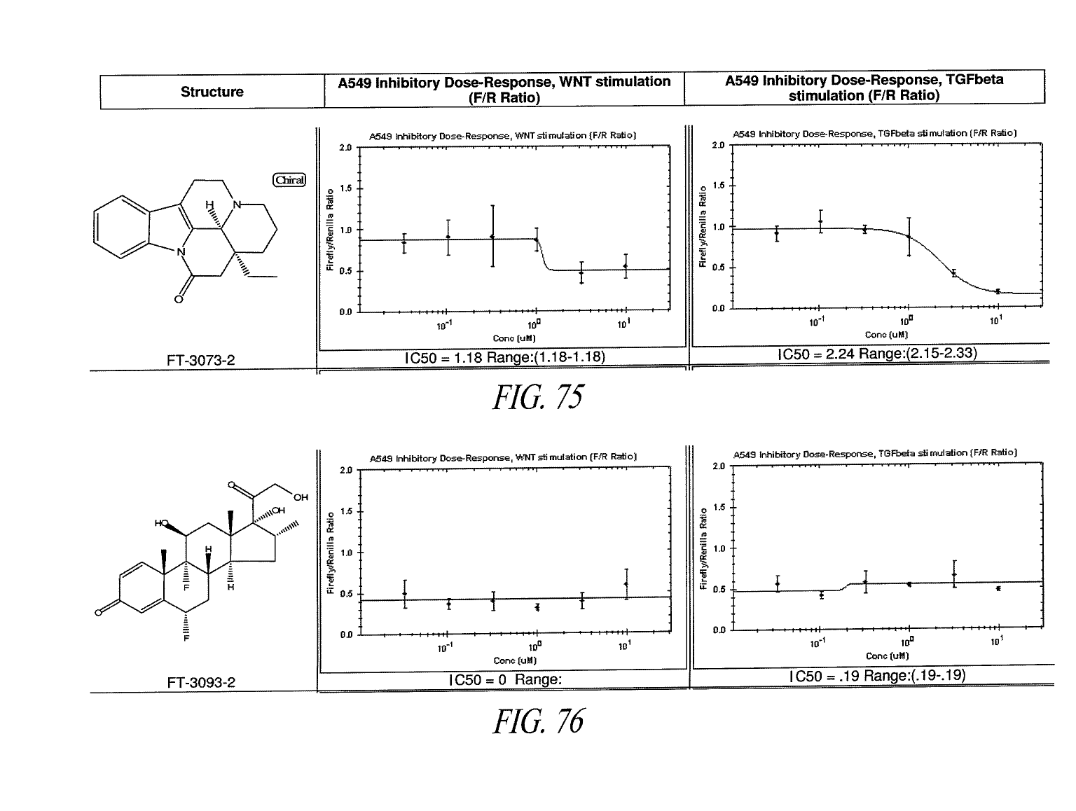

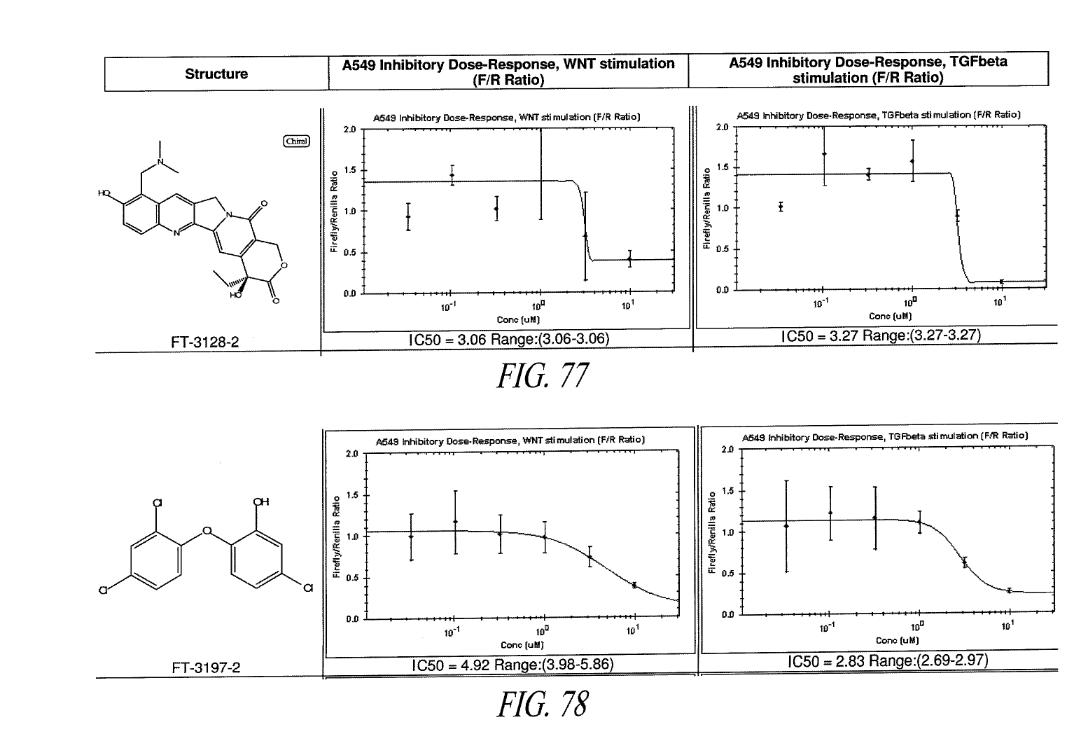

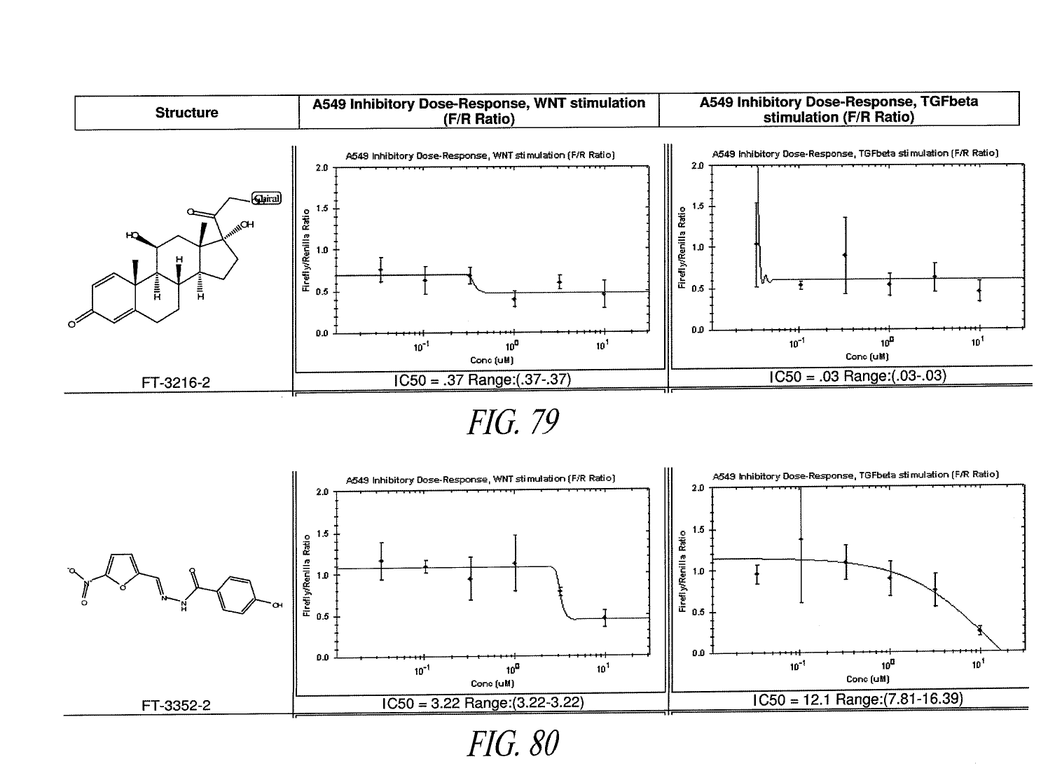

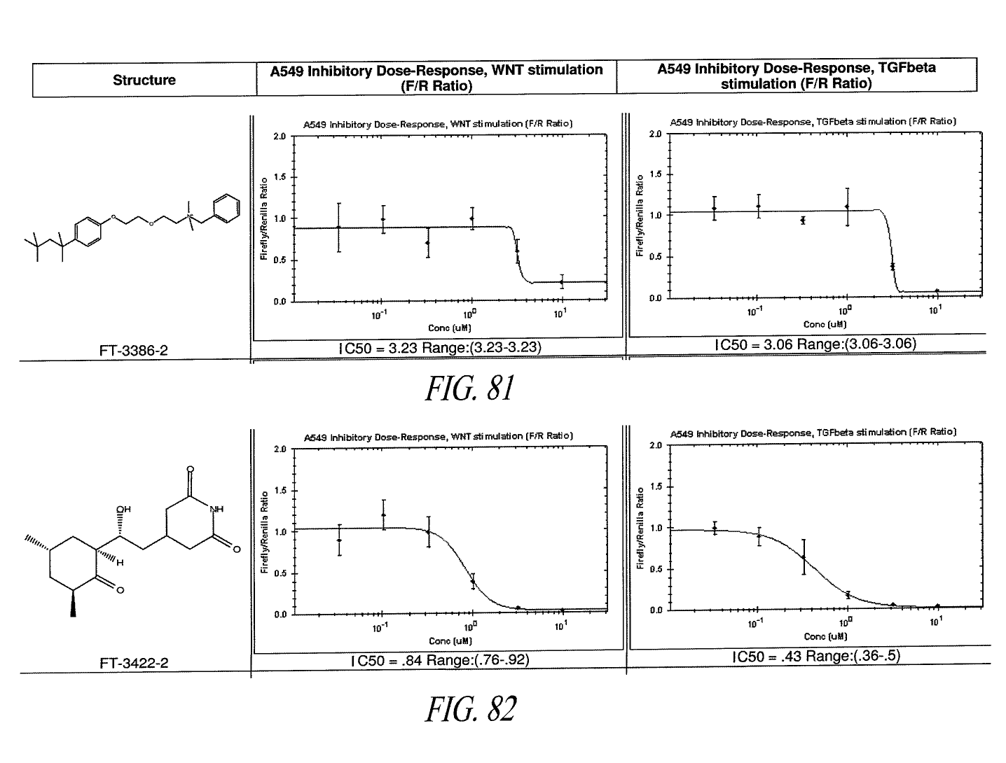

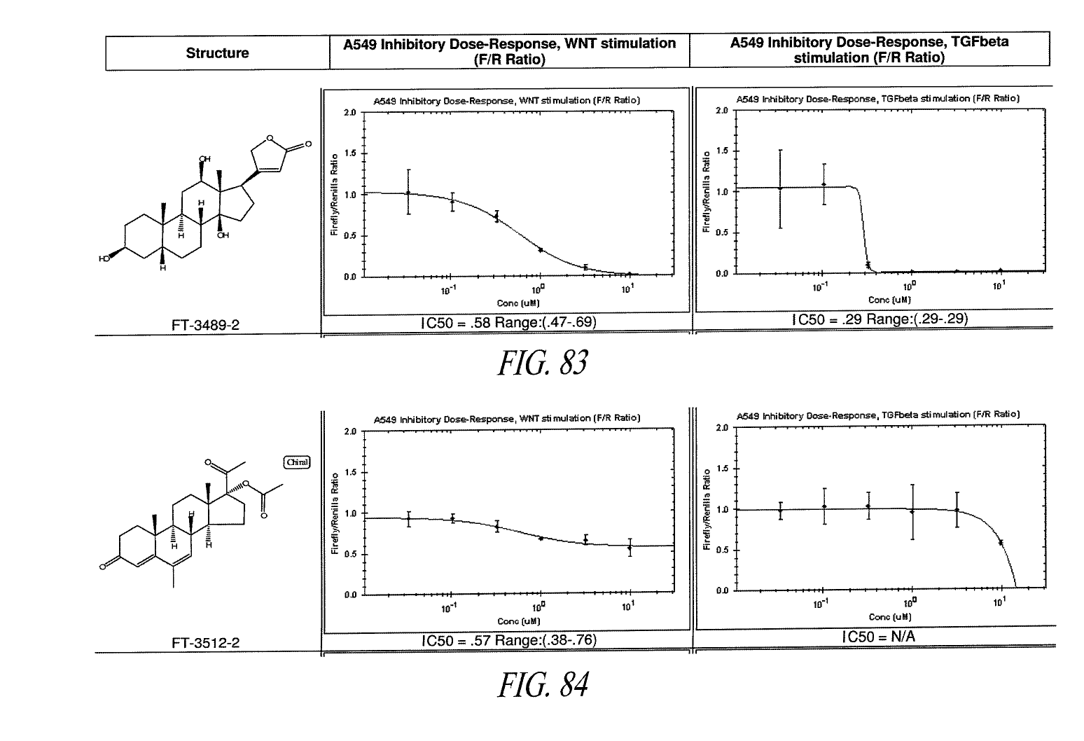

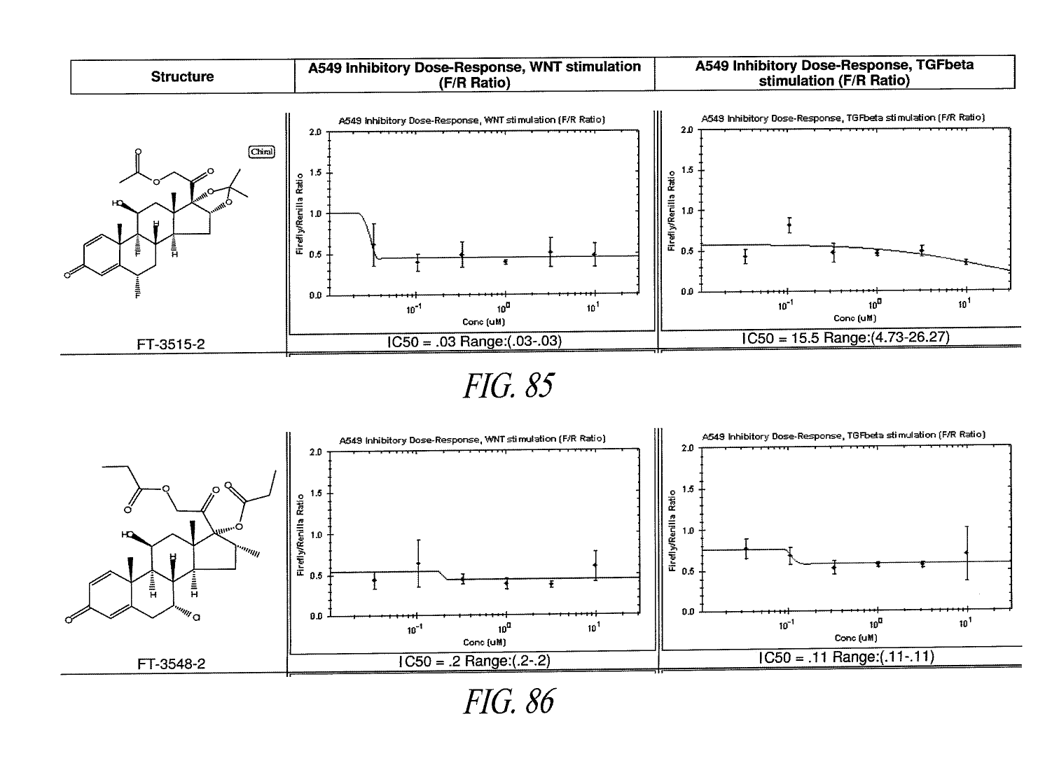

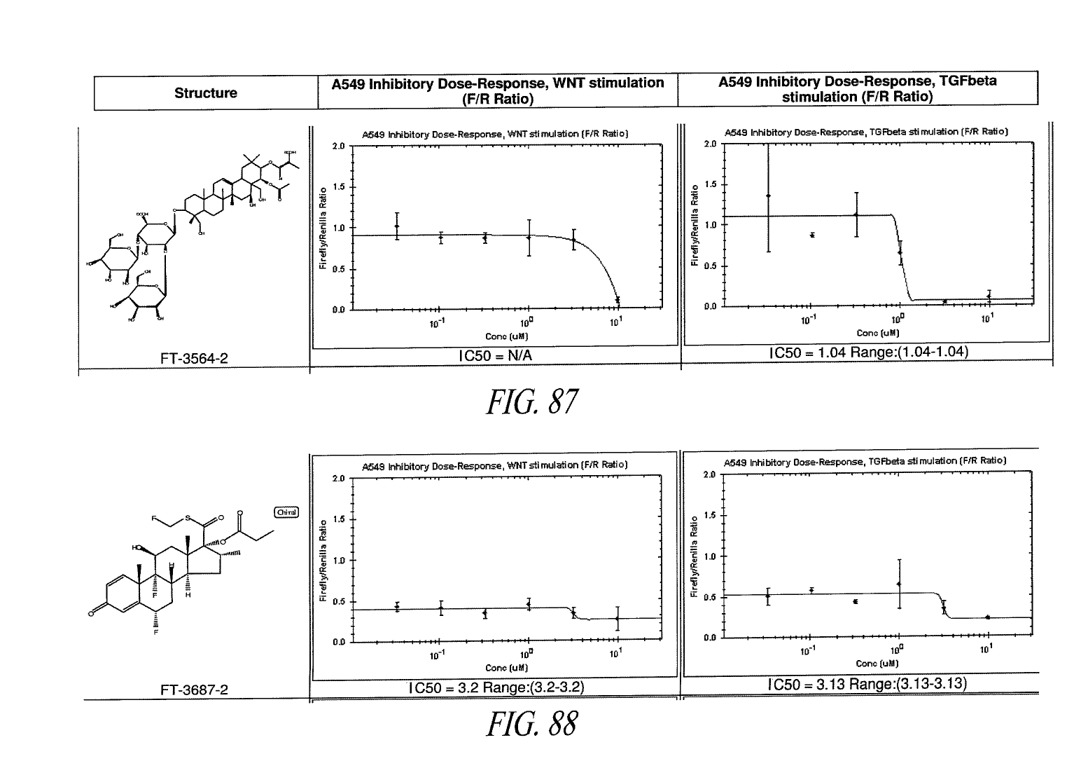

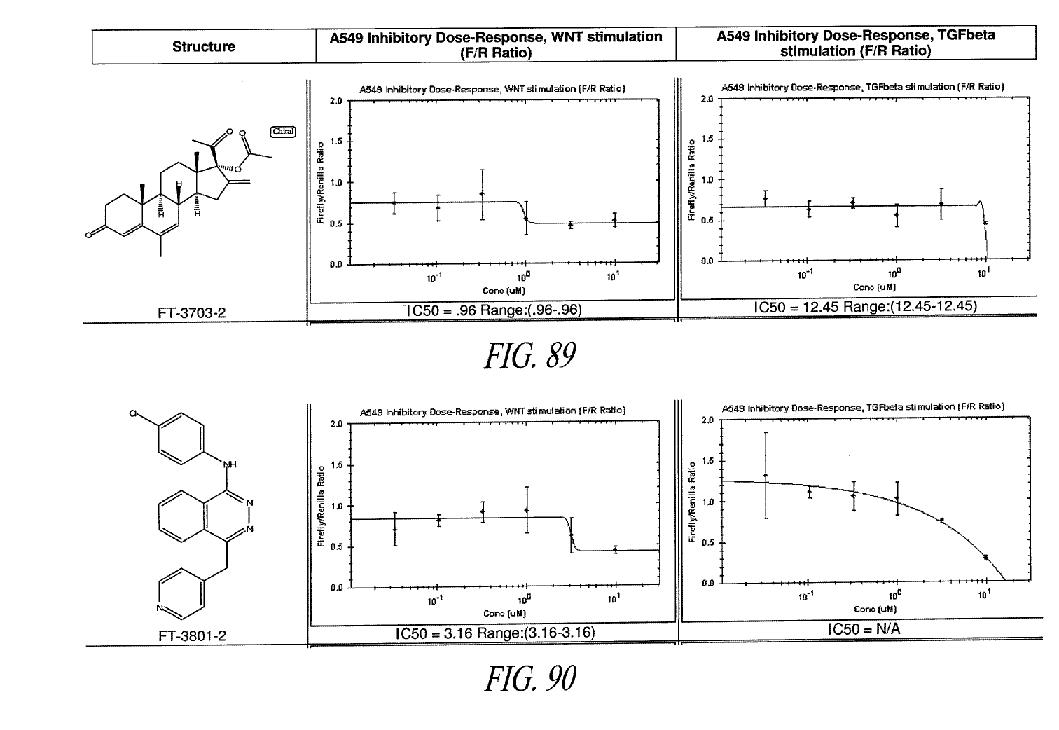

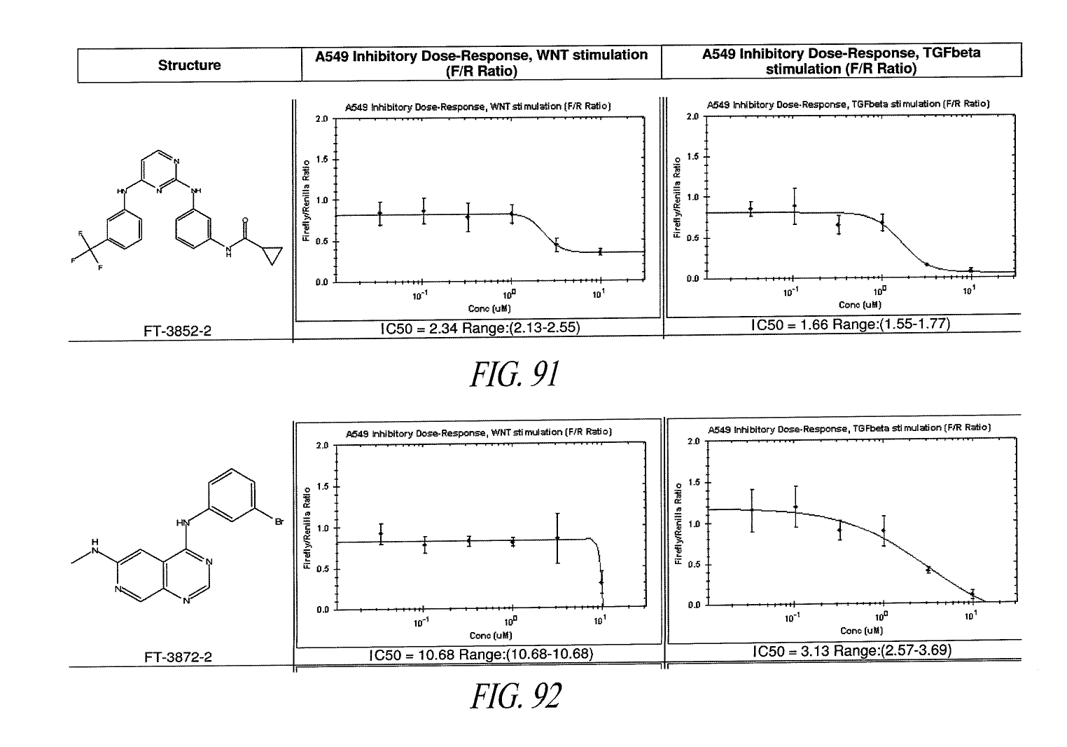

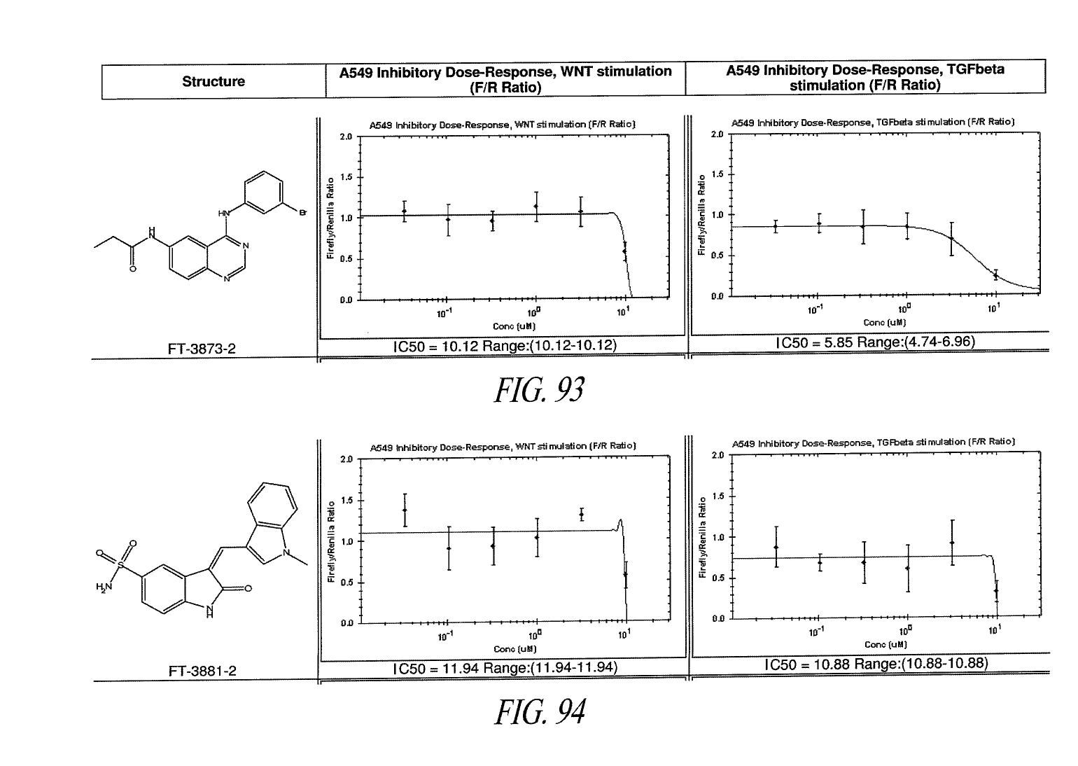

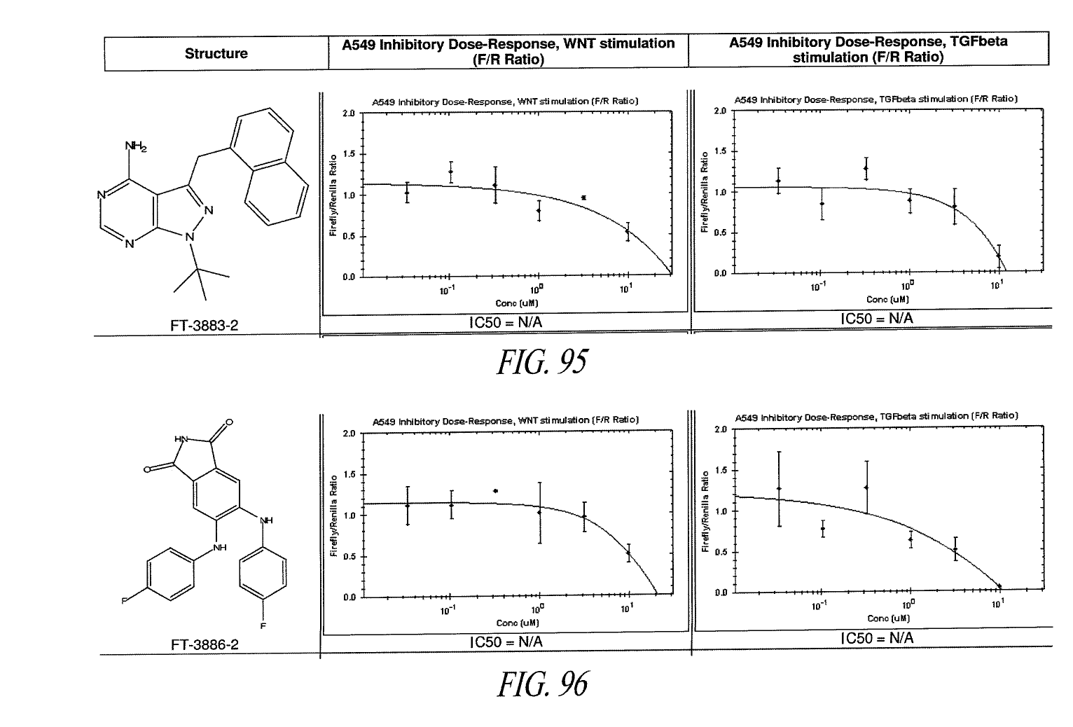

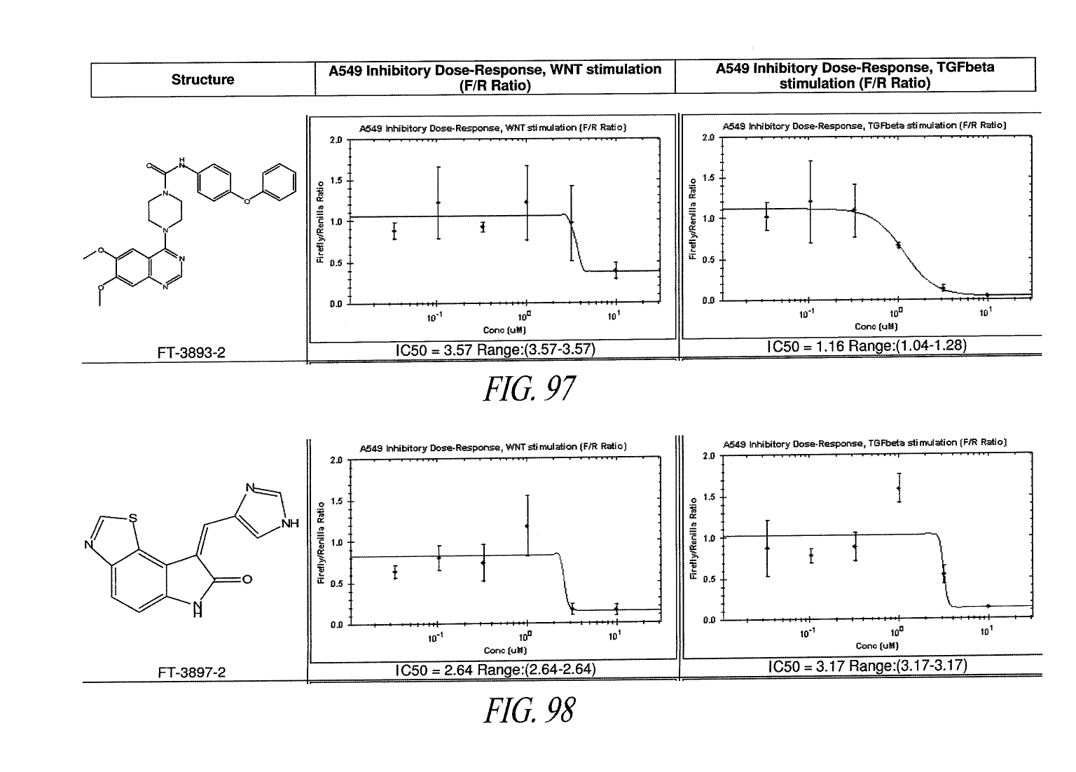

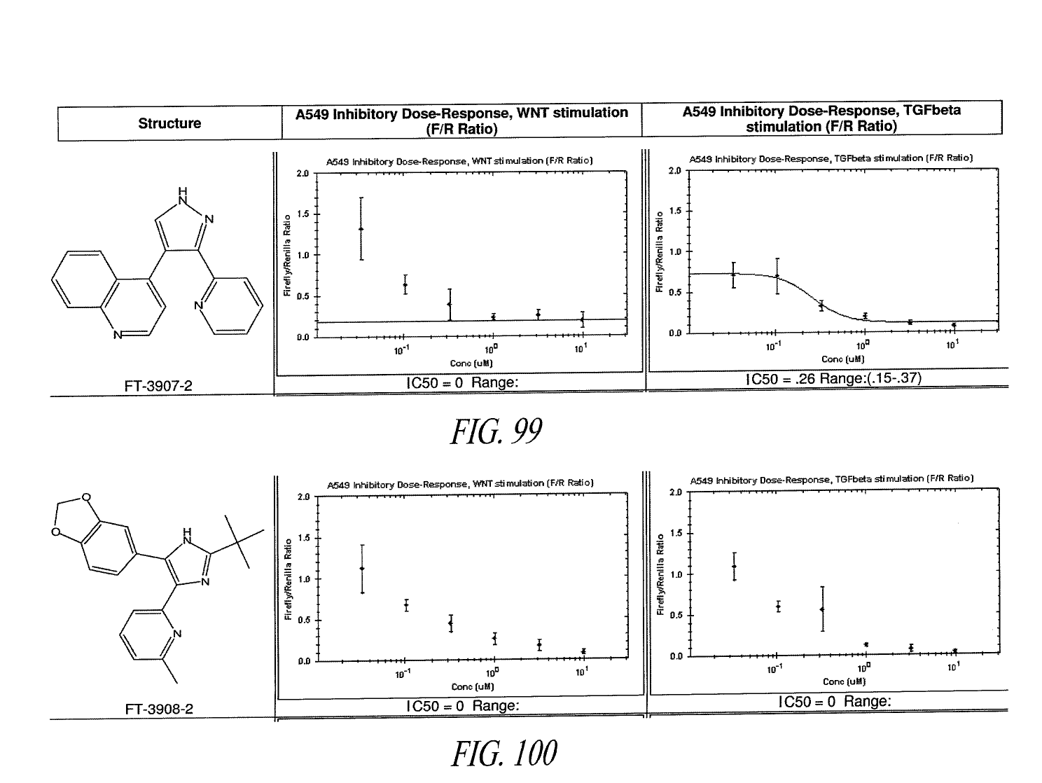

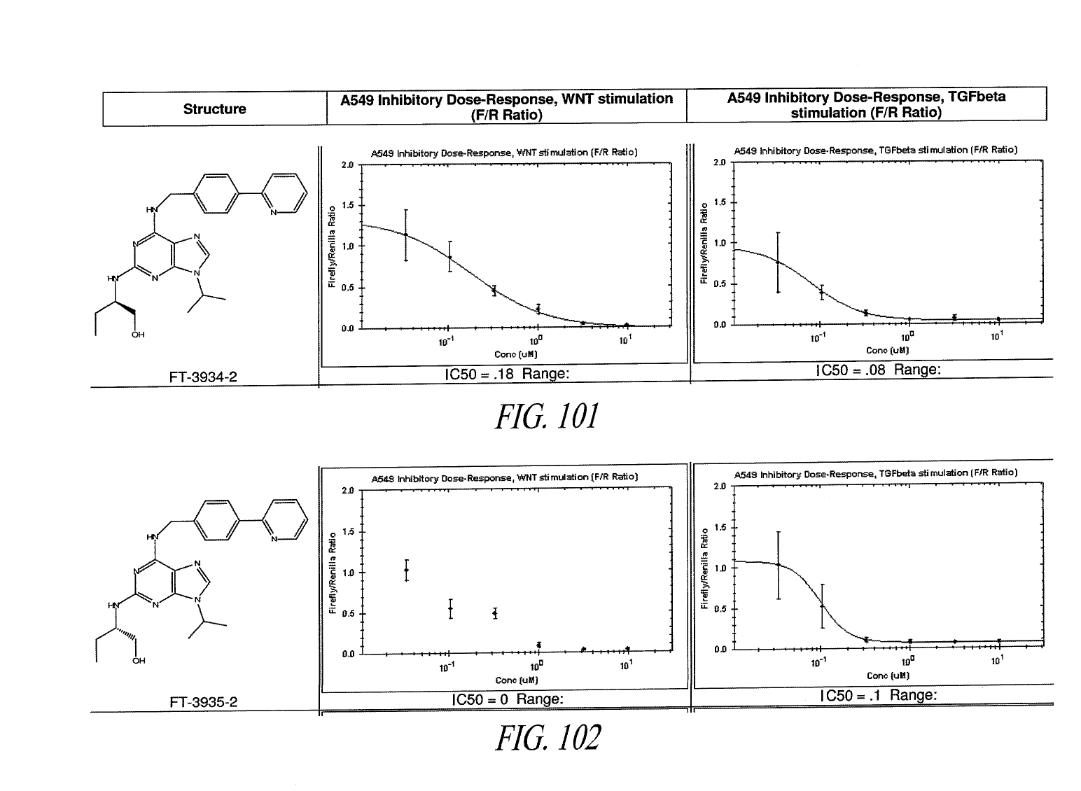

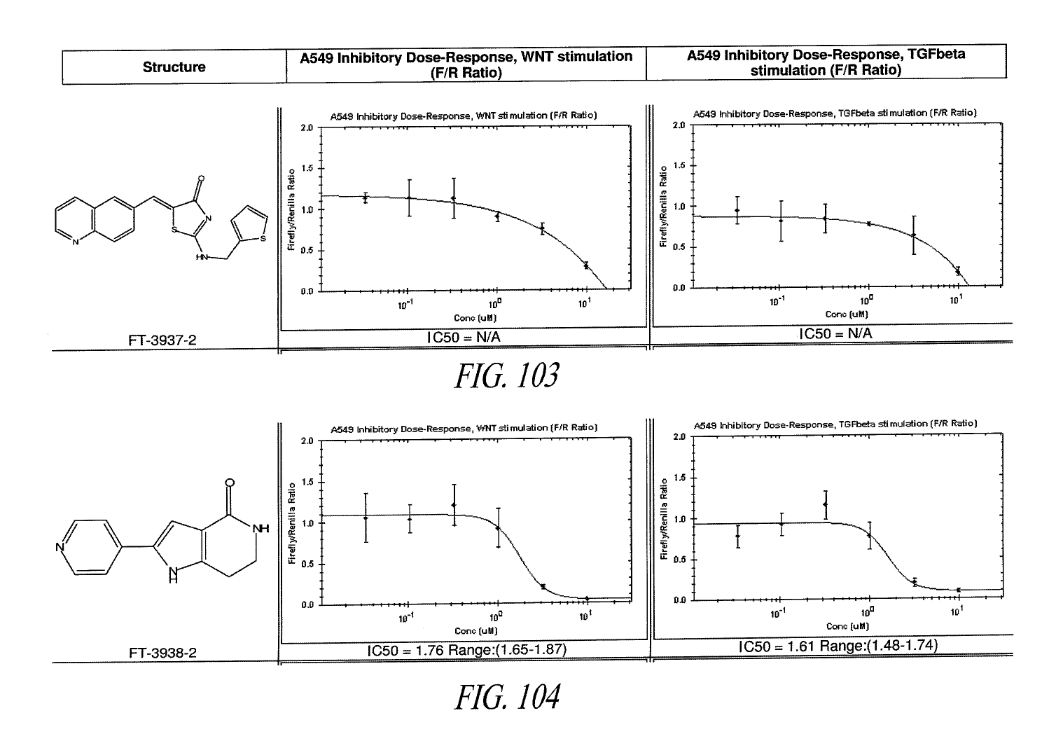

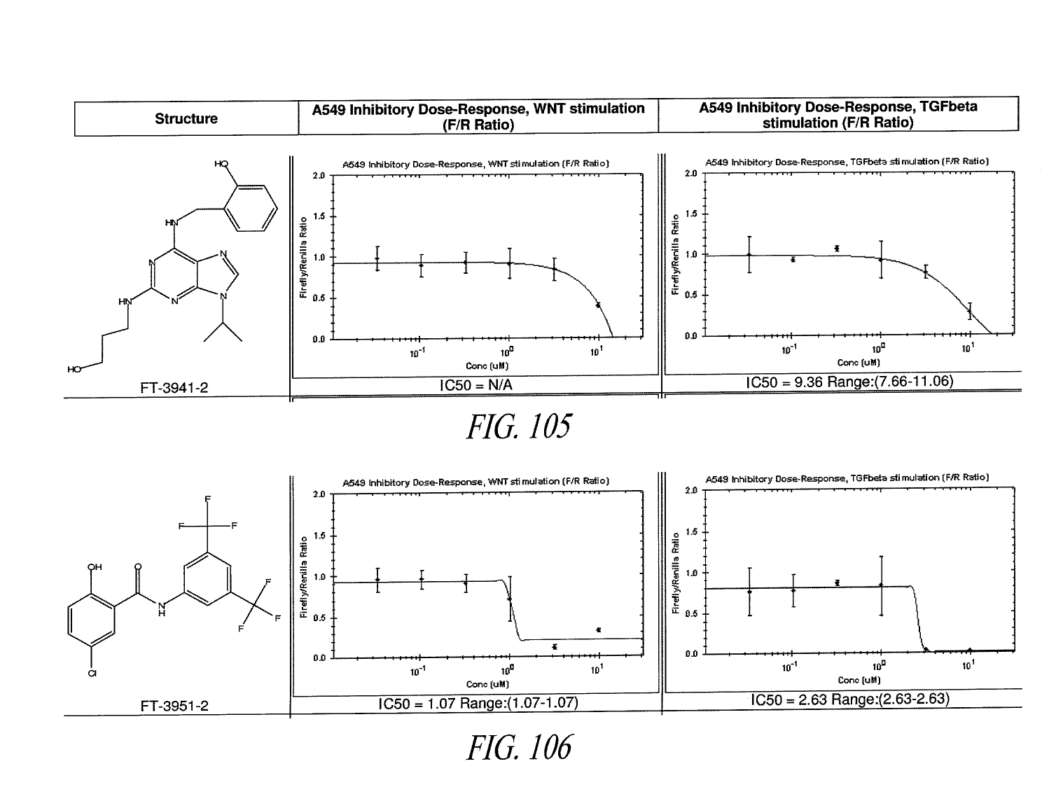

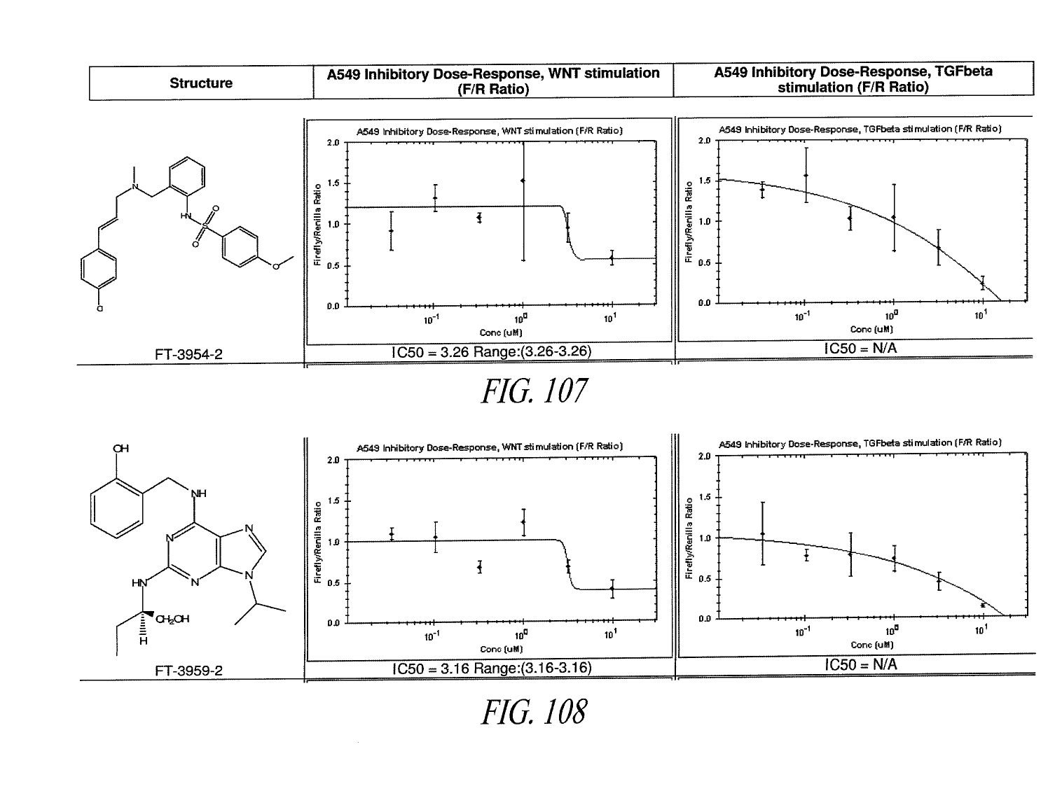

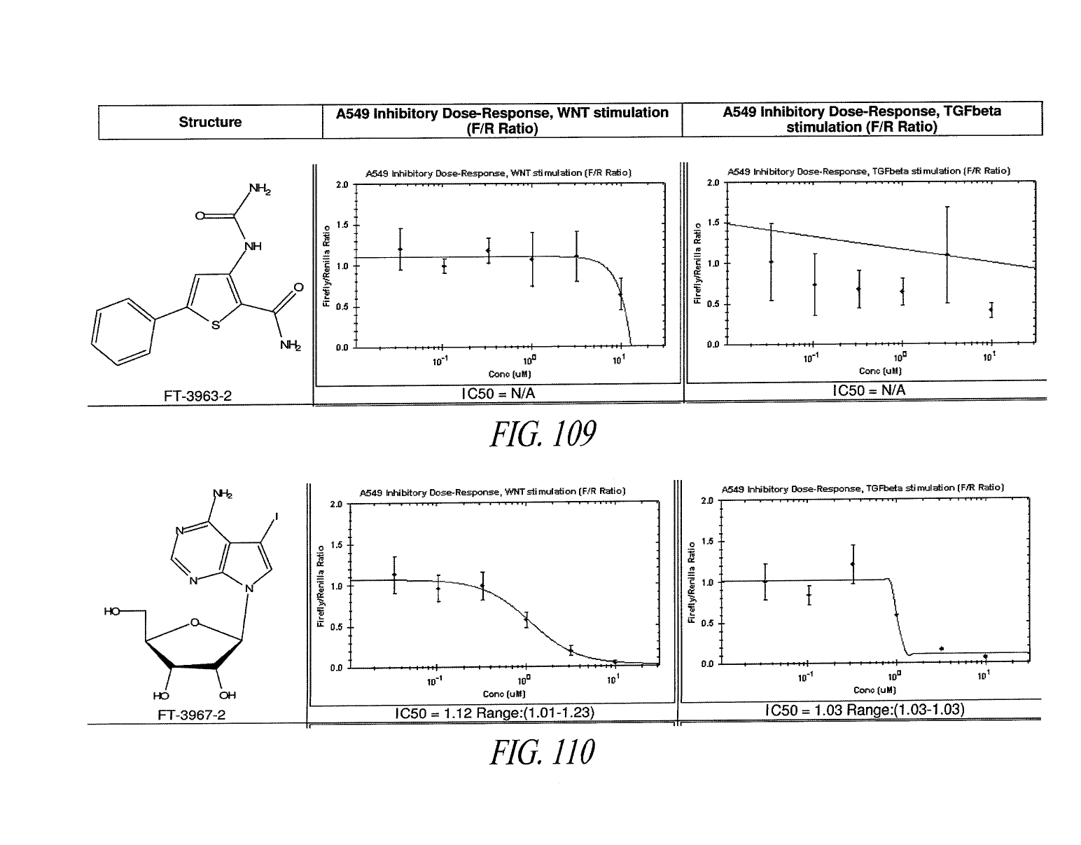

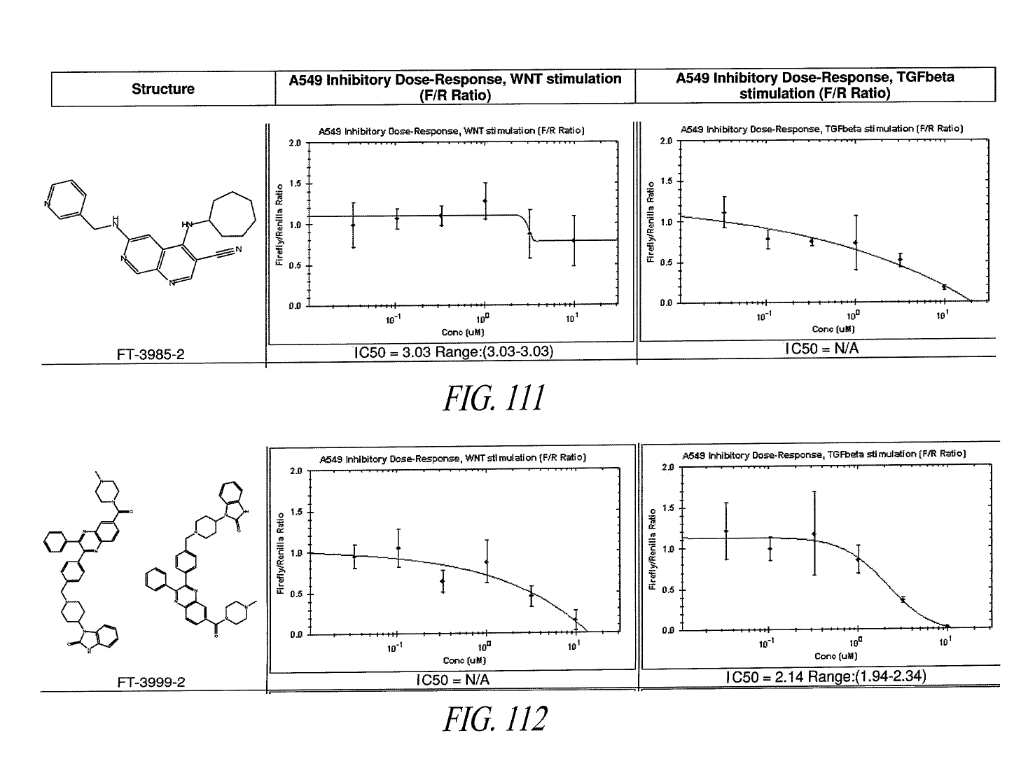

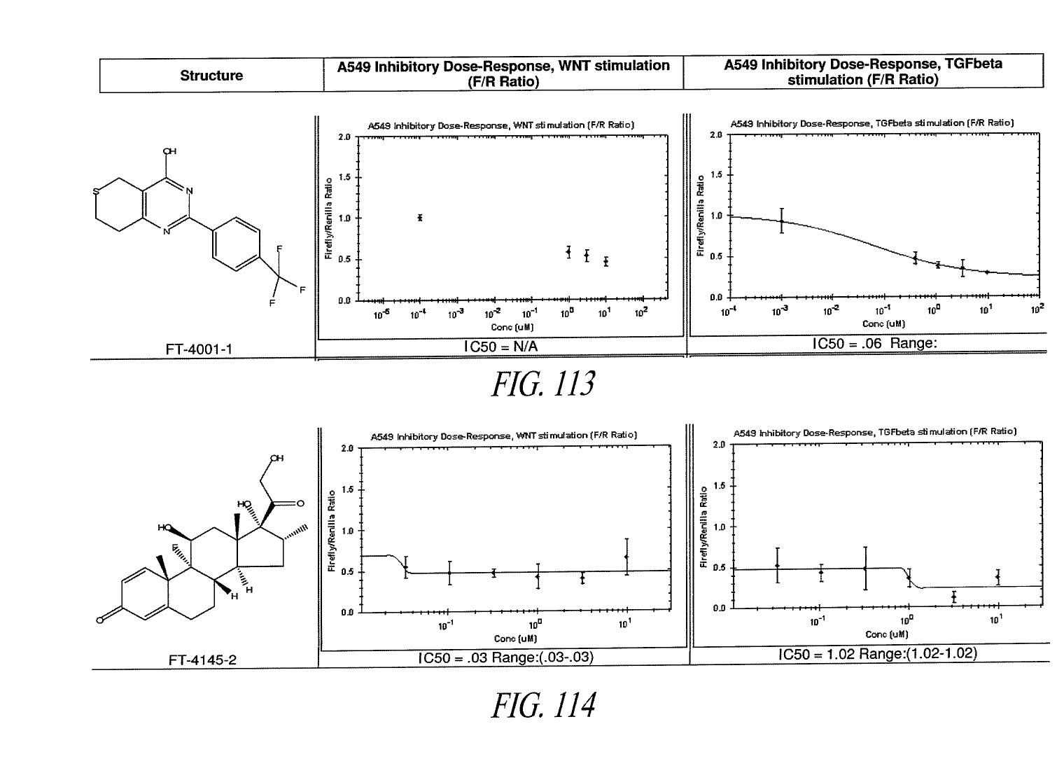

TABLE 1
small molecule inhibitors of Wnt- and TGF-β-mediated β-catenin signaling

| FIG. | Compound | IC50 in Wnt Stimulation | IC50 in TGF-β |
|---|---|---|---|
| number | Name | Assay | Stimulation Assay |
| FIG. 1 | FT-1055-3 | 3 Range: (2.86-3.14) | 1.28 Range: (.93-1.63) |
| FIG. 2 | FT-1067-3 | 1.36 Range: (1.28-1.44) | .53 Range: (.44-.62) |
| FIG. 3 | FT-1069-1 | N/A | N/A |
| FIG. 4 | FT-1083-1 | 3.26 Range: (3.26-3.26) | 1.5 Range: (1.35-1.65) |
| FIG. 5 | FT-1147-3 | 2.61 Range: (2.44-2.78) | N/A |
| FIG. 6 | FT-1150-3 | 10.88 Range: (10.88-10.88) | N/A |
| FIG. 7 | FT-1202-1 | 3.28 Range: (3.28-3.28) | 1.07 Range: (1.07-1.07) |
| FIG. 8 | FT-1203-1 | 10.76 Range: (7.58-13.94) | 1.48 Range: (.96-2) |
| FIG. 9 | FT-1812-4 | .87 Range: (.66-1.08) | .32 Range: (.32-.32) |
| FIG. 10 | FT-1265-1 | 1.92 Range: (1.33-2.51) | 1.4 Range: (.38-2.42) |
| FIG. 11 | FT-1281-1 | 1.03 Range: (1.03-1.03) | 2.96 Range: (2.96-2.96) |
| FIG. 12 | FT-1294-5 | 3.08 Range: (3.08-3.08) | 11.85 Range: (11.85-11.85) |
| FIG. 13 | FT-1301-1 | 5.23 Range: (3.66-6.8) | 1.45 Range: (1.36-1.54) |
| FIG. 14 | FT-1320-1 | .1 Range: (.1-.1) | 0 Range: |
| FIG. 15 | FT-1355-2 | N/A | 1.06 Range: (.21-1.91) |
| FIG. 16 | FT-1361-2 | N/A | 1.85 Range: (1.14-2.56) |
| FIG. 17 | FT-1366-2 | 2.46 Range: (2.32-2.6) | 1.29 Range: (1.16-1.42) |
| FIG. 18 | FT-1398-2 | 0 Range: | N/A |
| FIG. 19 | FT-1434-2 | 1.75 Range: (1.61-1.89) | 0 Range: |
| FIG. 20 | FT-1435-2 | .91 Range: (.03-1.79) | .7 Range: (.53-.87) |
| FIG. 21 | FT-1436-1 | N/A | 1.08 Range: (1.08-1.08) |
| FIG. 22 | FT-1480-1 | 3.19 Range: (3.19-3.19) | 1.28 Range: (1.07-1.49) |
| FIG. 23 | FT-1497-1 | .08 Range: | .09 Range: |
| FIG. 24 | FT-1504-3 | 2.68 Range: (2.23-3.13) | N/A |
| FIG. 25 | FT-1515-1 | 2.81 Range: (2.81-2.81) | 11.08 Range: (11.08-11.08) |
| FIG. 26 | FT-1517-1 | 3.01 Range: (3.01-3.01) | N/A |
| FIG. 27 | FT-1518-1 | 0 Range: | 3.15 Range: (3.15-3.15) |
| FIG. 28 | FT-1532-1 | 0 Range: | N/A |
| FIG. 29 | FT-1575-2 | .02 Range: (.02-.02) | N/A |
| FIG. 30 | FT-1609-1 | N/A | N/A |
| FIG. 31 | FT-1612-3 | .94 Range: (.94-.94) | .19 Range: (.19-.19) |
| FIG. 32 | FT-1613-1 | 3.16 Range: (3.16-3.16) | N/A |
| FIG. 33 | FT-1660-1 | .03 Range: (.03-.03) | .09 Range: (.09-.09) |
| FIG. 34 | FT-1678-1 | 2.07 Range: (2-2.14) | 4.07 Range: (2.99-5.15) |
| FIG. 35 | FT-1688-1 | 3.05 Range: (3.05-3.05) | 7.02 Range: |
| FIG. 36 | FT-1693-1 | .89 Range: (.89-.89) | .18 Range: (.18-.18) |
| FIG. 37 | FT-1812-3 | .75 Range: (.66-.84) | .25 Range: (.2-.3) |
| FIG. 38 | FT-1915-2 | .01 Range: (.01-.01) | .04 Range: |
| FIG. 39 | FT-1986-3 | N/A | 3.54 Range: (3.54-3.54) |
| FIG. 40 | FT-1992-3 | .32 Range: (.29-.35) | .16 Range: (.09-.23) |
| FIG. 41 | FT-2014-2 | 3.02 Range: (2.5-3.54) | 1.48 Range: (1.3-1.66) |
| FIG. 42 | FT-2046-2 | .24 Range: (.17-.31) | .15 Range: (.08-.22) |
| FIG. 43 | FT-2051-2 | 1 Range: (1-1) | 1.74 Range: (1.58-1.9) |
| FIG. 44 | FT-2081-2 | .09 Range: | .18 Range: |
| FIG. 45 | FT-2103-2 | 3.21 Range: (3.21-3.21) | .31 Range: (.31-.31) |
| FIG. 46 | FT-2115-2 | 1.06 Range: (.93-1.19) | .62 Range: (.41-.83) |
| FIG. 47 | FT-2228-3 | 2.74 Range: (2.26-3.22) | N/A |
| FIG. 48 | FT-2254-2 | .12 Range: | .11 Range: |
| FIG. 49 | FT-2318-2 | N/A | N/A |
| FIG. 50 | FT-2342-2 | 10.87 Range: (10.87-10.87) | 3.25 Range: (3.25-3.25) |
| FIG. 51 | FT-2474-2 | 2.24 Range: (1.93-2.55) | 6.88 Range: (5.93-7.83) |
| FIG. 52 | FT-2498-2 | 3.3 Range: (3.3-3.3) | 10.75 Range: |
| FIG. 53 | FT-2562-3 | 10.94 Range: (10.94-10.94) | 8.95 Range: (7.16-10.74) |
| FIG. 54 | FT-2580-2 | N/A | N/A |
| FIG. 55 | FT-2619-2 | .14 Range: (.05-.23) | .99 Range: (.99-.99) |
| FIG. 56 | FT-2633-2 | 0 Range: | .14 Range: (.05-.23) |
| FIG. 57 | FT-2660-2 | 3.81 Range: (3.81-3.81) | N/A |
| FIG. 58 | FT-2691-2 | .6 Range: (.6-.6) | .84 Range: (.84-.84) |
| FIG. 59 | FT-2693-3 | 1.04 Range: (1.04-1.04) | 4.8 Range: (3.91-5.69) |
| FIG. 60 | FT-2770-2 | .11 Range: (.11-.11) | .58 Range: (.58-.58) |
| FIG. 61 | FT-2820-2 | .09 Range: | 0 Range: |
| FIG. 62 | FT-2862-2 | N/A | N/A |
| FIG. 63 | FT-2863-2 | 3.14 Range: (3.14-3.14) | .79 Range: (.6-.98) |
| FIG. 64 | FT-2907-2 | .35 Range: (.35-.35) | .36 Range: (.29-.43) |
| FIG. 65 | FT-2909-2 | .81 Range: (.48-1.14) | 1.13 Range: (.1-2.16) |
| FIG. 66 | FT-2912-3 | 3.05 Range: (3.05-3.05) | 10.48 Range: (10.48-10.48) |
| FIG. 67 | FT-2920-3 | N/A | 3.36 Range: (3.36-3.36) |
| FIG. 68 | FT-2947-2 | N/A | 4.31 Range: (3.91-4.71) |
| FIG. 69 | FT-2948-2 | 2.35 Range: (2.13-2.57) | 1.58 Range: (1.41-1.75) |
| FIG. 70 | FT-2968-2 | .57 Range: (.57-.57) | .03 Range: (.03-.03) |
| FIG. 71 | FT-2974-2 | 2.7 Range: (2.7-2.7) | .74 Range: (.64-.84) |
| FIG. 72 | FT-3027-2 | .59 Range: (.28-.9) | 9.54 Range: (7-12.08) |
| FIG. 73 | FT-3052-2 | .17 Range: (.08-.26) | .08 Range: |
| FIG. 74 | FT-3062-2 | 3.15 Range: (3.15-3.15) | N/A |
| FIG. 75 | FT-3073-2 | 1.18 Range: (1.18-1.18) | 2.24 Range: (2.15-2.33) |
| FIG. 76 | FT-3093-2 | 0 Range: | .19 Range: (.19-.19) |
| FIG. 77 | FT-3128-2 | 3.06 Range: (3.06-3.06) | 3.27 Range: (3.27-3.27) |
| FIG. 78 | FT-3197-2 | 4.92 Range: (3.98-5.86) | 2.83 Range: (2.69-2.97) |
| FIG. 79 | FT-3216-2 | .37 Range: (.37-.37) | .03 Range: (.03-.03) |
| FIG. 80 | FT-3352-2 | 3.22 Range: (3.22-3.22) | 12.1 Range: (7.81-16.39) |
| FIG. 81 | FT-3386-2 | 3.23 Range: (3.23-3.23) | 3.06 Range: (3.06-3.06) |
| FIG. 82 | FT-3422-2 | .84 Range: (.76-.92) | .43 Range: (.36-.5) |
| FIG. 83 | FT-3489-2 | .58 Range: (.47-.69) | .29 Range: (.29-.29) |
| FIG. 84 | FT-3512-2 | .57 Range: (.38-.76) | N/A |
| FIG. 85 | FT-3515-2 | .03 Range: (.03-.03) | 15.5 Range: (4.73-26.27) |
| FIG. 86 | FT-3548-2 | .2 Range: (.2-.2) | .11 Range: (.11-.11) |
| FIG. 87 | FT-3564-2 | N/A | 1.04 Range: (1.04-1.04) |
| FIG. 88 | FT-3687-2 | 3.2 Range: (3.2-3.2) | 3.13 Range: (3.13-3.13) |
| FIG. 89 | FT-3703-2 | .96 Range: (.96-.96) | 12.45 Range: (12.45-12.45) |
| FIG. 90 | FT-3801-2 | 3.16 Range: (3.16-3.16) | N/A |
| FIG. 91 | FT-3852-2 | 2.34 Range: (2.13-2.55) | 1.66 Range: (1.55-1.77) |
| FIG. 92 | FT-3872-2 | 10.68 Range: (10.68-10.68) | 3.13 Range: (2.57-3.69) |
| FIG. 93 | FT-3873-2 | 10.12 Range: (10.12-10.12) | 5.85 Range: (4.74-6.96) |
| FIG. 94 | FT-3881-2 | 11.94 Range: (11.94-11.94) | 10.88 Range: (10.88-10.88) |
| FIG. 95 | FT-3883-2 | N/A | N/A |
| FIG. 96 | FT-3886-2 | N/A | N/A |
| FIG. 97 | FT-3893-2 | 3.57 Range: (3.57-3.57) | 1.16 Range: (1.04-1.28) |
| FIG. 98 | FT-3897-2 | 2.64 Range: (2.64-2.64) | 3.17 Range: (3.17-3.17) |
| FIG. 99 | FT-3907-2 | 0 Range: | .26 Range: (.15-.37) |
| FIG. 100 | FT-3908-2 | 0 Range: | 0 Range: |
| FIG. 101 | FT-3934-2 | .18 Range: | .08 Range: |
| FIG. 102 | FT-3935-2 | 0 Range: | .1 Range: |
| FIG. 103 | FT-3937-2 | N/A | N/A |
| FIG. 104 | FT-3938-2 | 1.76 Range: (1.65-1.87) | 1.61 Range: (1.48-1.74) |
| FIG. 105 | FT-3941-2 | N/A | 9.36 Range: (7.66-11.06) |
| FIG. 106 | FT-3951-2 | 1.07 Range: (1.07-1.07) | 2.63 Range: (2.63-2.63) |
| FIG. 107 | FT-3954-2 | 3.26 Range: (3.26-3.26) | N/A |
| FIG. 108 | FT-3959-2 | 3.16 Range: (3.16-3.16) | N/A |
| FIG. 109 | FT-3963-2 | N/A | N/A |
| FIG. 110 | FT-3967-2 | 1.12 Range: (1.01-1.23) | 1.03 Range: (1.03-1.03) |
| FIG. 111 | FT-3985-2 | 3.03 Range: (3.03-3.03) | N/A |
| FIG. 112 | FT-3999-2 | N/A | 2.14 Range: (1.94-2.34) |
| FIG. 113 | FT-4001-1 | N/A | .06 Range: |
| FIG. 114 | FT-4145-2 | .03 Range: (.03-.03) | 1.02 Range: (1.02-1.02) |

### Example 2

### Wnt- and TGF-b-Mediated b-Catenin Signaling Antagonists Inhibit TGF-b Mediated Emt in Mouse Type II Alveolar Cells

Mouse type II alveolar cells (ATII) were isolated from adult mice (6-12 weeks old) as described by Corti et al., Am J Respir Cell Mol Biol. 1996; 14, 309-315. ATII cells were cultured on fibronectin-coated tissue culture plates in SAGM medium (Lonza) containing 5% charcoal-treated fetal bovine serum+10 ng/mL KGF and either a 1:2000 dilution of DMSO (a negative control), 5 uM FT-2097, 5 uM FT-3934, 5 uM FT-4001 or 5 uM SB431542 (a positive control that inhibits TGF-β signaling). ATII cells cultured on fibronectin induced EMT by stimulating a TGF-β autocrine/paracrine loop. ATII cells were retreated after two days and cultured for an additional 3 days, which provided 5 days of total treatment.

Inhibition of EMT by treated with DMSO (a negative control), 5 uM FT-2097, 5 uM FT-3934, 5 uM FT-4001 and 5 uM SB431542 was determined by analyzing the amount of the EMT marker, smooth muscle actin (SMA) in the treated cells. ATII cells were lysed in RIPA buffer (150 mM NaCl, 50 mM Tris pH8.0, 1% Triton X-100, 0.5% sodium deoxycholate and 0.1% sodium dodecyl sulfate) supplemented with protease and phosphatase inhibitors. ATII cell lysates were processed by gel electrophoresis and western blotting with anti-SMA and GAPDH antibodies.

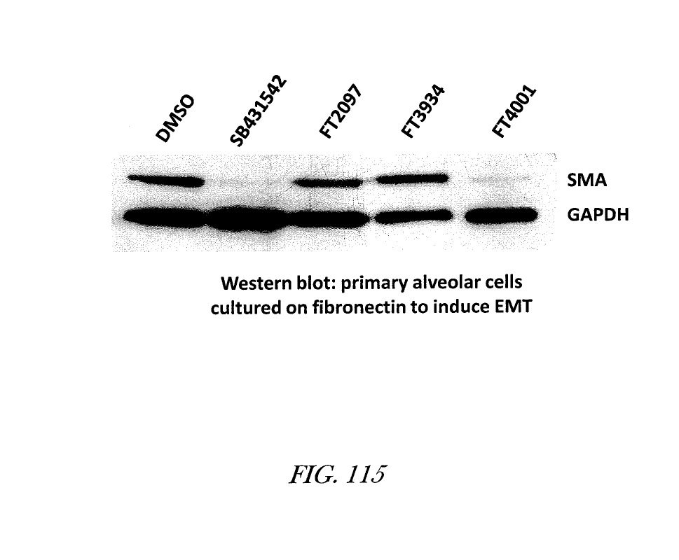

FIG. 115 shows the results from a representative experiment. The results indicate that FT4001 inhibited EMT in primary mouse alveolar cells.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of preventing or reducing fibrosis comprising inhibiting Wnt- and TGF-β-mediated β-catenin signaling.

2. The method of claim 1, comprising administering one or more inhibitors of β-catenin signaling, wherein the inhibitor inhibits Wnt- and TGF-β-mediated β-catenin signaling.

3. The method of claim 1, wherein the fibrosis is associated with a fibroproliferative disease selected from the group consisting of: kidney fibrosis, liver fibrosis, lung fibrosis, and systemic sclerosis.

4. The method of claim 3, wherein the fibroproliferative disease is idiopathic pulmonary fibrosis.

5. A method of preventing or treating lung fibrosis in a subject comprising administering one or more inhibitors of β-catenin signaling to the subject, wherein the inhibitor inhibits Wnt- and TGFβ-mediated β-catenin signaling.

6. The method of claim 5, wherein the lung fibrosis is idiopathic pulmonary fibrosis.

7. A method of inhibiting epithelial to mesenchymal transition (EMT) in an epithelial cell comprising contacting the epithelial cell with an inhibitor of β-catenin signaling, wherein the inhibitor inhibits Wnt- and TGF-β-mediated β-catenin signaling.

8. The method of claim 7, wherein the cell is a lung cell, a kidney cell, or a liver cell.

9. A method of inhibiting endothelial to mesenchymal transition (EnMT) in an endothelial cell comprising contacting the endothelial cell with an inhibitor of O-catenin signaling, wherein the inhibitor inhibits Wnt- and TGF-β-mediated β-catenin signaling.

10. The method of claim 9, wherein the cell is a lung cell, a kidney cell, or a liver cell.

11. A method of inhibiting myofibroblast activation in a myofibroblast comprising contacting the myofibroblast with an inhibitor of β-catenin signaling, wherein the inhibitor inhibits Wnt- and TGF-β-mediated β-catenin signaling.

12. The method of claim 11, wherein the myofibroblast is a present in a lung tissue, a kidney tissue, or a liver tissue.

13. A pharmaceutical composition comprising an inhibitor of O-catenin signaling, wherein the inhibitor inhibits Wnt- and TGF-β-mediated β-catenin signaling, and a pharmaceutically acceptable carrier or excipient, wherein the composition prevents or reduces fibrosis.

14. The composition of claim 13, wherein the fibrosis is associated with a fibroproliferative disease selected from the group consisting of: kidney fibrosis, liver fibrosis, lung fibrosis, and systemic sclerosis.

15. The composition of claim 14, wherein the fibroproliferative disease is idiopathic pulmonary fibrosis

16. (canceled)

17. The method of claim 1, wherein the inhibitor comprises a small molecule.

18. The method of claim 17, wherein the inhibitor is selected from the group consisting of: FT-1055-3, FT-1067-3, FT-1069-1, FT-1083-1, FT-1147-3, FT-1150-3, FT-1202-1, FT-1203-1, FT-1812-4, FT-1265-1, FT-1281-1, FT-1294-5, FT-1301-1, FT-1320-1, FT-1355-2, FT-1361-2, FT-1366-2, FT-1398-2, FT-1434-2, FT-1435-2, FT-1436-1, FT-1480-1, FT-1497-1, FT-1504-3, FT-1515-1, FT-1517-1, FT-1518-1, FT-1532-1, FT-1575-2, FT-1609-1, FT-1612-3, FT-1613-1, FT-1660-1, FT-1678-1, FT-1688-1, FT-1693-1, FT-1812-3, FT-1915-2, FT-1986-3, FT-1992-3, FT-2014-2, FT-2046-2, FT-2051-2, FT-2081-2, FT-2103-2, FT-2115-2, FT-2228-3, FT-2254-2, FT-2318-2, FT-2342-2, FT-2474-2, FT-2498-2, FT-2562-3, FT-2580-2, FT-2619-2, FT-2633-2, FT-2660-2, FT-2691-2, FT-2693-3, FT-2770-2, FT-2820-2, FT-2862-2, FT-2863-2, FT-2907-2, FT-2909-2, FT-2912-3, FT-2920-3, FT-2947-2, FT-2948-2, FT-2968-2, FT-2974-2, FT-3027-2, FT-3052-2, FT-3062-2, FT-3073-2, FT-3093-2, FT-3128-2, FT-3197-2, FT-3216-2, FT-3352-2, FT-3386-2, FT-3422-2, FT-3489-2, FT-3512-2, FT-3515-2, FT-3548-2, FT-3564-2, FT-3687-2, FT-3703-2, FT-3801-2, FT-3852-2, FT-3872-2, FT-3873-2, FT-3881-2, FT-3883-2, FT-3886-2, FT-3893-2, FT-3897-2, FT-3907-2, FT-3908-2, FT-3934-2, FT-3935-2, FT-3937-2, FT-3938-2, FT-3941-2, FT-3951-2, FT-3954-2, FT-3959-2, FT-3963-2, FT-3967-2, FT-3985-2, FT-3999-2, FT-4001-1, and FT-4145-2.

19. The method of claim 17, wherein the inhibitor is selected from the group consisting of: FT-1067, FT-2907, FT-3934, FT-3938, FT-3951, FT-3967, and FT-4001.
